(19) United States
(12) Patent Application Publication
(10) Pub. No.: US 2021/0315986 A1
(43) Pub. Date: Oct. 14, 2021

(54) PSMA AND STEAP1 VACCINES AND THEIR USES

(71) Applicant: Janssen Biotech, Inc., Horsham, PA (US)

(72) Inventors: Marco Gottardis, Princeton, NJ (US); Selina Khan, Leiden (NL); Manuel Alejandro Sepulveda, West Windsor, NJ (US); Douglas H. Yamada, Ardmore, PA (US); Roland Zahn, CN Leiden (NL); Brent Rupnow, Raritan, NJ (US)

(21) Appl. No.: 17/228,871

(22) Filed: Apr. 13, 2021

Related U.S. Application Data
(60) Provisional application No. 63/008,848, filed on Apr. 13, 2020.
(60) Provisional application No. 63/158,601, filed on Mar. 9, 2021.

(51) Int. Cl. A61K 39/00; A61K 45/06; C12N 15/86; A61P 35/00

(52) U.S. Cl. CPC A61K 39/001195; A61K 45/06; A61K 2039/525; A61P 35/00; C12N 15/86

(57) ABSTRACT

Disclosed herein are PSMA and/or STEAP1 polynucleotides, polypeptides, vectors, viruses, vaccines, and vaccine combinations, and their uses.

### CROSS REFERENCE TO RELATED APPLICATIONS

This application claims priority to U.S. Provisional Application No. 63/008,848, filed Apr. 13, 2020, and U.S. Provisional Application No. 63/158,601, filed Mar. 9, 2021, the disclosure of each of which are hereby incorporated by reference in their entirety.

### SEQUENCE LISTING

The instant application contains a Sequence Listing which has been submitted electronically in ASCII format and is hereby incorporated by reference in its entirety. The ASCII copy, created on Mar. 30, 2021, is named 103693.002480 SL.txt and is 64,343 bytes in size.

### FIELD OF THE INVENTION

Provided herein are PSMA and/or STEAP1 polypeptides, polynucleotides encoding the polypeptides, vectors comprising the polynucleotides, viruses comprising the polynucleotides, vaccines, and their uses.

### BACKGROUND OF THE INVENTION

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens such as testosterone promote prostate cancer growth. At its early stages, localized prostate cancer is often curable with local therapy including, for example, surgical removal of the prostate gland and radiotherapy. However, when local therapy fails to cure prostate cancer, as it does in up to a third of men, the disease progresses into incurable metastatic disease (i.e., disease in which the cancer has spread from one part of the body to other parts).

For many years, the established standard of care for men with malignant castration-resistant prostate cancer (mCRPC) was docetaxel chemotherapy. More recently, abiraterone acetate (ZYTIGA®) in combination with prednisone has been approved for treating metastatic castrate resistant prostate cancer. Androgen receptor (AR)-targeted agents, such as enzalutamide (XTANDI®) have also entered the market for treating metastatic castration-resistant prostate cancer. Platinum-based chemotherapy has been tested in a number of clinical studies in molecularly unselected prostate cancer patients with limited results and significant toxicities. However, there remains a subset of patients who either do not respond initially or become refractory (or resistant) to these treatments. No approved therapeutic options are available for such patients.

### BRIEF SUMMARY OF THE INVENTION

Provided herein are polynucleotides encoding PSMA, STEAP1, or a combination of PSMA and STEAP1. Transgenes comprising the polynucleotides are also provided.

Also disclosed are vectors comprising the polynucleotide encoding PSMA, STEAP1, or a combination of PSMA and STEAP1, and cells comprising the disclosed vectors.

Viruses comprising the disclosed polynucleotides are also provided.

Further disclosed herein are vaccines. In some embodiments, the vaccine comprises any of the disclosed polynucleotides encoding PSMA, STEAP1, or a combination of PSMA and STEAP1. In some embodiments, the vaccine comprises a vaccine combination comprising a polynucleotide encoding PSMA, a polynucleotide encoding STEAP1, and a polynucleotide encoding PSMA and a polynucleotide encoding STEAP1.

The disclosure also provides methods of enhancing an immune response against a prostate cancer in subject, and methods of treating a subject afflicted with the prostate cancer, comprising administering to the subject any of the disclosed polynucleotides, transgenes, polypeptides, vectors, cells, viruses, or vaccines.

The methods of enhancing an immune response against a prostate cancer in a subject in need thereof can comprise administering to the subject an immunologically effective amount of a polynucleotide encoding PSMA for priming the immune response, an immunologically effective amount of a polynucleotide encoding STEAP1 for priming the immune response, and an immunologically effective amount of a polynucleotide encoding PSMA and STEAP1 for boosting the immune response.

The methods of treating a subject afflicted with a prostate cancer can comprise administering to the subject an immunologically effective amount of a polynucleotide encoding PSMA for priming the immune response, an immunologically effective amount of a polynucleotide encoding STEAP1 for priming the immune response, and an immunologically effective amount of a polynucleotide encoding PSMA and STEAP1 for boosting the immune response.

### BRIEF DESCRIPTION OF THE DRAWINGS

FIG. 1A and FIG. 1B show representative flow cytometry plots of intracellular cytokine staining (ICS) (TNFα, IFNγ, and IL-2) from a single donor showing antigen-specific CD8⁺ and CD4⁺ T cell recall responses 12 days following antigen priming by APCs transduced with Ad26.hPSMA (Ad26-PSMA in the Figure). Numbers in the gates indicate the percentages of total CD8⁺ (FIG. 1A) or CD4⁺ (FIG. 1B) T cells staining positive for the respective cytokines. No statistical analyses were performed applicable to the displayed data. Ad26-Empty: empty vector.

FIG. 2A and FIG. 2B show representative flow cytometry plots of ICS (TNFα, IFNγ, and IL-2) from a single donor showing antigen-specific CD8⁺ (FIG. 2A) and CD4⁺ (FIG. 2B) T cell recall responses 12 days following antigen priming by APCs transduced with Ad26.hSTEAP1 (Ad26-STEAP1 in the Figure). Numbers in the gates indicate the percentages of total CD8⁺ or CD4⁺ T cells staining positive for the respective cytokines. No statistical analyses were performed applicable to the displayed data. Ad26-Empty: empty vector.

FIG. 3 shows the log 10 of the number of IFNγ spot forming units (SFU) per 10⁶ splenocytes isolated from mice immunized with 10⁹ or 10¹⁰ virus particles (vp) of Ad26.hPSMA, Ad.26.STEAP1, or an empty vector (Ad26-empty) as indicated after stimulation overnight with hPSMA peptide pools. The geometric mean response per group is indicated with a horizontal line. The dotted lines indicate the background of the assay defined as the 95% percentile of SFU observed in non-stimulated splenocytes. IFNγ was measured using ELISpot. For statistical analysis, a Wilcoxon Rank Sum test with Bonferroni correction was used.

FIG. 4 shows the log of the number of IFNγ spot forming units (SFU) per 10⁶ splenocytes from splenocytes isolated from mice immunized with 10⁹ or 10¹⁰ 103693.002480 virus particles (vp) of Ad26.hPSMA, Ad.26.STEAP1, or an empty vector (Ad26-empty) as indicated after stimulation overnight with hSTEAP1 peptide pools. The geometric mean response per group is indicated with a horizontal line. The dotted lines indicate the background of the assay defined as the 95% percentile of SFU observed in non-stimulated splenocytes. IFNγ was measured using ELISpot. For statistical analysis a Wilcoxon Rank Sum test with Bonferroni correction was used.

FIG. 5 shows the percentage (%) of CD8+ spelenocytes producing IFNγ (CD3⁺CD8⁺IFNγ⁺ cells) isolated from mice immunized with 10⁹ or 10¹⁰ virus particles (vp) of Ad26.hPSMA or an empty vector (Ad26-empty) as indicated after stimulation overnight with hPSMA peptide pools. The geometric mean response per group is indicated with a horizontal line. The dotted line shows the background of the assay defined as the mean plus 3× the standard deviation of the background staining, values below this value was set at this cut-off. IFNγ was measured using intracellular cytokine staining (ICS).

FIG. 6 shows the percentage (%) of CD8+ spelenocytes producing IFNγ (CD3⁺CD8⁺IFNγ⁺ cells) isolated from mice immunized with 10⁹ or 10¹⁰ virus particles (vp) of Ad26.hSTEAP1 or an empty vector (Ad26-empty) as indicated after stimulation overnight with hSTEAP1 peptide pools. The geometric mean response per group is indicated with a horizontal line. The dotted line shows the background of the assay defined as the mean plus 3× the standard deviation of the background staining, values below this value was set at this cut-off. IFNγ was measured using intracellular cytokine staining (ICS).

FIG. 7 shows the log 10 of the number of IFNγ spot forming units (SFU) per 10⁶ splenocytes from splenocytes isolated from mice immunized with either 10⁸ or 10⁹ virus particles (vp) of Ad26.hPSMA, 10¹⁰ vp of Ad26.STEAP1, co-administration of 10⁹ vp of Ad26.hPSMA and 10¹⁰ vp of Ad26.STEAP1 (each injected into separate legs; “co-ad” in the figure), or 10⁹ vp of Ad26.hPSMA and 10¹⁰ vp of Ad26.STEAP1 mixed prior to injection and injected into one leg (“bedside mixing” in figure). Indicated is the IFN-γ response after stimulation overnight with hPSMA peptide pools. The geometric mean response per group is indicated with a horizontal line. The dotted lines indicate the background of the assay defined as the 95% percentile of SFU observed in non-stimulated splenocytes. IFNγ was measured using ELISpot. No statistically significant difference was observed between the co-ad and bedside mixing groups (ANOVA, Tobit model).

FIG. 8 shows the non-inferiority analyses demonstrating that the magnitude of PSMA-specific immune response induced with co-ad and bedside mixing are non-inferior to Ad26.PSMA.

FIG. 9 shows the log 10 of the number of IFNγ spot forming units (SFU) per 10⁶ splenocytes from mice immunized with either 10⁸ or 10⁹ virus particles (vp) of Ad26.hPSMA, 10¹⁰ vp of Ad26.STEAP1, co-administration of 10⁹ vp of Ad26.hPSMA and 10¹⁰ vp of Ad26.STEAP1 (each injected into separate legs; “co-ad”), or 10⁹ vp of Ad26.hPSMA and 10¹⁰ vp of Ad26.STEAP1 mixed prior to injection and injected into one leg (“bedside mixing”). Indicated is the IFN-γ response after stimulation overnight with hSTEAP1 peptide pools. The geometric mean response per group is indicated with a horizontal line. The dotted lines indicate the background of the assay defined as the 95% percentile of SFU observed in non-stimulated splenocytes. IFNγ was measured using ELISpot. No statistically significant difference was observed between the co-ad and bedside mixing groups (ANOVA, Tobit model).

FIG. 10 shows the non-inferiority analyses demonstrating that the magnitude of the STEAP1-specific immune response induced with co-ad is non-inferior to Ad26.STEAP1, whereas non-inferiority could not be demonstrated for bedside mixing compared to Ad26.STEAP1.

FIG. 11 shows flow cytometry measuring hPSMA expression on a clonal expansion from CT26 tumor cells transduced with lentivirus particles encoding hPSMA (gray) (CT26-hPSMA low in the Figure) compared to CT26 parental cells that lack hPSMA expression (black).

FIG. 12 shows tumor growth kinetics of parental CT26 or CT26-hPSMA low cells as measured by tumor size (mm³) over time post-tumor implant.

FIG. 13 shows tumor size (mm³) over time post-tumor implant in mice harboring CT26-hPSMA tumors treated with either 10¹⁰ vp of Ad26.Empty vector (gray, solid circle), 10¹⁰ vp of Ad26.Empty vector in combination with 5 mg/kg anti-CTLA-4 antibody (gray, open square), 10¹⁰ vp of Ad26.hPSMA (gray, open triangle), or 10¹⁰ vp of Ad26.hPSMA in combination with 5 mg/kg anti-CTLA-4 antibody (black, solid circle) (n=5 mice per group).

FIG. 14 shows the percentage (%) of IFNγ⁺CD8⁺ T cells of blood-isolated CD8⁺ T cells as assessed using ICS after restimulation with an overlapping peptide pool covering the entire hPSMA protein from mice treated with either 10¹⁰ vp of Ad26.Empty vector (gray, solid circle), 10¹⁰ vp of Ad26.Empty vector in combination with 5 mg/kg anti-CTLA-4 antibody (gray, open square), 10¹⁰ vp of Ad26.hPSMA (gray, open triangle), or 10¹⁰ vp of Ad26.hPSMA in combination with 5 mg/kg anti-CTLA-4 antibody (black, solid circle) (n=10 mice per group). The geometric mean response per group is indicated with a horizontal line. ***p<0.0004 student's t-test.

FIG. 15 shows an exemplary study design of the prime-boost vaccination study of mice utilizing Ad26.hPSMA, Ad26.hSTEAP1 and MVA.hPSMA.hSTEAP1.

FIG. 16 shows the log of the number of IFNγ spot forming units (SFU) per 10⁶ splenocytes from splenocytes isolated from mice immunized with MVA.hPSMA.hSTEAP1 as a prime (Group1; Gr1), Ad26.hPSMA+Ad26.hSTEAP1 as a prime (Group 3, Gr3), Ad26.hPSMA+Ad26.hSTEAP1 as a prime and MVA.hPSMA.hSTEAP1 as a boost (Group 4, Gr4) or with an empty Ad26 vector (Ad26.Empty, Group 10, Gr10) and stimulated overnight with PSMA peptide pool. Group 4 prime-boost regimen significantly potentiated immune responses as measured by increased IFNγ production.

FIG. 17 shows the log of the number of IFNγ spot forming units (SFU) per 10⁶ splenocytes from splenocytes isolated from mice immunized with MVA.hPSMA.hSTEAP1 as a prime (Group1; Gr1), Ad26.hPSMA+Ad26.hSTEAP1 as a prime (Group 3, Gr3), Ad26.hPSMA+Ad26.hSTEAP1 as a prime and MVA.hPSMA.hSTEAP1 as a boost (Group 4, Gr4) or with an empty Ad26 vector (Ad26.Empty, Group 10, Gr10) and stimulated overnight with STEAP1 peptide pool. Group 4 prime-boost regimen significantly potentiated immune responses as measured by increased IFNγ production.

FIG. 18 shows an exemplary non-human primate prime-boost study design.

FIG. 19 shows log of the number of IFNγ spot forming units (SFU) per 10⁶ splenocytes from splenocytes isolated over time as indicated in the figure from cynomolgous macaques primed with Ad26.hPSMA and Ad26.hSTEAP1 and boosted at 4 weeks and at 8 weeks with MVA.hPSMA.hSTEAP1 (Group 1, GO), primed with Ad26.hPSMA and Ad26.hSTEAP1 without receiving boost (Group 2, Gr2), primed with Ad26.hPSMA and Ad26.hSTEAP1 and boosted at 4 weeks and at 8 weeks with MVA.hPSMA.hSTEAP1 and administered ipilimumab IV at both 4 weeks and 8 weeks (Group 3, Gr3), and primed with Ad26.hPSMA and Ad26.hSTEAP1 and boosted at 4 weeks and at 8 weeks with MVA.hPSMA.hSTEAP1 and administered ipilimumab SC at both 4 weeks and 8 weeks (Group 4, Gr4) stimulated overnight with hPSMA and hSTEAP1 peptide pools. The lower dotted line corresponds to the cut-off value of 100 SFU/10⁶ cells, whereas the upper dotted line corresponds to the upper limit of quantification (ULoQ). The error bars indicate standard deviation. The arrows refer to the time of immunization. An ANOVA Tobit model with adjustment for potentially censored values was applied on login-transformed total SFU responses with group as explanatory factor. Statistical analysis was done per time point over the total response comparing Group 1 versus Group 2 (primary analysis, significance is shown by *, corresponding to p<0.005) or comparing Group 1 versus Group 3 or Group 4 (secondary analysis, significance is shown by # for Group 1 versus Group 3, corresponding to p=0.032) at the indicated time points.

FIG. 20 shows the schematic representation of an exemplary self-replicating RNA molecule (replicon) derived from alphavirus replicons, where viral structural genes are replaced by gene of interest under the transcriptional control of a subgenomic promoter (SGP). Conserved sequence elements (CSE) at the 5′ and 3′-end act as promoters for minus-strand and positive-strand RNA transcription. After the replicon is delivered into a cell, the non-structural polyprotein precursor (nsP1234) is translated from in vitro transcribed replicon. nsP1234 is at early stages auto-proteolytically processed to the fragments nsP123 and nsP4, which transcribes negative-stranded copies of the replicon. Later, nsP123 is completely processed to single proteins, which assemble to the (+) strand replicase to transcribe new positive-stranded genomic copies, as well as (+) stranded subgenomic transcripts that code for the gene of interest. Subgenomic RNA as well as new genomic RNA is capped and poly-adenylated. Inactive promoters are dotted arrows; active promoters are lined arrows.

### DETAILED DESCRIPTION OF THE INVENTION

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“Combination” refers to two or more distinct components, such as two or more distinct recombinant viruses.

“Recombinant” refers to polynucleotides, polypeptides, vectors, viruses, and other macromolecules that are prepared, expressed, created, or isolated by recombinant means.

“Transgene” refers to the heterologous nucleic acid that is not naturally present in the vector or virus genome and can be inserted into the vector or the virus genome to generate a recombinant virus.

“PSMA” or “hPSMA” refers to human folate hydrolase (FOL1) and encompasses all isoforms and variants, such as isoforms having amino acid sequences found under GenBank accession numbers NP_001014986.1, NP_001180400.1, NP_001180401.1, NP_001180402.1, NP_001338165.1, and NP_004467.1. “PSMA” refers to both naturally occurring human PSMA and recombinantly expressed human PSMA. When recombinantly expressed, the initiator methionine may be absent from PSMA. “PSMA” also encompasses PSMA without the initiator methionine.

“STEAP1” or “hSTEAP1” refers to human STEAP1 metalloreductase and encompasses all isoforms and variants, such as STEAP1 having an amino acid sequences found under GenBank accession number NP_036581.1. “STEAP1” refers to both naturally occurring human STEAP1 and recombinantly expressed human STEAP1. When recombinantly expressed, the initiator methionine may be absent from STEAP1. “STEAP1” also encompasses STEAP1 without the initiator methionine.

Gene(s) of interest (GOI), as used herein, refers to PSMA, STEAP1, or PSMA and STEAP1.

“Located 5′” refers to a more 5′ orientation of a first polynucleotide element in relation to a second polynucleotide element.

“Located 3′” refers to a more 3′ orientation of a first polynucleotide element in relation to a second polynucleotide element.

“Operator-containing promoter” refers to a promoter operably coupled to a transcription repressor (e.g. repressor) operator sequence, such as TetO or CuO.

“Operably linked” refers to an arrangement of elements, wherein the components so described are configured so as to perform their usual function. A nucleic acid is “operably linked” when it is placed into a functional relationship with another nucleic acid sequence. For example, a promoter is operably linked to one or more transgenes if it affects the transcription of the one or more transgenes. Further, control elements operably linked to a coding sequence are capable of effecting the expression of the coding sequence. The control elements need not be contiguous with the coding sequence, so long as they function to direct the expression thereof.

“CMV promoter” refers to a human or mouse CVM promoter that may encompass a CMV enhancer, promoter, exon 1, and/or intron 1 sequences. An exemplary CMV promoter is a human IE1 CMV promoter that encompasses at least a portion of the 5′ enhancer region and at least portion of exon 1 and may optionally include at least a portion of the first intron. SEQ ID NO: 24 provides an exemplary CMV promoter sequence.

“Vaccinia virus promoter p7.5” refers to vaccinia virus early-late p7.5 promoter comprising the polynucleotide sequence of SEQ ID NO: 1.

“T cell enhancer” (TCE) refers to a polypeptide sequence that, when fused to a downstream polypeptide sequence (e.g. PSMA and/or STEAP1) increases the induction of T cells against the downstream polypeptide sequence in the context of vaccination. Examples of T cell enhancers are an invariant chain sequence or fragment thereof a tissue-type plasminogen activator leader sequence optionally including six additional downstream amino acid residues; a PEST sequence; a cyclin destruction box; an ubiquitination signal; or a SUMOylation signal.

“2A self-cleaving peptide” refers to 2A viral self-cleaving peptides that mediate cleavage of polypeptides during translation in eukaryotic cells.

“Isolated” refers to a homogenous population of molecules (such as synthetic polynucleotides, polypeptides vectors, or viruses), which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99%, or 100% purity.

“Treat,” “treating,” or “treatment” of a disease or disorder such as cancer refers to accomplishing one or more of the following: reducing the severity and/or duration of the disorder, inhibiting worsening of symptoms characteristic of the disorder, limiting or preventing recurrence of the disorder in subjects that have previously had the disorder, or limiting or preventing recurrence of symptoms in subjects that were previously symptomatic for the disorder.

“Prostate cancer” is meant to include all types of cancerous growths within prostate or oncogenic processes, metastatic tissues, or malignantly transformed cells, tissues, or organs, irrespective of histopathology type or stage of invasiveness.

“Prevent,” “preventing,” “prevention,” or “prophylaxis” of a disease or disorder means preventing the occurrence of a disorder in a subject.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“In combination with” means that two or more therapeutic agents are administered to a subject together in a mixture, concurrently as single agents or sequentially as single agents in any order.

“Enhance” or “induce,” when in reference to an immune response, refers to increasing the scale and/or efficiency of an immune response or extending the duration of the immune response. The terms are used interchangeably with “augment.”

“Immune response” refers to any response to a vaccine (such as virus) by the immune system of a vertebrate subject. Exemplary immune responses include local and systemic cellular as well as humoral immunity, such as cytotoxic T lymphocytes (CTL) responses, including antigen-specific induction of CD8⁺ CTLs, helper T-cell responses including T-cell proliferative responses and cytokine production (such as production of IL-2, IFNγ, and TNFα) and B-cell responses including antibody response.

“Immunologically effective amount” or “immunologically effective dose” refers to an amount of a virus sufficient to induce a detectable immune response.

“Variant,” “mutant,” or “altered” refers to a polypeptide or a polynucleotide that differs from a reference polypeptide or a reference polynucleotide by one or more modifications, for example one or more substitutions, insertions, or deletions.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result, or embodiment, “about” means within one standard deviation per the practice in the art, or a range of up to 5%, whichever is larger.

“Prime-boost” refers to a method of treating a subject involving priming a T-cell response with a first vaccine followed by boosting the immune response with a second vaccine. These prime-boost immunizations elicit immune responses of greater height and breadth than can be achieved by priming and boosting with the same vaccine. The priming step initiates memory cells and the boost step expands the memory response. Boosting can occur once or multiple times.

The disclosure provides vaccines, vaccine combinations, polynucleotides, polypeptides, and vectors that can be used to treat subjects afflicted with prostate cancer. The disclosure further provides polynucleotides, polypeptides encoded by the polynucleotides, and vectors that can be used to generate the vaccines and vaccine combinations, which may be used for therapeutic purposes as well as for research use. The vaccines and the vaccine combinations may be used to address scientific questions related to vaccine immune responses and antigen presentation in vivo in animal models such as mouse and non-human primates. The polynucleotides may be used to express the polypeptides and to study their effect on cells in vitro after introducing them into cells. The polypeptides may be used to generate antibodies against them.

### Polynucleotides, Polypeptides, Vectors, Cells

Provided herein are polynucleotides that encode PSMA. In some embodiments, the polynucleotide comprises a sequence having at least 90% sequence identity, at least 95% sequence identity, or at least 99% sequence identity to the sequence of SEQ ID NO: 16. In some embodiments, the polynucleotide comprises the sequence of SEQ ID NO: 16. In some embodiments, the polynucleotide encoding PSMA encodes a polypeptide having at least 90% sequence identity, at least 95% sequence identity, or at least 99% sequence identity to the sequence SEQ ID NO: 15. In some embodiments, the polynucleotide encoding PSMA encodes a polypeptide comprising SEQ ID NO: 15. In some embodiments, the polynucleotide encoding PSMA comprises a sequence having at least 90% sequence identity, at least 95% sequence identity, or at least 99% sequence identity to the sequence of SEQ ID NO: 14. In some embodiments, the polynucleotide comprises the sequence of SEQ ID NO: 14. In some embodiments, the transgene comprises the polynucleotide encoding the polypeptide of SEQ ID NO: 15. In some embodiments, the transgene comprises the polynucleotide of SEQ ID NO: 16.

Provided herein are polynucleotides that encode STEAP1. In some embodiments, the polynucleotide comprises a sequence having at least 90% sequence identity, at least 95% sequence identity, or at least 99% sequence identity to the sequence of SEQ ID NO: 19. In some embodiments, the polynucleotide comprises the sequence of SEQ ID NO: 19. In some embodiments, the polynucleotide encoding STEAP1 encodes a polypeptide having at least 90% sequence identity, at least 95% sequence identity, or at least 99% sequence identity to the sequence of SEQ ID NO: 18. In some embodiments, the polynucleotide encoding STEAP1 encodes a polypeptide comprising the sequence of SEQ ID NO: 18. In some embodiments, the polynucleotide encoding STEAP1 comprises a sequence having at least 90% sequence identity, at least 95% sequence identity, or at least 99% sequence identity to the sequence of SEQ ID NO: 17. In some embodiments, the polynucleotide comprises the sequence of SEQ ID NO: 17.

Also provided herein are polynucleotides that encode PSMA and STEAP1. In some embodiments, the polynucleotide encodes a polypeptide having at least 90% sequence identity, at least 95% sequence identity, or at least 99% sequence identity to the sequence of SEQ ID NO: 12. In some embodiments, the polynucleotide encoding PSMA and STEAP1 encodes a polypeptide comprising the sequence of SEQ ID NO: 12. In some embodiments, the polynucleotide comprises a sequence having at least 90% sequence identity, at least 95% sequence identity, or at least 99% sequence identity to the sequence of SEQ ID NO: 11. In some embodiments, the polynucleotide comprises the sequence of SEQ ID NO: 11.

Disclosed herein are vectors comprising any of the disclosed polynucleotides. Cells comprising any of the disclosed vectors are also provided.

The polynucleotides may be in the form of RNA or in the form of DNA. The RNA or DNA may be obtained by cloning or produced synthetically. The DNA may be double-stranded or single-stranded. Methods of generating polynucleotides are known in the art and include chemical synthesis, enzymatic synthesis (e.g. in vitro transcription), enzymatic or chemical cleavage of a longer precursor, chemical synthesis of smaller fragments of the polynucleotides followed by ligation of the fragments, or known PCR methods. The polynucleotide sequence to be synthesized may be designed with the appropriate codons for the desired amino acid sequence. In general, preferred codons may be selected for the intended host in which the sequence will be used for expression.

Provided herein are PSMA polypeptides. In some embodiments, the PSMA comprises a sequence having at least 90% sequence identity, at least 95% sequence identity, or at least 99% sequence identity to the sequence SEQ ID NO: 15. In some embodiments, the PSMA comprises the sequence of SEQ ID NO: 15.

Provided herein are STEAP1 polypeptides. In some embodiments, the STEAP1 comprises a sequence having at least 90% sequence identity, at least 95% sequence identity, or at least 99% sequence identity to the sequence of SEQ ID NO: 18. In some embodiments, the STEAP1 comprises the sequence of SEQ ID NO: 18.

Also provided herein are PSMA and STEAP1 polypeptides. In some embodiments, the PSMA and STEAP1 comprises a sequence having at least 90% sequence identity, at least 95% sequence identity, or at least 99% sequence identity to the sequence of SEQ ID NO: 12. In some embodiments, the PSMA and STEAP1 comprises the sequence of SEQ ID NO: 12.

Methods of making polypeptides are known in the art and include standard molecular biology techniques for cloning and expression of the polypeptides and chemical synthesis of the polypeptides.

Disclosed herein are vector comprising any of the disclose polynucleotides. The vectors may be generated using known techniques. In some embodiments, the vector is an expression vector. In some embodiments, the vector is a viral vector. The vectors may be utilized to generate recombinant viruses or to express any of the polypeptides disclosed herein.

The vector may be a vector intended for expression of the polynucleotide in any host, such as bacteria, yeast, or a mammal. Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Exemplary vectors are plasmids, cosmids, phages, viral vectors, or artificial chromosomes.

Suitable vectors are known to those of skill in the art; many are commercially available for generating recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

In some embodiments, the vector is a viral vector including, but not limited to adenovirus vectors, adeno-associated virus (AAV) vectors (e.g., AAV type 5 and type 2), alphavirus vectors (e.g., Venezuelan equine encephalitis virus (VEE), Sindbis virus (SIN), Semliki forest virus (SFV), and VEE-SIN chimeras), herpes virus vectors (e.g. vectors derived from cytomegaloviruses, like rhesus cytomegalovirus (RhCMV)), arena virus vectors (e.g. lymphocytic choriomeningitis virus (LCMV) vectors), measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), vesicular stomatitis virus vectors, retrovirus, lentivirus, viral like particles, and bacterial spores.

The disclosure also provides a cell (e.g. a host cell) comprising one or more of the disclosed polynucleotides. The disclosure also provides a cell (e.g. a host cell) comprising one or more of the disclosed vectors. The disclosure also provides a cell (e.g. a host cell) that produces one or more of the disclosed polypeptides. The disclosure also provides a cell (e.g. a host cell) that produces one or more rMVAs of the disclosure. The disclosure also provides a cell (e.g. a host cell) that produces one or more rAds of the disclosure. The disclosure also provides a cell (e.g. a host cell) that produces one or more self-replicating RNAs of the disclosure. “Host cell” refers to a cell into which the polynucleotide, vector, rMVA, or rAd is introduced. It is understood that the term host cell is intended to refer not only to the particular subject cell but to the progeny of such a cell, and to a stable cell line generated from the particular subject cell. Because certain modifications may occur in succeeding generations due to either mutation or environmental influences, such progeny may not be identical to the parent cell but are still included within the scope of the term “host cell” as used herein. Such host cells may be eukaryotic cells, prokaryotic cells, plant cells, or archaeal cells. Escherichia coli, bacilli, such as Bacillus subtilis, and other Enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian, or other animal origins. Mammalian eukaryotic cells include immortalized cell lines such as hybridomas or myeloma cell lines such as SP2/0 (American Type Culture Collection (ATCC), Manassas, Va., CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1SV (Lonza Biologics, Walkersville, Md.), CHO-K1 (ATCC CRL-61), DG44, BHK-21, MDCK, VERO, or 293 cells, Other host cells that may be used are PER.C6, PER.C6 TetR cells, E1-transformed A549 cells, avian cells such as chicken embryonic fibroblasts (CEF) or AGE-1 cells such as AGE1.CR.pIX® cells (See e.g. U.S. Pat. No. 8,940,534).

### Viruses

### Adenoviruses

Provided herein are recombinant adenoviruses (rAd) comprising any of the disclosed polynucleotides or transgenes. In some embodiments, the polynucleotide or transgene comprises a polynucleotide encoding PSMA. In some embodiments, the polynucleotide or transgene comprises a polynucleotide encoding STEAP1. In some embodiments, the polynucleotide or transgene comprises a polynucleotide encoding PSMA and a polynucleotide encoding STEAP1.

In some embodiments, the polynucleotide or transgene further comprises an operator-containing promoter operably linked to the polynucleotide encoding PSMA, the polynucleotide encoding STEAP1, or the polynucleotide encoding PSMA and the polynucleotide encoding STEAP1.

In some embodiments, the operator-containing promoter comprises a CMV promoter and a tetracyclin operon operator (TetO). In some embodiments, the TetO comprises the polynucleotide of SEQ ID NO: 22. In some embodiments, the operator-containing promoter comprises the polynucleotide of SEQ ID NO: 20.

In some embodiments, the transgene further comprises a SV40 pA signal. In some embodiments, the SV40 pA comprises the polynucleotide of SEQ ID NO: 21.

The rAd can comprise a polynucleotide encoding a PSMA. In some embodiments, the rAd comprises a polynucleotide encoding a PSMA that comprises the polynucleotide of SEQ ID NO: 14.

In some embodiments, the rAd comprises a transgene that comprises a polynucleotide encoding the polypeptide of SEQ ID NO: 15. In some embodiments, the rAd comprises a transgene that comprises the polynucleotide of SEQ ID NO: 16.

Also provided are rAds comprising the polynucleotide encoding the polypeptide of SEQ ID NO: 15. Provided are rAds comprising the polynucleotide of SEQ ID NO: 16.

The rAds can comprise a polynucleotide encoding STEAP1. In some embodiments, the rAds comprise a polynucleotide encoding STEAP1 that comprises the polynucleotide of SEQ ID NO: 17. In some embodiments, the rAd comprises the polynucleotide encoding the polypeptide of SEQ ID NO: 18. In some embodiments, the rAd comprises the polynucleotide of SEQ ID NO: 19.

Adenovirus may be derived from human adenovirus (Ad) but also from adenoviruses that infect other species, such as bovine adenovirus (e.g. bovine adenovirus 3, BAdV3), a canine adenovirus (e.g. CAdV2), a porcine adenovirus (e.g. PAdV3 or 5), or great apes, such as Chimpanzee (Pan), Gorilla (Gorilla), Orangutan (Pongo), Bonobo (Pan paniscus) and common chimpanzee (Pan troglodytes). Typically, naturally occurring great ape adenoviruses are isolated from stool samples of the respective great ape.

Human adenoviruses may be derived from various adenovirus serotypes including, for example, from human adenovirus serotypes hAd5, hAd7, hAd11, hAd26, hAd34, hAd35, hAd48, hAd49, or hAd50 (the serotypes are also referred to as Ad5, Ad7, Ad11, Ad26, Ad34, Ad35, Ad48, Ad49 or Ad50).

Great ape adenovirus (GAd) may be derived from various adenovirus serotypes, for example from great ape adenovirus serotypes GAd20, GAd19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, or PanAd3.

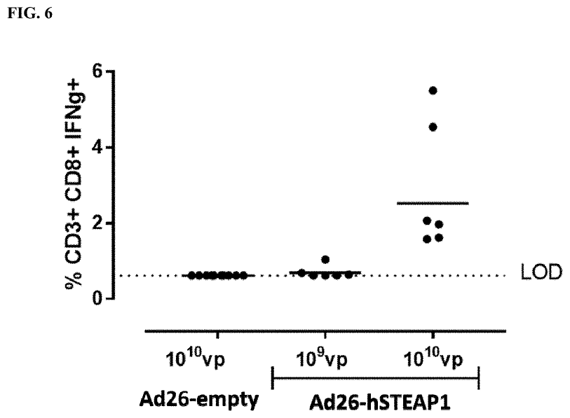

Adenoviruses are known in the art. The sequences of most of the human and non-human adenoviruses are known, and for others can be obtained using routine procedures. An exemplary genome sequence of Ad26 is found in GenBank Accession number EF153474 and in SEQ ID NO: 1 of Int'l Pub. No. WO2007/104792. An exemplary genome sequence of Ad35 is found in FIG. 6 of Int'l Pub. No. WO2000/70071. Ad26 viruses are described for example, in Int'l Pub. No. No. WO2007/104792. Ad35 viruses are described for example in U.S. Pat. No. 7,270,811 and Int'l Pub. No. WO2000/70071. ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63, and ChAd82 viruses are described in Int'l Pub. No. WO2005/071093. PanAd1, PanAd2, PanAd3, ChAd55, ChAd73, ChAd83, ChAd146, and ChAd147 viruses are described in Int'l Pub. No. WO2010/086189.

In some embodiments, the adenovirus is a human adenovirus (Ad). In some embodiments, the Ad is derived from Ad5. In some embodiments, the Ad is derived from Ad11. In some embodiments, the Ad is derived from Ad7. In some embodiments, the Ad is derived from Ad26. In some embodiments, the Ad is derived from Ad34. In some embodiments, the Ad is derived from Ad35. In some embodiments, the Ad is derived from Ad48. In some embodiments, the Ad is derived from Ad49. In some embodiments, the Ad is derived from Ad50.

In some embodiments, the adenovirus is a great ape adenovirus (GAd). In some embodiments, the GAd is derived from GAd20. In some embodiments, the GAd is derived from GAd19. In some embodiments, the GAd is derived from GAd21. In some embodiments, the GAd is derived from GAd25. In some embodiments, the GAd is derived from GAd26. In some embodiments, the GAd is derived from GAd27. In some embodiments, the GAd is derived from GAd28. In some embodiments, the GAd is derived from GAd29. In some embodiments, the GAd is derived from GAd30. In some embodiments, the GAd is derived from GAd31. In some embodiments, the GAd is derived from ChAd3. In some embodiments, the GAd is derived from ChAd4. In some embodiments, the GAd is derived from ChAd5. In some embodiments, the GAd is derived from ChAd6. In some embodiments, the GAd is derived from ChAd7. In some embodiments, the GAd is derived from ChAd8. In some embodiments, the GAd is derived from ChAd9. In some embodiments, the GAd is derived from ChAd9. In some embodiments, the GAd is derived from ChAd10. In some embodiments, the GAd is derived from ChAd11. In some embodiments, the GAd is derived from ChAd16. In some embodiments, the GAd is derived from ChAd17. In some embodiments, the GAd is derived from ChAd19. In some embodiments, the GAd is derived from ChAd20. In some embodiments, the GAd is derived from ChAd22. In some embodiments, the GAd is derived from ChAd24. In some embodiments, the GAd is derived from ChAd26. In some embodiments, the GAd is derived from ChAd30. In some embodiments, the GAd is derived from ChAd31. In some embodiments, the GAd is derived from ChAd32. In some embodiments, the GAd is derived from ChAd31. In some embodiments, the GAd is derived from ChAd33. In some embodiments, the GAd is derived from ChAd37. In some embodiments, the GAd is derived from ChAd38. In some embodiments, the GAd is derived from ChAd44. In some embodiments, the GAd is derived from ChAd55. In some embodiments, the GAd is derived from ChAd63. In some embodiments, the GAd is derived from ChAd68. In some embodiments, the GAd is derived from ChAd73. In some embodiments, the GAd is derived from ChAd82. In some embodiments, the GAd is derived from ChAd83. GAd19-21 and GAd25-31 are described in Int'l Pub. No. WO2019/008111 and represents strains with high immunogenicity and no pre-existing immunity in the general human population. The polynucleotide sequence of GAd20 genome is disclosed in Int'l Pub. No. WO2019/008111.

Recombinant adenoviruses may be derived from various human adenovirus serotypes, for example, from human adenovirus serotypes 5 (hAd5), hAd7, hAd11, hAd26, hAd34, hAd35, hAd48, hAd49, or hAd50. “Ad26” refers to human adenovirus serotype 26. “rAd26” refers to a recombinant adenovirus serotype 26. Other adenovirus and recombinant adenoviruses are named accordingly.

The recombinant adenoviruses of the disclosure are derived from naturally occurring adenoviruses and are typically modified to be replication incompetent, e.g. non-replicating. The recombinant adenoviruses of the disclosure are engineered to comprise at least one functional deletion or a complete removal of a gene product that is essential for viral replication, such as one or more of the adenoviral regions E1, E2, and E4, therefore rendering the adenovirus incapable of replication. The deletion of the E1 region may comprise deletion of EIA, EIB 55K, or EIB 21K, or any combination thereof. Replication deficient recombinant adenoviruses are propagated by providing the proteins encoded by the deleted region(s) in trans by the producer cell by utilizing helper plasmids or engineering the producer cell to express the required proteins. Recombinant adenoviruses may also have a deletion in the E3 region, which is dispensable for replication, and hence such a deletion does not have to be complemented. E3 deletions may be made to facilitate insertion of larger transgenes into the recombinant adenoviruses.

In some embodiments, the recombinant adenovirus comprises a functional deletion or a complete removal of the E1 region and at least part of the E3 region. In some embodiments, the recombinant adenovirus comprises a complete removal of the E1 region (E1 deletion) and a deletion of at least a portion of the E3 region.

In some embodiments, the transgene is inserted into an E1 deletion site. In some embodiments, the transgene is inserted into an E3 deletion site.

The recombinant adenovirus may further comprise a functional deletion or a complete removal of the E4 region and/or the E2 region.

The recombinant adenovirus may be derived from Ad26 or a chimeric Ad26 in which one or more Ad26 capsid proteins (fiber, penton, and hexon) may be derived from a different serotype as long as at least one capsid protein is derived from Ad26. In some cases, the E4-orf6 coding sequence of Ad26 may be replaced with the E4-orf6 of an adenovirus of subgroup C, such as Ad5. This facilitates propagation of the resulting chimeric adenovirus in well-known complementing cell lines that express the E1 genes of Ad5, such as for example 293 cells, PER.C6 cells, and the like (see, e.g. Havenga, et al., 2006, J Gen Virol 87: 2135-43 [61]; WO 03/104467). However, such adenoviruses will not be capable of replicating in non-complementing cells that do not express the E1 genes of Ad5.

Recombinant adenoviruses may be prepared and propagated according to any conventional technique in the field of the art (e.g., Int'l Pub. No. WO1996/17070) using a complementation cell line or a helper virus, which supplies in trans the missing viral genes necessary for viral replication. The cell lines 293 (Graham et al., 1977, J. Gen. Virol. 36: 59-72), PER.C6 (see e.g. U.S. Pat. No. 5,994,128) and E1-transformed A549 cells (Int'l Pub. No. WO1998/39411, U.S. Pat. No. 5,891,690) are commonly used to complement E1 deletions. Other cell lines have been engineered to complement defective vectors (Yeh, et al., 1996, J. Virol. 70: 559-565; Kroughak and Graham, 1995, Human Gene Ther. 6: 1575-1586; Wang, et al., 1995, Gene Ther. 2: 775-783; Lusky, et al., 1998, J. Virol. 72: 2022-203; EP 919627 and Int'l Pub. No. WO1997/04119). Recombinant adenoviruses may be recovered from the culture supernatant but also from the cells after lysis and optionally further purified according to standard techniques (e.g., chromatography, ultracentrifugation, as described in Int'l Pub. No. WO1996/27677, Int'l Pub. No. WO1998/00524, Int'l Pub. No. WO1998/26048 and Int'l Pub. No. WO2000/50573). The construction and methods for propagating adenoviruses are also described in for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913.

Repressor systems may be employed to improve the productivity (such as improved virus rescue and improved yields) and genetic stability (reduced outgrowth of virus mutants with defective transgene) of recombinant adenoviruses (see e.g. U.S. Pat. No. 10,071,151). Transgenes inserted into recombinant adenoviruses expressed under the control of strong constitutive promoters may, depending on the properties of the expressed protein by the transgene, negatively impact production of the recombinant adenovirus (Yoshida and Yamada, 1997, Biochem. Biophys. Res. Commun. 230:426-30; Rubinchik et al., 2000, Gene Ther. 7:875-85; Matthews et al., 1999, J. Gen. Virol. 80:345-53; Edholm et al., 2001, J. Virol. 75:9579-84; Gall et al., 2007, Mol. Biotechnol. 35:263-73). Exemplary repressor systems that may be used are the TetR/TetO (Yao and Eriksson, 1999, Hum. Gene Ther. 10:419-22, EP0990041B1) and the CymR/CuO (Mullick et al., 2006, BMC Biotechnol. 6:43). The transgenes may hence incorporate one of the repressor sequences and the gene of interest may be expressed under the control of TetO or CuO containing strong promoter, such as a CMV promoter. Exemplary sequences that may be used is TetO of SEQ ID NO: 22 and CuO SEQ ID NO: 23. The TetO and CuO sequences may be inserted directly downstream of positions −20 and +7, respectively, of the promoter.

SEQ ID NO: 22 (2×TetO-containing sequence)

### GAGCTCTCCCTATCAGTGATAGAGATCTCCCTATCAGTGATAGAGATCGTCGAC

SEQ ID NO: 23 (CuO-containing sequence)

### AACAAACAGACAATCTGGTCTGTTTGTA

Repression of the transgene expression requires providing the repressor protein TetR or CymR in trans, e.g. in the cell line used to produce the recombinant adenovirus. Cell lines expressing either TetR or CyR may be made by stable transfection of, for example, Per.C6 cells using plasmid pcDNA™6/TR (LifeTechnologies, V1025-20) or a derivative of pcDNA™6/TR in which the TetR-coding sequence is replaced by a codon-optimized CymR-coding sequence. Stable cell lines expressing the TetR or CyR may be generated using known methods and their ability to repress transgene expression during virus replication may be assessed using adenovirus vectors expressing detectable markers such as fluorescent proteins under the repressor-CMV promoter.

The transgene may be inserted into the E1 deletion and/or E3 deletion site in the recombinant adenoviruses in a manner that the transgene does not affect viability of the resultant recombinant adenovirus. The transgene may be inserted into the deleted E1 region or partially deleted E3 region in parallel (transcribed 5′ to 3′) or anti-parallel (transcribed in a 3′ to 5′ direction relative to the vector backbone) orientation. In addition, appropriate transcriptional regulatory elements that are capable of directing expression of the polypeptide encoded by the transgene in the mammalian host cells that the vector is being prepared for use may be operatively linked to the polynucleotide encoding the polypeptide. Operatively linked sequences include both expression control sequences that are contiguous with the nucleic acid sequences that they regulate and regulatory sequences that act in trans, or at a distance to control the regulated nucleic acid sequence.

### Modified Vaccinia Ankara (MVA)

Also provided are recombinant modified vaccinia Ankara (rMVA) viruses comprising any of the disclosed polynucleotides or transgenes. In some embodiments, the polynucleotide or transgene comprises a polynucleotide encoding PSMA. In some embodiments, the polynucleotide or transgene comprises a polynucleotide encoding STEAP1. In some embodiments, the polynucleotide or transgene comprises a polynucleotide encoding PSMA and a polynucleotide encoding STEAP1.

In some embodiments, the polynucleotide or transgene further comprises a poxvirus promoter operably linked to the polynucleotide encoding the PSMA and/or the polynucleotide encoding STEAP1. Suitable poxvirus promoters include, for example, a vaccinia p7.5 promoter, a hybrid early/late promoter, a PrS promoter, a PrSSE promoter, a synthetic or natural early or late promoter, or a cowpox virus ATI promoter. In some embodiments, the poxvirus promoter comprises the vaccinia virus promoter p7.5 comprising the polynucleotide of SEQ ID NO: 1.

The polynucleotide or transgene can further comprise a polynucleotide encoding a first T cell enhancer (TCE) and a polynucleotide encoding a second TCE. In some embodiments, the first TCE and the second TCE comprise a human invariant chain of SEQ ID NO: 25 or a fragment thereof. In some embodiments, the first TCE and the second TCE comprise a mouse invariant chain of SEQ ID NO: 26 or a fragment thereof. In some embodiments, the first TCE and the second TCE comprise a Mandarin fish invariant chain of SEQ ID NO: 27 or a fragment thereof. In some embodiments, the polynucleotide encoding the first TCE encodes the polypeptide of SEQ ID NO: 13 and the polynucleotide encoding the second TCE encodes the polypeptide of SEQ ID NO: 7. In some embodiments, the polynucleotide encoding the first TCE and the polynucleotide encoding the second TCE encode the polypeptide of SEQ ID NO: 29. In some embodiments, the polynucleotide encoding the first TCE comprises the polynucleotide of SEQ ID NO: 2 and/or the polynucleotide encoding the second TCE comprises the polynucleotide of SEQ ID NO: 5.

The polynucleotide or transgene can further comprise a polynucleotide encoding a 2A self-cleaving peptide. In some embodiments, the polynucleotide encoding the 2A self-cleaving peptide encodes the polypeptide of SEQ ID NO: 9. In some embodiments, the polynucleotide encoding the 2A self-cleaving peptide comprises the polynucleotide of SEQ ID NO: 4. In some embodiments, the polynucleotide encoding the 2A self-cleaving peptide encodes the polypeptide of SEQ ID NO: 30. In some embodiments, the polynucleotide encoding the 2A self-cleaving peptide encodes the polypeptide of SEQ ID NO: 31. In some embodiments, the polynucleotide encoding the 2A self-cleaving peptide encodes the polypeptide of SEQ ID NO: 32.

In some embodiments:

the polynucleotide encoding PSMA encodes the polypeptide of SEQ ID NO: 8;

the polynucleotide encoding PSMA comprises the polynucleotide of SEQ ID NO: 3;

the polynucleotide encoding STEAP1 encodes the polypeptide of SEQ ID NO: 10; and/or

the polynucleotide encoding STEAP1 comprises the polynucleotide of SEQ ID NO: 6.

In some embodiments:

the polynucleotide encoding PSMA is located 5′ to the polynucleotide encoding STEAP1;

the poxvirus promoter is located 5′ to the polynucleotide encoding PSMA;

the polynucleotide encoding the first TCE is located 5′ to the polynucleotide encoding PSMA;

the polynucleotide encoding the second TCE is located 3′ to the polynucleotide encoding PSMA; and/or

the polynucleotide encoding the 2A self-cleaving peptide is located 3′ to the polynucleotide encoding PSMA and 5′ to the polynucleotide encoding the second TCE.

In some embodiments, the transgene comprises the polynucleotide encoding the polypeptide of SEQ ID NO: 12. In some embodiments, the transgene comprises the polynucleotide of SEQ ID NO: 11.

The rMVA can be derived from MVA-476 MG/14/78, MVA-572, MVA-574, MVA-575 or MVA-BN. In some embodiments, the rMVA is derived from MVA-476 MG/14/78. In some embodiments, the rMVA is derived from MVA-572. In some embodiments, the rMVA is derived from MVA-574. In some embodiments, the rMVA is derived from MVA-575. In some embodiments, the rMVA is derived from MVA-BN.

The transgene can be inserted into a MVA deletion site I, II, III, IV, V, or VI. In some embodiments, the transgene is inserted into a MVA deletion site III.

Poxviruses (Poxviridae) may be derived from smallpox virus (variola), vaccinia virus, cowpox virus, or monkeypox virus. Exemplary vaccinia viruses include the Copenhagen vaccinia virus (W), New York Attenuated Vaccinia Virus (NYVAC), ALVAC, TROVAC, and Modified Vaccinia Ankara (MVA).

Recombinant MVA (rMVA) virus is an attenuated virus derived from Modified Vaccinia Ankara virus, which is characterized by the loss of its capabilities to reproductively replicate in human cell lines. The recombinant MVA can express any of the disclosed PSMA, STEAP1, or PSMA and STEAP1 polynucleotides, transgenes, or vectors comprising the same.

MVA originates from the dermal vaccinia strain Ankara (Chorioallantois vaccinia Ankara (CVA) virus) that was maintained in the Vaccination Institute, Ankara, Turkey for many years and used as the basis for vaccination of humans. However, due to the often severe post-vaccinal complications associated with vaccinia viruses (VACV), there were several attempts to generate a more attenuated, safer smallpox vaccine.

MVA has been generated by 516 serial passages on chicken embryo fibroblasts of the CVA virus (see Meyer et al., J. Gen. Virol., 72: 1031-1038 (1991) and U.S. Pat. No. 10,035,832). Because of these long-term passages the resulting MVA virus deleted about 31 kilobases of its genomic sequence and, therefore, was described as highly host cell restricted to avian cells (Meyer, H. et al., Mapping of deletions in the genome of the highly attenuated vaccinia virus MVA and their influence on virulence, J. Gen. Virol. 72, 1031-1038, 1991; Meisinger-Henschel et al., Genomic sequence of chorioallantois vaccinia virus Ankara, the ancestor of modified vaccinia virus Ankara, J. Gen. Virol. 88, 3249-3259, 2007.) Comparison of the MVA genome to its parent, CVA, revealed 6 major deletions of genomic DNA (deletion I, II, III, IV, V, and VI), totaling 31,000 basepairs. (Meyer et al., J. Gen. Virol. 72:1031-8 (1991)). It was shown in a variety of animal models that the resulting MVA was significantly avirulent (Mayr, A. & Danner, K. Vaccination against pox diseases under immunosuppressive conditions, Dev. Biol. Stand. 41: 225-34, 1978). Being that many passages were used to attenuate MVA, several different strains or isolates exist, depending on the passage number in CEF cells. Exemplary MVA strains are MVA-572 (deposited at the European Collection of Animal Cell Cultures (“ECACC”), Health Protection Agency, Microbiology Services, Porton Down, Salisbury SP40JG, United Kingdom (“UK”), under the deposit number ECACC V94012707 on Jan. 27, 1994), MVA-575 (deposited at the ECACC under deposit number ECACC V00120707 on Dec. 7, 2000), MVA-Bavarian Nordic (“MVA-BN”) (deposited at the ECACC under deposit number V00080038 on Aug. 30, 2000), VR-1508 (deposited at the American Type Culture collection (ATCC), Manassas, Va., USA), and MVA derived from the virus seed batch 460 MG obtained from 571th passage of Vaccinia Virus on CEF cells, such as MVA 476 MG/14/78 (see e.g. Int'l Pub. No. WO2014/41176).

The transgene can be inserted into a site or region (insertion region) in the MVA genome that does not affect virus viability of the resultant recombinant virus. Such regions can be readily identified by testing segments of virus DNA for regions that allow recombinant formation without seriously affecting virus viability of the recombinant virus. The thymidine kinase (TK) gene is an insertion region that may be used and is present in many viruses, such as in all examined poxvirus genomes. Additionally, MVA contains 6 natural deletion sites, each of which may be used as insertion sites (e.g. deletion I, II, III, IV, V, and VI; see e.g. U.S. Pat. Nos. 5,185,146 and 6,440,442). Further, one or more intergenic regions (IGR) of the MVA may also be used as an insertion site, such as IGRs IGR07/08, IGR 44/45, IGR 64/65, IGR 88/89, IGR 136/137, and IGR 148/149 (see e.g. U.S. Pat. Publ. No. 2018/0064803). Additional suitable insertion sites are described in Int'l Pat. Pub. No. WO2005/048957.

Recombinant MVA is prepared as described in the art using standard molecular biology cloning techniques (Piccini, et al., 1987, Methods of Enzymology 153: 545-563; U.S. Pat. Nos. 4,769,330; 4,772,848; 4,603,112; 5,100,587 and 5,179,993). In an exemplary method, the DNA sequence to be inserted into the virus (e.g. transgene) can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences. rMVA particles may be recovered from the culture supernatant or from the cultured cells after a lysis step (e.g., chemical lysis, freezing/thawing, osmotic shock, sonication, and the like). Consecutive rounds of plaque purification can be used to remove contaminating wild type virus. Viral particles can then be purified using the techniques known in the art (e.g., chromatographic methods or ultracentrifugation on cesium chloride or sucrose gradients).

Optionally, the E. coli plasmid vector can also contain a cassette comprising a marker and/or selection gene operably linked to a poxviral promoter. Suitable markers or selection genes are, e.g., the genes encoding the green fluorescent protein, β-galactosidase, neomycin-phosphoribosyltransferase, or other markers. The use of selection or marker cassettes simplifies the identification and isolation of the generated recombinant MVA. Alternatively, recombinant MVA can be identified by PCR technology.

### Self-Replicating RNA Molecules

The disclosure also provides a Self-replicating RNA encoding PSMA, a STEAP1, or PSMA and STEAP1. In some embodiments, the self-replicating RNA encodes PSMA. In some embodiments, the self-replicating RNA encodes STEAP1. In some embodiments, the self-replicating RNA encodes PSMA and STEAP1.

Self-replicating RNA may be derived from alphavirus. Alphaviruses may belong to the VEEV/EEEV group, or the SF group, or the SIN group. Non-limiting examples of SF group alphaviruses include Semliki Forest virus, O'Nyong-Nyong virus, Ross River virus, Middelburg virus, Chikungunya virus, Barmah Forest virus, Getah virus, Mayaro virus, Sagiyama virus, Bebaru virus, and Una virus. Non-limiting examples of SIN group alphaviruses include Sindbis virus, Girdwood S. A. virus, South African Arbovirus No. 86, Ockelbo virus, Aura virus, Babanki virus, Whataroa virus, and Kyzylagach virus. Non-limiting examples of VEEV/EEEV group alphaviruses include Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), and Una virus (UNAV).

The self-replicating RNA molecules can be derived from alphavirus genomes, meaning that they have some of the structural characteristics of alphavirus genomes, or similar to them. The self-replicating RNA molecules can be derived from modified alphavirus genomes.

Self-replicating RNA molecules may be derived from Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HIV), Fort Morgan virus (FMV), Ndumu (NDUV), and Buggy Creek virus. Virulent and avirulent alphavirus strains are both suitable. In some embodiments, the alphavirus RNA replicon is of a Sindbis virus (SIN), a Semliki Forest virus (SFV), a Ross River virus (RRV), a Venezuelan equine encephalitis virus (VEEV), or an Eastern equine encephalitis virus (EEEV).

In some embodiments, the alphavirus-derived self-replicating RNA molecule is a Venezuelan equine encephalitis virus (VEEV).

The self-replicating RNA molecules can contain RNA sequences from (or amino acid sequences encoded by) a wild-type New World or Old World alphavirus genome. Any of the self-replicating RNA molecules disclosed herein can contain RNA sequences “derived from” or “based on” wild type alphavirus genome sequences, meaning that they have at least 60% or at least 65% or at least 68% or at least 70% or at least 80% or at least 85% or at least 90% or at least 95% or at least 97% or at least 98% or at least 99% or 100% or 80-99% or 90-100% or 95-99% or 95-100% or 97-99% or 98-99% sequence identity with an RNA sequence (which can be a corresponding RNA sequence) from a wild type RNA alphavirus genome, which can be a New World or Old World alphavirus genome.

Self-replicating RNA molecules contain all of the genetic information required for directing their own amplification or self-replication within a permissive cell. To direct their own replication, self-replicating RNA molecules encode polymerase, replicase, or other proteins which may interact with viral or host cell-derived proteins, nucleic acids, or ribonucleoproteins to catalyze the RNA amplification process; and contain cis-acting RNA sequences required for replication and transcription of the replicon-encoded RNA. Thus, RNA replication leads to the production of multiple daughter RNAs. These daughter RNAs, as well as collinear subgenomic transcripts, can be translated to provide in situ expression of a gene of interest, or can be transcribed to provide further transcripts with the same sense as the delivered RNA which are translated to provide in situ expression of the gene of interest. The overall results of this sequence of transcriptions is a huge amplification in the number of the introduced replicon RNAs and so the encoded gene of interest becomes a major polypeptide product of the cells.

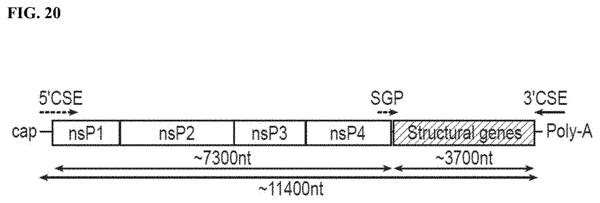

There are two open reading frames (ORF's) in the genome of alphaviruses, non-structural (ns) and structural genes. The ns ORF encodes proteins (nsP1-nsP4) necessary for transcription and replication of viral RNA and are produced as a polyprotein and are the virus replication machinery. The structural ORF encodes three structural proteins: the core nucleocapsid protein C, and the envelope proteins P62 and E1 that associate as a heterodimer. The viral membrane-anchored surface glycoproteins are responsible for receptor recognition and entry into target cells through membrane fusion. The four ns protein genes are encoded by genes in the 5′ two-thirds of the genome, while the three structural proteins are translated from a subgenomic mRNA colinear with the 3′ one-third of the genome. An exemplary depiction of an alphavirus genome is shown in FIG. 20.

Self-replicating RNA molecules can be used as a basis for introducing foreign sequences to host cells by replacing viral sequences encoding structural genes or inserting the foreign sequences 5′ or 3′ of the sequences encoding the structural genes. They can be engineered to replace the viral structural genes downstream of the replicase, which are under control of a subgenomic promoter, by genes of interest (GOI). Upon transfection, the replicase which is translated immediately, interacts with the 5′ and 3′ termini of the genomic RNA, and synthesizes complementary genomic RNA copies. Those act as templates for the synthesis of novel positive-stranded, capped, and poly-adenylated genomic copies, and subgenomic transcripts (FIG. 20). Amplification eventually leads to very high RNA copy numbers of up to 2×10⁵ copies per cell. The result is a uniform and/or enhanced expression of a GOI that can affect vaccine efficacy or therapeutic impact of a treatment. Vaccines based on self-replicating RNA molecules can therefore be dosed at very low levels due to the very high copies of RNA generated compared to conventional viral vector. One of the significant values of the compositions and methods disclosed herein is that vaccine efficacy can be increased in individuals that are in a chronic or acute state of immune activation.

The self-replicating RNA molecules comprising the RNA encoding for the PSMA, STEAP1, or PSMA and STEAP1 polypeptides may be utilized as therapeutics by delivering them to a subject using various technologies, including viral vectors or other delivery technologies as described herein.

The self-replicating RNA molecule can contain all of the genetic information required for directing its own amplification or self-replication within a permissive cell.

The disclosure also provides a self-replicating RNA molecule that can be used as the basis of introducing foreign sequences to host cells (e.g. the PSMA, STEAP1, or PSMA and STEAP1 polypeptides) by replacing viral sequences encoding structural genes.

Provided herein is a self-replicating RNA molecule comprising an RNA sequence derived from any of the polynucleotides of the disclosure.

The self-replicating RNA can encode PSMA. In some embodiments, the self-replicating RNA molecule comprises an RNA sequence derived from a polynucleotide of SEQ ID NO: 16 or having at least 90% sequence identity, or at least 95% sequence identity, or at least 99% sequence identity to SEQ ID NO: 16. In some embodiments, the self-replicating RNA molecule comprises an RNA sequence derived from a polynucleotide of SEQ ID NO: 14 or having at least 90% sequence identity, or at least 95% sequence identity, or at least 99% sequence identity to SEQ ID NO: 14.

The self-replicating RNA can encode STEAP1. In some embodiments, the self-replicating RNA molecule comprises an RNA sequence derived from a polynucleotide of SEQ ID NO: 19 or having at least 90% sequence identity, or at least 95% sequence identity, or at least 99% sequence identity to SEQ ID NO: 19. In some embodiments, the self-replicating RNA molecule comprises an RNA sequence derived from a polynucleotide of SEQ ID NO: 17 or having at least 90% sequence identity, or at least 95% sequence identity, or at least 99% sequence identity to SEQ ID NO: 17.

The self-replicating RNA can encode PSMA and STEAP1. In some embodiments, the self-replicating RNA molecule comprises an RNA sequence encoding an amino acid sequence of SEQ ID NO: 11 or having at least 90% sequence identity, or at least 95% sequence identity, or at least 99% sequence identity to SEQ ID NO: 11.

Any of the above self-replicating RNA molecules can further comprise one or more of the following:
one or more nonstructural genes nsP1, nsP2, nsP3 and nsP4;
at least one of a DLP motif, a 5′ UTR, a 3′UTR and a Poly A; and
a subgenomic promoter.

In some embodiments, for example, the self-replicating RNA molecule can comprise one or more of the following:
one or more nonstructural genes nsP1, nsP2, nsP3 and nsP4;
at least one of a DLP motif, a 5′ UTR, a 3′UTR and a Poly A; and
a subgenomic promoter; and
an RNA encoding for amino acids of SEQ ID NOs: 8 or 10, and operably linked to the subgenomic promoter.

In some embodiments, the self-replicating RNA molecule comprises an RNA sequence encoding a protein or peptide; 5′ and 3′ alphavirus untranslated regions; RNA sequences encoding amino acid sequences derived from New World alphavirus VEEV nonstructural proteins nsP1, nsP2, nsP3 and nsP4; a sub-genomic promoter that is operably linked to and regulates translation of the RNA sequence encoding the protein; a 5′ cap and a 3′ poly-A tail; positive sense, single-stranded RNA; a DLP from Sindbis virus upstream of the non-structural protein 1 (nsP1); a 2A ribosome skipping element; and a nsp1 nucleotide repeat downstream of the 5′-UTR and upstream of the DLP.

In some embodiments, the self-replicating RNA molecules may be at least 1 kb or at least 2 kb or at least 3 kb or at least 4 kb or at least 5 kb or at least 6 kb or at least 7 kb or at least 8 kb or at least 10 kb or at least 12 kb or at least 15 kb or at least 17 kb or at least 19 kb or at least 20 kb in size, or can be 100 bp-8 kb or 500 bp-8 kb or 500 bp-7 kb or 1-7 kb or 1-8 kb or 2-15 kb or 2-20 kb or 5-15 kb or 5-20 kb or 7-15 kb or 7-18 kb or 7-20 kb in size.

Any of the above-disclosed self-replicating RNA molecules can further include a coding sequence for an autoprotease peptide (e.g., autocatalytic self-cleaving peptide), where the coding sequence for the autoprotease is optionally operably linked upstream to the nucleic acid sequence encoding the GOI.

Generally, any proteolytic cleavage site known in the art can be incorporated into the nucleic acid molecules of the disclosure and can be, for example, proteolytic cleavage sequences that are cleaved post-production by a protease. Further suitable proteolytic cleavage sites also include proteolytic cleavage sequences that can be cleaved following addition of an external protease. As used herein the term “autoprotease” refers to a “self-cleaving” peptide that possesses autoproteolytic activity and is capable of cleaving itself from a larger polypeptide moiety. First identified in the foot-and-mouth disease virus (FMDV), a member of the picornavirus group, several autoproteases have been subsequently identified such as, for example, “2A like” peptides from equine rhinitis A virus (E2A), porcine teschovirus-1 (P2A) and Thosea asigna virus (T2A), and their activities in proteolytic cleavage have been shown in various ex vitro and in vivo eukaryotic systems. As such, the concept of autoproteases is available to one of skill in the art as many naturally occurring autoprotease systems have been identified. Well studied autoprotease systems are e.g. viral proteases, developmental proteins (e.g. HetR, Hedgehog proteins), RumA autoprotease domain, UmuD, etc.). Non-limiting examples of autoprotease peptides suitable for the compositions and methods of the present disclosure include the peptide sequences from porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the coding sequence for the autoprotease peptide is operably linked downstream of the DLP motif and upstream to the GOI.

In some embodiments, the autoprotease peptide comprises, or consists of, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the autoprotease peptide includes a peptide sequence of porcine teschovirus-1 2A (P2A).

In some embodiments, the autoprotease peptide is selected from the group consisting of porcine teschovirus-1 2A (P2A), foot-and-mouth disease virus (FMDV) 2A (F2A), Equine Rhinitis A Virus (ERAV) 2A (E2A), Thosea asigna virus 2A (T2A), cytoplasmic polyhedrosis virus 2A (BmCPV2A), Flacherie Virus 2A (BmIFV2A), and a combination thereof.

In some embodiments, the autoprotease peptide is porcine teschovirus-1 2A (P2A).

The incorporation of the P2A peptide in the modified viral RNA replicons allows release of protein encoded by the GOI from the capsid-GOI fusion.

The porcine teschovirus-1 2A (P2A) peptide sequence can be engineered in-frame immediately after the DLP sequence and in-frame immediately upstream of all GOI.

Any of the above-disclosed self-replicating RNA molecules can further include a coding sequence downstream Loop (DLP) motif.

Some viruses have sequences capable of forming one or more stem-loop structures which regulate, for example increase, capsid gene expression. Viral capsid enhancer as used herein refers to a regulatory element comprising sequences capable of forming such stem-loop structures. In some examples, the stem-loop structures are formed by sequences within the coding sequence of a capsid protein and named Downstream Loop (DLP) sequence. As disclosed herein, these stem-loop structures or variants thereof can be used to regulate, for example increase, expression level of genes of interest. For example, these stem-loop structures or variants thereof can be used in a recombinant vector (e.g., in a heterologous viral genome) for enhancing transcription and/or translation of coding sequence operably linked downstream thereto.

Alphavirus replication in host cells is known to induce the double-stranded RNA-dependent protein kinase (PKR). PKR phosphorylates the eukaryotic translation initiation factor 2α (eIF2α). Phosphorylation of eIF2α blocks translation initiation of mRNA and in doing so keeps viruses from a completing a productive replication cycle. Infection of cells with Sindbis virus induces PKR that results in phosphorylation of eIF2α, yet the viral subgenomic mRNA is efficiently translated while translation of all other cellular mRNAs is restricted. The efficient translation of the viral subgenomic mRNA in Sindbis virus is made possible by the presence of a stable RNA hairpin loop (or DLP motif) located downstream of the wild type AUG initiator codon for the virus capsid protein (e.g., capsid enhancer). It has been reported that the DLP structure can stall a ribosome on the wild type AUG and this supports translation of the subgenomic mRNA without the requirement for functional eIF2α. Thus, subgenomic mRNAs of Sindbis virus (SINV) as well as of other alphaviruses are efficiently translated even in cells that have highly active PKR resulting in complete phosphorylation of eIF2α.

The DLP structure was first characterized in Sindbis virus (SINV) 26S mRNA and also detected in Semliki Forest virus (SFV). Similar DLP structures have been reported to be present in at least 14 other members of the Alphavirus genus including New World (for example, MAYV, UNAV, EEEV (NA), EEEV (SA), AURAV) and Old World (SV, SFV, BEBV, RRV, SAG, GETV, MIDV, CHIKV, and ONNV) members. The predicted structures of these Alphavirus 26S mRNAs were constructed based on SHAPE (selective 2′-hydroxyl acylation and primer extension) data (Toribio et al., Nucleic Acids Res. May 19; 44(9):4368-80, (2016); the content of which is hereby incorporated by reference). Stable stem-loop structures were detected in all cases except for CHIKV and ONNV, whereas MAYV and EEEV showed DLPs of lower stability (Toribio et al., 2016 supra). The highest DLP activities were reported for those Alphaviruses that contained the most stable DLP structures.

As an example, members of the Alphavirus genus can resist the activation of antiviral RNA-activated protein kinase (PKR) by means of the downstream loop (DLP) present within viral 26S transcripts, which allows an eIF2-independent translation initiation of these mRNAs. The downstream loop (DLP), is located downstream from the AUG in SINV 26S mRNA and in other members of the Alphavirus genus.

In some embodiments, the disclosed polynucleotides can include a coding sequence for a GOI operably linked to DLP motif(s) and/or the coding sequence for the DLP motifs.

in some, embodiments the self-replicating, RNA molecule comprises a downstream loop (DLP). In some embodiments, the downstream loop (DLP) comprises at least one RNA-stem-loop.

In some instances, DLP activity depends on the distance between the DLP motif and the initiation codon AUG (AUGi). The AUG-DLP spacing in Alphavirus 26S mRNAs is tuned to the topology of the ES6S region of the ribosomal 18S rRNA in a way that allows the placement of the AUGi in the P site of the 40S subunit stalled by the DLP, allowing the incorporation of Met-tRNA without the participation of eIF2. In the case of Sindbis virus, the DLP motif is found in the first roughly 150 nt of the Sindbis subgenomic RNA. The hairpin is located downstream of the Sindbis capsid AUG initiation codon (AUG at nt 50 of the Sindbis subgenomic RNA) and results in stalling a ribosome such that the correct capsid gene AUG is used to initiate translation. Previous studies of sequence comparisons and structural RNA analysis revealed the evolutionary conservation of DLP in SINV and predicted the existence of equivalent DLP structures in many members of the Alphavirus genus (see e.g., Ventoso, J. Virol. 9484-9494, Vol. 86, September 2012).

Without being bound by any particular theory, it is believed that placing the DLP motif upstream of a coding sequence for any GOI typically results in a fusion-protein of N-terminal capsid amino acids that are encoded in the hairpin region to the GOI encoded protein because initiation occurs on the capsid AUG not the GOI AUG.

In some embodiments, the self-replicating RNA molecule comprises a downstream loop placed upstream of the non-structural protein 1 (nsP1). In some embodiments, the downstream loop is placed upstream of the non-structural protein 1 (nsP1) and is joined to the nsP1 by a porcine teschovirus-1 2A (P2A) ribosome skipping element.

The DLP-containing self-replicating RNA can be useful in conferring a resistance to the innate immune system in a subject. Unmodified RNA replicons are sensitive to the initial innate immune system state of cells they are introduced into. If the cells/individuals are in a highly active innate immune system state, the RNA replicon performance (e.g., replication and expression of a GOI) can be negatively impacted. By engineering a DLP to control initiation of protein translation, particularly of non-structural proteins, the impact of the pre-existing activation state of the innate immune system to influence efficient RNA replicon replication is removed or lessened. The result is more uniform and/or enhanced expression of a GOI that can impact vaccine efficacy or therapeutic impact of a treatment.

The DLP motif of the self-replicating RNA can confer efficient mRNA translation in cellular environments where cellular mRNA translation is inhibited. When a DLP is linked with translation of a replicon vector's non-structural protein genes, the replicase and transcriptase proteins are capable of initiating functional replication in PKR activated cellular environments. When a DLP is linked with translation of subgenomic mRNAs, robust GOI expression is possible even when cellular mRNA is restricted due to innate immune activation. Accordingly, engineering self-replicating RNA that contain DLP structures to help drive translation of both non-structural protein genes and subgenomic mRNAs provides a powerful way to overcome innate immune activation.

Examples of a self-replicating RNA molecules comprising a DLP motif are described in U.S. Patent Application Publication US2018/0171340 and Int'l Pub. No. WO2018/106615, the content of which are incorporated herein by reference in their entirety.

Any of the above-disclosed self-replicating RNA molecules can further comprise nonstructural genes nsP1, nsP2, nsP3, and/or nsP4. In some embodiments, the self-replicating RNA molecule does not encode a functional viral structural protein.

Alphavirus genomes encode non-structural proteins nsP1, nsP2, nsP3, and nsP4, which are produced as a single polyprotein precursor, sometimes designated P1234 (or nsP1-4 or nsP1234), and which is cleaved into the mature proteins through proteolytic processing (FIG. 20). nsP1 can be about 60 kDa in size and may have methyltransferase activity and be involved in the viral capping reaction. nsP2 has a size of about 90 kDa and may have helicase and protease activity, while nsP3 is about 60 kDa and contains three domains: a macrodomain, a central (or alphavirus unique) domain, and a hypervariable domain (HVD). nsP4 is about 70 kDa in size and contains the core RNA-dependent RNA polymerase (RdRp) catalytic domain. After infection the alphavirus genomic RNA is translated to yield a P1234 polyprotein, which is cleaved into the individual proteins.

Alphavirus genomes also encode three structural proteins: the core nucleocapsid protein C, and the envelope proteins P62 and E1 that associate as a heterodimer. Structural proteins are under the control of a subgenomic promoter and can be replaced by the GIO.

In some embodiments, the self-replicating RNA can lack (or not contain) the sequence(s) of at least one (or all) of the structural viral proteins (e.g. nucleocapsid protein C, and envelope proteins P62, 6K, and E1). In these embodiments, the sequences encoding one or more structural genes can be substituted with one or more sequences such as, for example, a coding sequence for at least one protein or peptide (e.g. any of the disclosed PSMA, STEAP1, or PSMA and STEAP1 polypeptides) or other polypeptides of interest.

In some embodiments, the self-replicating RNA lacks sequences encoding alphavirus structural proteins; or do not encode alphavirus (or, optionally, any other) structural proteins. In some embodiments, the self-replicating RNA molecules are further devoid of a part or the entire coding region for one or more viral structural proteins. For example, the alphavirus expression system may be devoid of a portion of, or the entire coding sequence for, one or more of the viral capsid proteins C, E1 glycoprotein, E2 glycoprotein, E3 protein, and 6K protein.

In some embodiments, the self-replicating RNA molecule does not contain coding sequences for at least one of the structural viral proteins. In these instances, the sequences encoding structural genes can be substituted with one or more sequences such as, for example, a coding sequence for any of the disclosed PSMA, STEAP1, or PSMA and STEAP1 polynucleotides.

The disclosure also provides a self-replicating RNA molecule comprising nonstructural genes nsP1, nsP2, nsP3, and nsP4, wherein the self-replicating RNA molecule does not encode a functional viral structural protein.

The self-replicating RNA molecule can comprise the coding sequence for at least one, at least two, at least three, or at least four nonstructural viral proteins (e.g. nsP1, nsP2, nsP3, nsP4). The nsP1, nsP2, nsP3, and nsP4 proteins encoded by the replicon are functional or biologically active proteins. In some embodiments, the self-replicating RNA molecule includes the coding sequence for a portion of the at least one nonstructural viral protein. For example, the self-replicating RNA molecules can include about 10%, 20%, 30%, 40%, 50%, 60%, 70%, 80%, 90%, 95%, 100%, or a range between any two of these values, of the coding sequence for the at least one nonstructural viral protein. In some embodiments, the self-replicating RNA molecule can include the coding sequence for a substantial portion of the at least one nonstructural viral protein. As used herein, a “substantial portion” of a nucleic acid sequence encoding a nonstructural viral protein comprises enough of the nucleic acid sequence encoding the nonstructural viral protein to afford putative identification of that protein, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993).

In some embodiments, the self-replicating RNA molecule can include the entire coding sequence for the at least one nonstructural protein. In some embodiments, the self-replicating RNA molecule comprises substantially all the coding sequence for the native viral nonstructural proteins. In certain embodiments, the one or more nonstructural viral proteins are derived from the same virus.

In some embodiments, the downstream loop DLP of the self-replicating RNA molecule placed upstream of the non-structural protein 1 (nsP1) is derived from Sindbis virus.

In some embodiments, the self-replicating RNA molecule comprises nsP1, nsP2, nsP3, and nsP4 sequences derived from the Venezuelan equine encephalitis virus (VEEV) and a DLP motif derived from the Sindbis virus (SIN).

The self-replicating RNA molecules can also have an RNA sub-sequence encoding an amino acid sequence derived from an alphavirus nsP3 macro domain, and an RNA sub-sequence encoding an amino acid sequence derived from an alphavirus nsP3 central domain. The self-replicating RNA molecules can also have an RNA sub-sequence encoding an amino acid sequence derived entirely from an Old World alphavirus nsP3 hypervariable domain, or can have an amino acid sequence having a portion derived from a New World alphavirus nsP3 hypervariable domain and a portion derived from an Old World alphavirus nsP3 hypervariable domain (i.e. the hyper variable domain (HVD) can be a hybrid or chimeric New World/Old World sequence).

In some embodiments, the self-replicating RNA molecules can have an RNA sequence encoding amino acid sequences derived from wild type New World alphavirus nsP1, nsP2, nsP3, and nsP4 protein sequences. In other embodiments, the one or more nonstructural proteins are derived from different viruses.

In some embodiments, the self-replicating RNA molecule may have an RNA sequence encoding an nsP3 macro domain derived from a wild type alphavirus nsP3, and an nsP3 central domain derived from a wild type alphavirus nsP3. In various embodiments, the macro and central domain(s) can both be derived from a New World wild type alphavirus nsP3 or can both be derived from an Old World wild type alphavirus nsP3 protein. In other embodiments, the macro domain can be derived from a New World wild type alphavirus macro domain and the central domain can be derived from an Old World wild type alphavirus central domain, or vice versa. The various domains can be of any sequence described herein.

In some embodiments, the self-replicating RNA molecule contains non VEEV nonstructural proteins nsP1, nsP2, nsP3, and nsP4.

The accumulated experimental evidence has demonstrated that replication/amplification of VEEV and other alphavirus genomes and their defective interfering (DI) RNAs is determined by three promoter elements: (i) the conserved 3′-terminal sequence element (3′ CSE) and the following poly(A) tail; (ii) the 5′ UTR, which functions as a key promoter element for both negative- and positive-strand RNA synthesis; and (iii) the 51-nt conserved sequence element (51-nt CSE), which is located in the nsP1-coding sequence and functions as an enhancer of alphavirus genome replication (Kim et al., PNAS, 2014, 111: 10708-10713, and references therein).

Any of the above-disclosed self-replicating RNA molecules can further include an unmodified 5′ untranslated region (5′UTR).

Previous studies have demonstrated that during VEEV and Sindbis virus infections only a small portion of viral nonstructural proteins (nsPs) is colocalized with dsRNA replication intermediates. Thus, it appears that a large fraction of nsPs are not involved in RNA replication (Gorchakov R, et al. (2008) A new role for ns polyprotein cleavage in Sindbis virus replication. J Virol 82(13):6218-6231). This has provided an opportunity to exploit the under-used ns proteins for amplification of the subgenomic RNAs encoding proteins of interest, which is normally transcribed from the subgenomic promoter and is not further amplified.

In some embodiments, a fragment of the nsP1 of the self-replicating RNA molecule is duplicated downstream of the 5′-UTR and upstream of the DLP. In some embodiments, the first 193 nucleotides of nsP1 are duplicated downstream of the 5′ UTR and upstream of the DLP.

In some embodiments, the self-replicating RNA molecule comprises a modified 5′ untranslated region (5′-UTR). For example, the modified 5′-UTR can comprise one or more nucleotide substitutions at position 1, 2, 4, or a combination thereof. Preferably, the modified 5′-UTR comprises a nucleotide substitution at position 2, more preferably the modified 5′-UTR has a U->G substitution at position 2. Examples of such self-replicating RNA molecules are described in U.S. Patent Application Publication US2018/0104359 and Int'l Pub. No. WO2018/075235, the content of each of which are incorporated herein by reference in their entirety.

In some embodiments, the UTRs can be wild type New World or Old World alphavirus UTR sequences, or a sequence derived from any of them. The 5′ UTR can be of any suitable length, such as about 60 nt or 50-70 nt or 40-80 nt. In some embodiments, the 5′ UTR can also have conserved primary or secondary structures (e.g. one or more stem-loop(s)) and can participate in the replication of alphavirus or of replicon RNA. The 3′ UTR can be up to several hundred nucleotides, for example it can be 50-900 or 100-900 or 50-800 or 100-700 or 200-700 nt. The ‘3 UTR also can have secondary structures, e.g. a step loop, and can be followed by a polyadenylate tract or poly-A tail.

The 5’ and 3′ untranslated regions can be operably linked to any of the other sequences encoded by the replicon. The UTRs can be operably linked to a promoter and/or sequence encoding a protein or peptide by providing sequences and spacing necessary for recognition and transcription of the other encoded sequences.

The GOI (e.g. the PSMA, STEAP1, or PSMA and STEAP1 polynucleotides) can be expressed under the control of a subgenomic promoter. In certain embodiments, instead of the native subgenomic promoter, the subgenomic RNA can be placed under control of internal ribosome entry site (IRES) derived from encephalomyocarditis viruses (EMCV), Bovine Viral Diarrhea Viruses (BVDV), polioviruses, Foot-and-mouth disease viruses (FMD), enterovirus 71, or hepatitis C viruses. Subgenomic promoters range from 24 nucleotides (Sindbis virus) to over 100 nucleotides (Beet necrotic yellow vein virus) and are usually found upstream of the transcription start.

The self-replicating RNA molecules can have a 3′ poly-A tail. The self-replicating RNA molecules can also include a poly-A polymerase recognition sequence (e.g. AAUAAA) near the 3′ end.

In those instances where the self-replicating RNA molecule is to be packaged into a recombinant alphavirus particle, it can contain one or more sequences, so-called packaging signals, which serve to initiate interactions with alphavirus structural proteins that lead to particle formation. In some embodiments, the alphavirus particles comprise RNA derived from one or more alphaviruses, and structural proteins wherein at least one of said structural proteins is derived from two or more alphaviruses.

In some embodiments, the self-replicating RNA molecule comprises a VEEV wherein the structural viral proteins (e.g. nucleocapsid protein C, and envelope proteins P62, 6K, and E1) are removed and replaced by the coding sequence of the PSMA, STEAP1, or PSMA and STEAP1 polypeptides of the disclosure.

### Regulatory Elements

The disclosed polypeptides, transgenes, and vectors may contain one or more regulatory elements operably linked to the polypeptides. The regulatory elements may comprise promoters, enhancers, polyadenylation signals, repressors, and the like.

Some of the commonly used enhancer and promoter sequences in vectors are, for example, hCMV, CAG, SV40, mCMV, EF-1, and hPGK promoters. hCMV promoter may include the hCMV1 IE1 enhancer, promoter, and exon 1 or portion of exon 1. An exemplary sequence of hCMV promoter is provided as SEQ ID NO: 24. Due to its high potency and moderate size of ca. 0.8 kB, the hCMV promoter is commonly used. The hPGK promoter is characterized by a small size (ca. 0.4 kB), but it is less potent than the hCMV promoter. On the other hand, the CAG promoter consisting of a cytomegalovirus early enhancer element, promoter, first exon and intron of chicken beta-actin gene, and splice acceptor of the rabbit beta-globin gene, can direct very potent gene expression that is comparable to the hCMV promoter, but its large size makes it less suitable in viruses where space constraints can be a significant concern, e.g., in adenovirus (AdV), adeno-associated virus (AAV) or lentivirus (LVs).

### SEQ ID NO: 24

TCAATATTGGCCATTAGCCATATTATTCATTGGTTATATAGCATAAATCAATATTGGCTATTGGC CATTGCATACGTTGTATCCATATCATAATATGTACATTTATATTGGCTCATGTCCAACATTACCG CCATGTTGACATTGATTATTGACTAGTTATTAATAGTAATCAATTACGGGGTCATTAGTTCATAG CCCATATATGGAGTTCCGCGTTACATAACTTACGGTAAATGGCCCGCCTGGCTGACCGCCCAACG ACCCCCGCCCATTGACGTCAATAATGACGTATGTTCCCATAGTAACGCCAATAGGGACTTTCCAT TGACGTCAATGGGTGGAGTATTTACGGTAAACTGCCCACTTGGCAGTACATCAAGTGTATCATAT GCCAAGTACGCCCCCTATTGACGTCAATGACGGTAAATGGCCCGCCTGGCATTATGCCCAGTACA TGACCTTATGGGACTTTCCTACTTGGCAGTACATCTACGTATTAGTCATCGCTATTACCATGGTG ATGCGGTTTTGGCAGTACATCAATGGGCGTGGATAGCGGTTTGACTCACGGGGATTTCCAAGTCT CCACCCCATTGACGTCAATGGGAGTTTGTTTTGGCACCAAAATCAACGGGACTTTCCAAAATGTC GTAACAACTCCGCCCCATTGACGCAAATGGGCGGTAGGCGTGTACGGTGGGAGGTCTATATAAGC AGAGCTCGTTTAGTGAACCGTCAGATCGCCTGGAGACGCCATCCACGCTGTTTTGACCTCCATAG AAGACACCGGGACCGATCCAGCCTCCGCGGCCGGGAACGGTGCATTGGA

Additional promoters that may be used are Aotine Herpesvirus 1 major immediate early promoter (AoHV-1 promoter) described in Int'l Pub. No. WO2018146205A1.

Other promoters that may be used are one or more poxvirus promoters. Exemplary poxvirus promoters are a p7.5 promoter, a hybrid early/late promoter, or a PrS promoter, a PrS5E promoter, a synthetic or natural early or late promoter, or a cowpox virus ATI promoter. The poxvirus promoters may be used to drive transgene expression in recombinant MVA.

The promoter may be operably coupled to a repressor operator sequence, to which a repressor protein can bind in order to repress expression of the promoter in the presence of the repressor protein.

In some embodiments, the repressor operator sequence is a TetO sequence or a CuO sequence (see e.g. U.S. Pat. No. 9,790,256) and as described herein.

In certain cases, it may be desirable to express at least two separate polypeptides from the same vector. In this case each polynucleotide may be operably linked to the same or different promoter and/or enhancer sequences, or well-known bicistronic expression systems for example by utilizing internal ribosome entry site (IRES) from encephalomyocarditis virus may be used. Alternatively, bidirectional synthetic promoters may be used, such as a hCMV-rhCMV promoter and other promoters described in Int'l Pub. No. WO2017220499A1.

Polyadenylation signals may be derived from SV40 or bovine growth hormone (BGH).

The disclosed polynucleotides and transgenes may also contain one or more polynucleotides encoding one or more T cell enhancers (TCE). TCEs are polypeptide sequence that enhance immunogenicity of proteins they are fused with, such as PSMA and/or STEAP1. TCE may be fused to a N-terminus or a C-terminus of the protein. Exemplary T cell enhancers are an invariant chain sequence or fragment thereof of human (SEQ ID NO: 25), mouse (SEQ ID NO: 26) or Mandarin fish (SEQ ID NO: 27); a fragment of the invariant chain, such as a fragment of a Mandarin fish invariant chain having the amino acid sequence of SEQ ID NO: 7 or SEQ ID NO: 28 or a tissue-type plasminogen activator leader sequence optionally including six additional downstream amino acid residues (SEQ ID NO: 29). Other exemplary TCEs include a PEST sequence; a cyclin destruction box; an ubiquitination signal or a SU29MOylation signal.

Human invariant chain
(SEQ ID NO: 25)
MHRRRSRSCREDQKPVMDDQRDLISNNEQLPMLGRRPGAPESKCSRGALY
TGFSILVTLLLAGQATTAYFLYQQQGRLDKLTVTSQNLQLENLRMKLPKP
PKPVSKMRMATPLLMQALPMGALPQGPMQNATKYGNMTEDHVMHLLQNAD
PLKVYPPLKGSFPENLRHLKNTMETIDWKVFESWMHHWLLFEMSRHSLEQ
KPTDAPPKESLELEDPSSGLGVTKQDLGPVPM
Mouse invariant chain
(SEQ ID NO: 26)
MDDQRDLISNHEQLPILGNRPREPERCSRGALYTGVSVLVALLLAGQATT
AYFLYQQQGRLDKLTITSQNLQLESLRMKLPKSAKPVSQMRMATPLLMRP
MSMDNMLLGPVKNVTKYGNMTQDHVMHLLTRSGPLEYPQLKGTFPENLKH
LKNSMDGVNWKIFESWMKQWLLFEMSKNSLEEKKPTEAPPKEPLDMEDLS
SGLGVTRQELGQVTL
Mandarin fish invariant chain
(SEQ ID NO: 27)
MADSAEDAPMARGSLAGSDEALILPAGPTGGSNSRALKVAGLTTLTCLLL
ASQVFTAYMVEGQKEQIHTLQKNSERMSKQLTRSSQAVAPMKMHMPMNSL
PLLMDFTPNEDSKTPLTKLQDTAVVSVEKQLKDLMQDSQLPQFNETFLAN
LQGLKQQMNESEWKSFESWMRYWLIFQMAQQKPVPPTADPASLIKTKCQM
ESAPGVSKIGSYKPQCDEQGRYKPMQCWHATGFCWCVDETGAVIEGTTMR
GRPDCQRRALAPRRMAFAPSLMQKTISIDDQ
SEQ ID NO: 7
(fragment of Mandarin fish invariant chain)
GQKEQIHTLQKNSERMSKQLTRSSQAV
SEQ ID NO: 13
(fragment of Mandarin fish invariant chain)
MGQKEQIHTLQKNSERMSKQLTRSSQAV
SEQ ID NO: 28
(fragment of Mandarin fish invariant chain)
QIHTLQKNSERMSKQL
SEQ ID NO: 29
TPA
MDAMKRGLCCVLLLCGAVFVSPSQEIHAR

The polynucleotides or transgenes may also contain one or more polynucleotides encoding one or more 2A self-cleaving peptides. 2A self-cleaving peptides are viral or synthetic short peptides that mediate cleavage of polypeptides during translation. 2A peptides have been identified, for example, from foot- and mouth disease virus (F2), equine rhinitis A virus (E2A), porcine teschevirus-1 (P2A), and Thosea asigna virus 2A (T2A). The mechanism of 2A-mediated self-cleavage was recently discovered to be ribosome skipping and a highly conserved sequence GDVEXNPGP (SEQ ID NO: 33) shared by different 2As at the C-terminus was found essential for the self-cleavage. Any 2A self-cleaving sequence can be introduced into the polynucleotides or transgenes between the polynucleotide encoding PSMA and the polynucleotide encoding STEAP1. Amino acid sequences of exemplary 2A self-cleaving peptides are P2A (SEQ ID NO: 30), T2A (SEQ ID NO: 31), E2A (SEQ ID NO: 32), and F2A (SEQ ID NO: 9).

P2A
(SEQ ID NO: 30)
GSGATNFSLLKQAGDVEENPGP
T2A
(SEQ ID NO: 31)
GSGEGRGSLLTCGDVEENPGP
E2A
(SEQ ID NO: 32)
GSGQCTNYALLKLAGDVESNPGP
F2A
(SEQ ID NO: 9)
APVKQTLNFDLLKLAGDVESNPGP

### Vaccines and Vaccine Combinations

Provided herein are vaccines comprising a polynucleotide or transgene encoding PSMA. Also provided herein are vaccines comprising a polynucleotide or transgene encoding STEAP1. Further provided herein are vaccines comprising a polynucleotide or transgene encoding PSMA and a polynucleotide encoding STEAP1.

Vaccine combinations comprising two or more of the disclosed vaccines are also provided. In some embodiments, the vaccine combination comprises:

a first polynucleotide or transgene encoding PSMA;

a second polynucleotide or transgene encoding STEAP1; and

a third polynucleotide or transgene encoding PSMA and STEAP1.

In some embodiments, a recombinant Ad26 virus comprises the first and second polynucleotides or transgenes. In some embodiments, an MVA virus comprises the first and second polynucleotides or transgenes. In some embodiments, a self-replicating RNA comprises the first and second polynucleotides or transgenes.

In some embodiments, a recombinant Ad26 virus comprises the third polynucleotide or transgene. In some embodiments, an MVA virus comprises the third polynucleotide or transgene. In some embodiments, a self-replicating RNA comprises the third polynucleotide or transgene.

Also disclosed herein are vaccine combinations, comprising:

a first recombinant Ad26 virus comprising a first polynucleotide or transgene encoding PSMA;

a second recombinant Ad26 virus comprising a second polynucleotide or transgene encoding STEAP1; and

a recombinant MVA virus comprising a third polynucleotide or transgene encoding STEAP1.

Also disclosed herein are vaccine combinations, comprising: a first recombinant Ad26 virus comprising a first polynucleotide or transgene encoding PSMA;

a second recombinant Ad26 virus comprising a second polynucleotide or transgene encoding STEAP1; and

a self-replicating RNA comprising a third polynucleotide or transgene encoding PSMA and STEAP1.

Also provided herein are vaccine combinations, comprising:

a first recombinant MVA virus comprising a first polynucleotide or transgene encoding PSMA;

a second recombinant MVA virus comprising a second polynucleotide or transgene encoding STEAP1; and

a recombinant Ad26 virus comprising a third polynucleotide or transgene encoding PSMA and STEAP1.

Disclosed herein are vaccine combinations, comprising:

a first recombinant MVA virus comprising a first polynucleotide or transgene encoding PSMA;

a second recombinant MVA virus comprising a second polynucleotide or transgene encoding STEAP1; and

a self-replicating RNA comprising a third polynucleotide or transgene encoding PSMA and STEAP1.

Disclosed herein are vaccine combinations, comprising:

a self-replicating RNA comprising a first polynucleotide or transgene encoding PSMA;

a self-replicating RNA comprising a second polynucleotide or transgene encoding STEAP1; and

a recombinant Ad26 virus comprising a third polynucleotide or transgene encoding PSMA and STEAP1.

Also provided herein are vaccine combinations, comprising: a self-replicating RNA comprising a first polynucleotide or transgene encoding PSMA;

a self-replicating RNA comprising a second polynucleotide or transgene encoding STEAP1; and

a recombinant MVA virus comprising a third polynucleotide or transgene encoding PSMA and STEAP1.

In some embodiments, the first polynucleotide or transgene and the second polynucleotide or transgene further comprise an operator-containing promoter operably linked to the PSMA polynucleotide sequence and the STEAP1 polynucleotide sequence. In some embodiments, the operator-containing promoter comprises a CMV promoter and a tetracyclin operon operator (TetO). In some embodiments, the operator-containing promoter comprises the polynucleotide of SEQ ID NO: 20.

In some embodiments, the first polynucleotide or transgene and the second polynucleotide or transgene further comprise a SV40 polyadenylation signal (SV40 pA).

In some embodiments, the first polynucleotide or transgene that encodes PSMA encodes the polypeptide of SEQ ID NO: 15. In some embodiments, the first polynucleotide or transgene that encodes PSMA comprises the polynucleotide sequence of SEQ ID NO: 14.

In some embodiments, the first polynucleotide or transgene comprises the polynucleotide encoding the polypeptide of SEQ ID NO: 15. In some embodiments, the first polynucleotide or transgene comprises the polynucleotide of SEQ ID NO: 16.

In some embodiments, in the second polynucleotide or transgene that encodes STEAP1 encodes a polypeptide of SEQ ID NO: 18. In some embodiments, the second polynucleotide or transgene that encodes STEAP1 comprises the polynucleotide of SEQ ID NO: 17.

In some embodiments, in the second polynucleotide or transgene comprises the polynucleotide encoding the polypeptide of SEQ ID NO: 18. In some embodiments, the first polynucleotide or transgene comprises the polynucleotide of SEQ ID NO: 19.

In some embodiments, in first polynucleotide or transgene and the second polynucleotide or transgene are inserted into rAd26 E1 deletion site.

In some embodiments, the third polynucleotide or transgene further comprises a poxvirus promoter operably linked to the polynucleotide encoding the PSMA and the polynucleotide encoding STEAP1. In some embodiments, the poxvirus promoter comprises a vaccinia virus promoter p7.5 of SEQ ID NO: 1.

In some embodiments, in the third polynucleotide or transgene further comprises a polynucleotide encoding a first T cell enhancer (TCE) and a polynucleotide encoding a second TCE. In some embodiments, the first TCE and the second TCE comprise a human invariant chain of SEQ ID NO: 25 or a fragment thereof. In some embodiments, the first TCE and the second TCE comprise a mouse invariant chain of SEQ ID NO: 26 or a fragment thereof. In some embodiments, the first TCE and the second TCE comprise a Mandarin fish invariant chain of SEQ ID NO: 27 or a fragment thereof. In some embodiments, the polynucleotide encoding the first TCE encode the polypeptide of SEQ ID NO: 13 and the polynucleotide encoding the second TCE encode the polypeptide of SEQ ID NO: 7. In some embodiments, the polynucleotide encoding the first TCE and the polynucleotide encoding the second TCE encode the polypeptide of SEQ ID NO: 29. In some embodiments, the polynucleotide encoding the first TCE comprises the polynucleotide of SEQ ID NO: 2 and/or the polynucleotide encoding the second TCE comprises the polynucleotide of SEQ ID NO: 5.

In some embodiments, in the third polynucleotide or transgene further comprises a polynucleotide encoding a 2A self-cleaving peptide. In some embodiments, the third polynucleotide or transgene further comprises a polynucleotide encoding a 2A self-cleaving peptide. In some embodiments, the polynucleotide encoding the 2A self-cleaving peptide encodes the polypeptide of SEQ ID NO: 9. In some embodiments, the polynucleotide encoding the 2A self-cleaving peptide comprises the polynucleotide of SEQ ID NO: 4. In some embodiments, the polynucleotide encoding the 2A self-cleaving peptide encodes the polypeptide of SEQ ID NO: 30. In some embodiments, the polynucleotide encoding the 2A self-cleaving peptide encodes the polypeptide of SEQ ID NO: 31. In some embodiments, the polynucleotide encoding the 2A self-cleaving peptide encodes the polypeptide of SEQ ID NO: 32.

In some embodiments, the third polynucleotide or transgene comprises:
a polynucleotide encoding PSMA that encodes the polypeptide of SEQ ID NO: 8;
a polynucleotide encoding PSMA that comprises the polynucleotide of SEQ ID NO: 3;
a polynucleotide encoding STEAP1 that encodes the polypeptide of SEQ ID NO: 10; and/or
a polynucleotide encoding STEAP1 that comprises the polynucleotide of SEQ ID NO: 6.

In some embodiments, in the third polynucleotide or transgene: the polynucleotide encoding PSMA that is located 5′ to the polynucleotide encoding STEAP1; the poxvirus promoter is located 5′ to the polynucleotide encoding PSMA; the polynucleotide encoding the first TCE is located 5′ to the polynucleotide encoding PSMA; the polynucleotide encoding the second TCE is located 3′ to the polynucleotide encoding PSMA; and/or the polynucleotide encoding the 2A self-cleaving peptide is located 3′ to the polynucleotide encoding PSMA and 5′ to the polynucleotide encoding the second TCE.

In some embodiments, the third polynucleotide or transgene comprises the polynucleotide encoding the polypeptide of SEQ ID NO: 12. In some embodiments, the third polynucleotide or transgene comprises the polynucleotide of SEQ ID NO: 11.

In some embodiments, the rMVA is derived from a virus seed MVA-476 MG/14/78, MVA-572, MVA-574 or MVA-575 or MVA-BN. In some embodiments, the third polynucleotide or transgene is inserted into a rMVA deletion site III.

### Pharmaceutical Compositions

The disclosed vaccines or vaccine combinations may comprise or may be formulated into a pharmaceutical composition comprising the vaccine and a pharmaceutically acceptable excipient. “Pharmaceutically acceptable” refers to the excipient that at the dosages and concentrations employed, will not cause unwanted or harmful effects in the subjects to which they are administered and include carrier, buffer, stabilizer, or other materials well known to those skilled in the art. The precise nature of the carrier or other material may depend on the route of administration, e.g., intramuscular, subcutaneous, oral, intravenous, cutaneous, intramucosal (e.g., gut), intranasal, or intraperitoneal routes. Liquid carriers such as water, petroleum, animal or vegetable oils, mineral oil or synthetic oil may be included. Physiological saline solution, dextrose or other saccharide solution or glycols such as ethylene glycol, propylene glycol, or polyethylene glycol may be included. Exemplary viral formulation are the Adenovirus World Standard (Hoganson et al, 2002): 20 mM Tris pH 8, 25 mM NaCl, 2.5% glycerol; or 20 mM Tris, 2 mM MgCl₂, 25 mM NaCl, sucrose 10% w/v, polysorbate-80 0.02% w/v; or 10-25 mM citrate buffer pH 5.9-6.2, 4-6% (w/w) hydroxypropyl-beta-cyclodextrin (HBCD), 70-100 mM NaCl, 0.018-0.035% (w/w) polysorbate-80, and optionally 0.3-0.45% (w/w) ethanol. Another exemplary viral formulation is 10 mM Tris, 140 mM NaCl at a pH of 7.7. Other buffers can be used, and examples of suitable formulations for the storage and for pharmaceutical administration of purified pharmaceutical preparations are known.

The vaccine may comprise one or more adjuvants. Suitable adjuvants include QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59. Other adjuvants that may be used include lectins, growth factors, cytokines and lymphokines such as alpha-interferon, gamma interferon, platelet derived growth factor (PDGF), granulocyte-colony stimulating factor (gCSF), granulocyte macrophage colony stimulating factor (gMCSF), tumor necrosis factor (TNF), epidermal growth factor (EGF), IL-1, IL-2, IL-4, IL-6, IL-8, IL-10, IL-12 or TLR agonists.

The terms “adjuvant” and “immune stimulant” are used interchangeably herein and are defined as one or more substances that cause stimulation of the immune system. In this context, an adjuvant is used to enhance an immune response to the viral vectors described herein.

The pharmaceutical composition may in certain embodiments be the vaccine.

### Kits

The disclosure also provides a kit comprising the vaccine or vaccine combination of the disclosure.

The disclosure also provides a kit comprising the rMVA. The disclosure also provides a kit comprising the rAd26. The kits may be used to facilitate performing the methods described herein. In some embodiments, the kit further comprises reagents to facilitate entry of the vaccines into a cell, such as lipid-based formulations or viral packaging materials.

### Methods of Treatment, Uses, and Administration

The polynucleotides, polypeptides, vectors, viruses, vaccines, and vaccine combinations may be used as research tools and for therapeutic purposes, such as in the treatment of prostate cancer.

Provided herein are methods of preventing or treating a prostate cancer in a subject, comprising administering to the subject a therapeutically effective amount of any one of the disclosed polynucleotides, polypeptides, vectors, viruses, vaccines, and vaccine combinations.

Also provided are methods of enhancing an immune response against a prostate cancer in a subject afflicted with the prostate cancer, comprising administering to the subject any one of the disclosed polynucleotides, polypeptides, vectors, viruses, vaccines, and vaccine combinations.

Provided herein are uses of the disclosed polynucleotides, polypeptides, vectors, viruses, vaccines, and vaccine combinations in the preparation of a medicament for preventing or treating a prostate cancer in a subject, comprising administering to the subject a therapeutically effective amount of any one of the disclosed polynucleotides, polypeptides, vectors, viruses, vaccines, and vaccine combinations.

Also provided are uses of the disclosed polynucleotides, polypeptides, vectors, viruses, vaccines, and vaccine combinations in the preparation of a medicament for enhancing an immune response against a prostate cancer in a subject afflicted with the prostate cancer, comprising administering to the subject any one of the disclosed polynucleotides, polypeptides, vectors, viruses, vaccines, and vaccine combinations.

In some embodiments, the methods of enhancing an immune response against a prostate cancer in a subject in need thereof comprise administering to the subject:

an immunologically effective amount of a first polynucleotide or transgene encoding PSMA for priming the immune response;

an immunologically effective amount of a second polynucleotide or transgene encoding STEAP1 for further priming the immune response; and

an immunologically effective amount of a third polynucleotide or transgene encoding PSMA and STEAP1 for boosting the immune response.

In some embodiments, the methods of treating a subject afflicted with a prostate cancer comprise administering to the subject:

an immunologically effective amount of a first polynucleotide or transgene encoding PSMA for priming the immune response;

an immunologically effective amount of a second polynucleotide or transgene encoding STEAP1 for further priming the immune response; and

an immunologically effective amount of a third polynucleotide or transgene encoding PSMA and STEAP1 for boosting the immune response.

In some embodiments of the disclosed methods:

a first recombinant Ad26 virus comprises the first polynucleotide or transgene encoding PSMA;

a second recombinant Ad26 virus comprises the second polynucleotide or transgene encoding STEAP1; and

a recombinant MVA virus comprises the third polynucleotide or transgene encoding PSMA and STEAP1.

In some embodiments of the disclosed methods:

a first recombinant Ad26 virus comprises the first polynucleotide or transgene encoding PSMA;

a second recombinant Ad26 virus comprises the second polynucleotide or transgene encoding STEAP1; and

a self-replicating RNA comprises the third polynucleotide or transgene encoding PSMA and STEAP1.

In some embodiments of the disclosed methods:

a first recombinant MVA virus comprises the first polynucleotide or transgene encoding PSMA;

a second recombinant MVA virus comprises the second polynucleotide or transgene encoding STEAP1; and

a recombinant Ad26 virus comprises the third polynucleotide or transgene encoding PSMA and STEAP1.

In some embodiments of the disclosed methods: a first recombinant MVA virus comprises the first polynucleotide or transgene encoding PSMA;

a second recombinant MVA virus comprises the second polynucleotide or transgene encoding STEAP1; and

a self-replicating RNA comprises the third polynucleotide or transgene encoding PSMA and STEAP1.

In some embodiments of the disclosed methods:

a self-replicating RNA comprises the first polynucleotide or transgene encoding PSMA;

a self-replicating RNA comprises the second polynucleotide or transgene encoding STEAP1; and

a recombinant Ad26 virus comprises the third polynucleotide or transgene encoding PSMA and STEAP1.

In some embodiments of the disclosed methods:

a self-replicating RNA comprises the first polynucleotide or transgene encoding PSMA;

a self-replicating RNA comprises the second polynucleotide or transgene encoding STEAP1; and

a recombinant MVA virus comprises the third polynucleotide or transgene encoding PSMA and STEAP1.

In some embodiments, the Ad26, the MVA, and/or the self-replicating RNA are formulated in a pharmaceutical composition.

In some embodiments, the immune response is a CD8+ T cell response or a CD4+ T cell response.

In some embodiments, the first recombinant Ad26 virus comprises rAd26.PSMA, the second recombinant Ad26 virus comprises rAD26.STEAP1, and the recombinant MVA virus comprises rMVA.PSMA.STEAP1.

In some embodiments, the prostate cancer is an adenocarcinoma. In some embodiments, the prostate cancer is a metastatic prostate cancer. In some embodiments, the prostate cancer has metastasized to rectum, lymph node, or bone, or any combination thereof.

In some embodiments, the prostate cancer is a relapsed or a refractory prostate cancer. In some embodiments, the prostate cancer is a castration resistant prostate cancer. In some embodiments, the prostate cancer is sensitive to an androgen deprivation therapy. In some embodiments, the prostate cancer is insensitive to the androgen deprivation therapy. Androgen deprivation therapies include abiraterone, ketoconazole, enzalutamide, galeterone, ARN-509, and orteronel (TAK-700).

The polynucleotides, polypeptides, vectors, viruses, vaccines, and vaccine combinations may be administered by intramuscular or subcutaneous injection. However other modes of administration such as intravenous, cutaneous, intradermal, or nasal can be envisaged as well. Intramuscular administration of the vaccines can be achieved by using a needle. An alternative is the use of a needleless injection device to administer the composition (using, e.g., Biojector™) or a freeze-dried powder containing the vaccine.

For intravenous, cutaneous, or subcutaneous injection, or injection at the site of affliction, the vector may be the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation may also be employed.

Typically, administration will have a prophylactic aim to generate an immune response against the prostate neoantigens (i.e. PSMA and/or STEAP1) before development of symptoms of prostate cancer.

The polynucleotides, polypeptides, vectors, viruses, vaccines, and vaccine combinations are administered to a subject, giving rise to an immune response in the subject. The polynucleotides, polypeptides, vectors, viruses, vaccines, and vaccine combinations may induce a humoral as well as a cell-mediated immune response. In a typical embodiment the immune response is a protective immune response.

The actual amount administered, and rate and time-course of administration, will depend on the nature and severity of the condition being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

In one exemplary regimen, recombinant Ad26.PSMA virus and recombinant Ad26.STEAP1 virus are administered (e.g., intramuscularly) in a volume ranging between about 100 μL to about 10 ml containing concentrations of about 10⁴ to 10¹² virus particles/ml. rAd26.PSMA and rAd26.STEAP1 may be administered in a volume ranging between 0.25 and 1.0 ml, such as in a volume of 0.5 ml.

The recombinant adenovirus virus may be administered in an amount of about 10⁹ to about 10¹² viral particles (vp) to a human subject during one administration, more typically in an amount of about 10¹⁰ to about 10¹² vp.

The recombinant MVA virus may be administered (e.g., intramuscularly) in a volume ranging between about 100 μl to about 10 ml of saline solution containing a dose of about 1×10⁷ TCID₅₀ to 1×10⁹ TCID₅₀ (50% Tissue Culture Infective Dose) or Inf.U. (Infectious Unit). The rMVA may be administered in a volume ranging between 0.25 and 1.0 ml.

The recombinant GAd20 virus may be administered in an amount of about 10⁸ IFU per dose. In some embodiments, a composition comprising the GAd20 virus is administered at about 1×10¹⁰ IFU per dose. In some embodiments, a composition comprising the GAd20 virus is administered at about 1×10¹⁰ VP per dose. In some embodiments, a composition comprising the GAd20 virus is administered at about 1×10¹¹ VP per dose.

The compositions comprising self-replicating RNA molecule may be administered at a dose from about 1 microgram to about 100 microgram, about 1 microgram to about 90 micrograms, about 1 microgram to about 80 microgram, about 1 microgram to about 70 micrograms, about 1 microgram to about 60 micrograms, about 1 microgram to about 50 micrograms, about 1 microgram to about 40 micrograms, about 1 microgram to about 30 micrograms, about 1 microgram to about 20 micrograms, about 1 microgram to about 10 micrograms, or about 1 microgram to about 5 micrograms of the self-replicating RNA molecule.

Boosting compositions may be administered two or more times, weeks or months after administration of the priming composition, for example, about 1 or 2 weeks or 3 weeks, or 4 weeks, or 6 weeks, or 8 weeks, or 12 weeks, or 16 weeks, or 20 weeks, or 24 weeks, or 28 weeks, or 32 weeks or one to two years after administration of the priming composition. Additional boosting compositions may be administered 6 weeks to 5 years after the initial boosting inoculation, such as 6, 7, 8, 9, 10, 11, 12, 13, 14, 15, 16, 17, 18, 19, 20, 21, 22, 23, 24 or 25 weeks, or 7, 8, 9, 10, 11 or 12 months, or 2, 3, 4 or 5 years, after the initial boosting inoculation. Optionally, the further boosting can be repeated one or more times as needed.

### Combination Therapies

The polynucleotides, polypeptides, vectors, viruses, vaccines, and vaccine combinations may be used in combination with one or more additional cancer therapeutics for treating prostate.

The methods of enhancing an immune response against a prostate cancer in a subject in a subject in need thereof can comprise administering to the subject:

an immunologically effective amount of a first polynucleotide or transgene encoding PSMA for priming the immune response;

an immunologically effective amount of a second polynucleotide or transgene encoding STEAP1 for further priming the immune response;

an immunologically effective amount of a third polynucleotide or transgene encoding PSMA and STEAP1 for boosting the immune response; and

one or more additional cancer therapeutics.

The methods of treating a subject afflicted with a prostate cancer can comprise administering to the subject:

an immunologically effective amount of a first polynucleotide or transgene encoding PSMA for priming the immune response;

an immunologically effective amount of a second polynucleotide or transgene encoding STEAP1 for further priming the immune response;

an immunologically effective amount of a third polynucleotide or transgene encoding PSMA and STEAP1 for boosting the immune response; and

one or more additional cancer therapeutics.

The additional cancer therapeutic agent may be a surgery, a chemotherapy, an androgen deprivation therapy, radiation therapy, targeted therapy or a checkpoint inhibitor, or any combination thereof. In some embodiments, the one or more additional cancer therapeutics is surgery. In some embodiments, the one or more additional cancer therapeutics is an androgen deprivation therapy. In some embodiments, the one or more additional cancer therapeutics is a radiation therapy. In some embodiments, the one or more additional cancer therapeutics is a targeted therapy. In some embodiments, the one or more additional cancer therapeutics is a checkpoint inhibitor.

In some embodiments, the one or more additional cancer therapeutics is an anti-CTLA-4 antibody. An exemplary anti-CTLA-4 antibody is ipilimumab (YERVOY®).

In some embodiments, the one or more additional cancer therapeutics is an anti-PD-1 antibody. Exemplary anti-PD-1 antibodies are nivolumab (OPDIVO®), pembrolizumab (KEYTRUDA®), sintilimab, cemiplimab (LIBTAYO®), tripolibamab, tislelizumab, spartalizumab, camrelizumab, dostralimab, genolimzumab or cetrelimab. The approved anti-PD-1 antibodies may be purchased via authorized distributor or pharmacy. The amino acid sequences of the anti-PD-1 antibodies in general can be found from USAN and/or INN submissions by the companies.

In some embodiments, the one or more additional cancer therapeutics is an anti-PD-L1 antibody. Exemplary anti-PD-L1 antibodies are envafolimab, atezolizumab (TECENTRIQ®), durvalumab (IMFINZI®) and avelumab (BAVENCIO®).

Any antibodies, such as anti-CTLA-4 antibodies may be generated in native or transgenic mice, rats, chicken, rabbits, llama using known methods and assaying the obtained antibodies for their ability to bind CTLA-4 and reverse the suppression of T cells mediated by CTLA-4 using as readouts for example IL-2 production or proliferation.

Exemplary chemotherapeutic agents are alkylating agents; nitrosoureas; antimetabolites; antitumor antibiotics; plant alkyloids; taxanes; hormonal agents; and miscellaneous agents, such as busulfan, carboplatin, chlorambucil, cisplatin, cyclophosphamide, dacarbazine, ifosfamide, mechlorethamine hydrochloride, melphalan, procarbazine, thiotepa, uracil mustard, 5-fluorouracil, 6-mercaptopurine, capecitabine, cytosine arabinoside, floxuridine, fludarabine, gemcitabine, methotrexate, thioguanine, dactinomycin, daunorubicin, doxorubicin, idarubicin, mitomycin-C, and mitoxantrone, vinblastine, vincristine, vindesine, vinorelbine, paclitaxel, docetaxel.

Exemplary androgen deprivation therapies include abiraterone acetate, ketoconazole, enzalutamide, galeterone, ARN-509 and orteronel (TAK-700) and surgical removal of the testicles.

Radiation therapy may be administered using various methods, including external-beam therapy, internal radiation therapy, implant radiation, stereotactic radiosurgery, systemic radiation therapy, radiotherapy and permanent or temporary interstitial brachytherapy. External-beam therapy involves three-dimensional, conformal radiation therapy where the field of radiation is designed, local radiation (e.g., radiation directed to a preselected target or organ), or focused radiation. Focused radiation may be selected from stereotactic radiosurgery, fractionated stereotactic radiosurgery or intensity-modulated radiation therapy. Focused radiation may have particle beam (proton), cobalt-60 (photon) linear accelerator (x-ray) as a radiation source (see e.g. WO 2012/177624). “Brachytherapy,” refers to radiation therapy delivered by a spatially confined radioactive material inserted into the body at or near a tumor or other proliferative tissue disease site, and includes exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner include both solids and liquids. The radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material may also be a fluid made from any solution of radionuclide(s), e.g., a solution of I-125 or I-131, or a radioactive fluid can be produced using a slurry of a suitable fluid containing small particles of solid radionuclides, such as Au-198, Y-90. The radionuclide(s) may be embodied in a gel or radioactive micro spheres.

Targeted therapies include anti-androgen therapies, inhibitors of angiogenesis such as bevacizumab, anti-PSA or anti-PSMA antibodies or vaccines enhancing immune responses to PSA or PSMA.

Exemplary checkpoint inhibitors are antagonists of PD-1, PD-L1, PD-L2, VISTA, BTNL2, B7-H3, B7-H4, HVEM, HHLA2, CTLA-4, LAG-3, TIM-3, BTLA, CD160, CEACAM-1, LAIR1, TGFβ, IL-10, Siglec family protein, KIR, CD96, TIGIT, NKG2A, CD112, CD47, SIRPA or CD244. “Antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. Antagonist may be an antibody, a soluble ligand, a small molecule, a DNA or RNA such as siRNA. Exemplary antagonists of checkpoint inhibitors are described in U.S. Pat. Publ. No. 2017/0121409.

### EXAMPLES

The following examples are provided to further describe some of the embodiments disclosed herein. The examples are intended to illustrate, not to limit, the disclosed embodiments.

### Example 1 Construction of Recombinant MVA.hPSMA.hSTEAP1

MVA.hPSMA.hSTEAP1 transgene was designed to encode the two human proteins PSMA and STEAP1, separated by the Self-Cleaving 2A Peptide. A T-cell enhancer (TcE) sequence was fused at N-Terminal of each of the two proteins. The transgene nucleotide sequence was designed as follows: the human PSMA and STEAP1 coding sequences (from genBank NM_004476.2 and NM_012449.2 respectively) were used but some nucleotide was changed to remove 5TnT motifs that may act as transcription termination signals in MVA (where “n” stands for any nucleotide), polyT stretches and regions with too high or too low GC content that may have hampered transgene synthesis. To avoid the insertion of repeated sequences in the transgene, two versions of the TcE sequence were used, encoding the same protein sequence but with different codon usage (TcE-v1 and TcE-v2). TcE-v1 and TcE-v2 (no ATG) were fused N-Term to PSMA and STEAP1 sequences respectively. The stop codon was removed from PSMA and ATG was removed from STEAP1. The resulting sequences were separated by the 2A motif and terminated by two stop codons (TAGTAA; SEQ ID NO: 34).

The resulting sequence was synthetized and subcloned into a shuttle plasmid via BamH1-Asc1 restriction sites. The resulting plasmid was named H433 and subjected to control digestion with BamH1 and Asc1 restriction enzymes.

H433 plasmid contained:

hPSMA.hSTEAP1 transgene under control of the poxoviral promoter P7.5 flanked by two repeated regions named “Z”; eGFP transgene under the control of the synthetic Sp promoter; Flank-III regions homologous to the MVA sequences in the Deletion-III locus; Ampicillin Resistance. The transgene in H433 was sequenced by Sanger sequencing.

MVA.hPSMA.hSTEAP1 was generated by two homologous recombination steps occurring in vivo in CEF cells. In the first recombination step, fresh CEF cells were infected with parental MVA-RED 476 MG and transfected with the H433 plasmid. MVA-RED 476 MG carry an expression cassette for the HcRed1-1 red fluorescent protein at the Deletion III locus. H433 plasmid had two expression cassettes (PSMA.STEAP1 transgene under the poxoviral P7.5 promoter and eGFP transgene under the synthetic Sp promoter) flanked by sequences homologous to the Deletion III locus. The eGFP transgene cassette was also flanked by a short repeated sequence (215 bp) indicated as “Z”. Homologous recombination occurred between flank-III regions, and resulted in generation of an intermediate recombinant MVA that carried both the transgene and the eGFP cassette (MVA-Green-PSMA.STEAP1). The day after the infected/transfected cells showed both Red and Green fluorescence, indicating the presence of both MVA-RED 476 MG and MVA-Green-PSMA.STEAP1 vectors. Cells were collected and lysed. To isolate MVA-Green-PSMA.STEAP1, fresh CEF cells were infected with the resulting lysate and those only infected by MVA-Green-PSMA.STEAP1 were isolated by FACS sorting of green cells and used to re-infect fresh CEF cells in 96 wells. After 5 days infected wells were collected. The sample #B1, only showing green fluorescence, was subjected to PCR identity and purity controls and confirmed to be negative for MVA-RED contamination, positive for eGFP transgene, and positive for the PSMA.STEAP1 transgene.

The second recombination step that involves the repeated Z regions present in the MVA-Green-PSMA.STEAP1 genome is a spontaneous event occurring in cells infected with this vector. Thus to obtain the final MVA.PSMA.STEAP1, fresh CEF cells were infected with the lysate from clone #B1. FACS sorting of non-fluorescent cells in MW96 wells was performed to isolate cells infected with the final recombinant MVA.PSMA.STEAP1 vector.

After 4 days, infected wells showing cytopathic effect and no fluorescence were collected, lysed and subjected to PCR identity and purity controls. Two clones (#B1.B1 and #B1.C2) were selected and subjected to a limiting dilution step. Several clones from the limiting dilution were collected and subjected again to PCR identity and purity controls and the clone #B1.C2.16, resulting correct from all the PCR controls performed, was finally selected for the subsequent amplification steps.

P7.5 Promoter
SEQ ID NO: 1
GATCACTAATTCCAAACCCACCCGCTTTTTATAGTAAGTTTTTCACCCATAAATAATAAATACAA
TAATTAATTTCTCGTAAAAGTAGAAAATATATTCTAATTTATTGCACGGTAAGGAAGTAGAATCA
TAAAGAACAG
TrE-v1
SEQ ID NO: 2
ATGGGCCAGAAGGAACAGATTCATACGCTTCAGAAAAATTCTGAACGAATGTCAAAGCAATTGAC
ACGAAGTTCTCAGGCAGTA
hPSMA DNA
SEQ ID NO: 3
TGGAATCTCCTTCACGAAACCGACTCGGCTGTGGCTACCGCACGCAGACCTAGGTGGCTGTGTGC
TGGAGCTCTGGTGCTGGCGGGTGGCTTCTTTCTCCTCGGCTTCCTCTTCGGGTGGTTTATAAAAT
CCTCCAATGAAGCTACTAACATTACTCCAAAGCATAATATGAAAGCATTTTTGGATGAATTGAAA
GCTGAGAACATCAAGAAGTTCTTATATAATTTTACACAGATACCACATTTAGCAGGAACAGAACA
AAACTTTCAGCTTGCAAAGCAAATTCAATCCCAGTGGAAAGAATTTGGCCTGGATTCTGTTGAGC
TAGCACATTATGATGTCCTGTTGTCCTACCCAAATAAGACTCATCCCAACTACATCTCAATAATT
AATGAAGATGGAAATGAGATTTTCAACACATCATTATTTGAACCACCTCCTCCAGGATATGAAAA
TGTTTCGGATATTGTACCACCTTTCAGTGCTTTCTCTCCTCAAGGAATGCCAGAGGGCGATCTAG
TGTATGTTAACTATGCACGAACTGAAGACTTCTTTAAATTGGAACGGGACATGAAAATCAATTGC
TCTGGGAAAATTGTAATTGCCAGATATGGGAAAGTTTTCAGAGGAAATAAGGTTAAAAATGCCCA
GCTGGCAGGGGCCAAAGGAGTCATTCTCTACTCCGACCCTGCTGACTACTTTGCTCCTGGGGTGA
AGTCCTATCCAGATGGTTGGAATCTTCCTGGAGGTGGTGTCCAGCGTGGAAATATCCTAAATCTG
AATGGTGCAGGAGACCCTCTCACACCAGGTTACCCAGCAAATGAATATGCTTATAGGCGTGGAAT
TGCAGAGGCTGTTGGTCTTCCAAGTATTCCTGTTCATCCAATTGGATACTATGATGCACAGAAGC
TCCTAGAAAAAATGGGTGGCTCAGCACCACCAGATAGCAGCTGGAGAGGAAGTCTCAAAGTGCCC
TACAATGTTGGACCTGGCTTTACTGGAAACTTTTCTACACAAAAAGTCAAGATGCACATCCACTC
TACCAATGAAGTGACAAGAATTTACAATGTGATAGGTACTCTCAGAGGAGCAGTGGAACCAGACA
GATATGTCATTCTGGGAGGTCACCGGGACTCATGGGTGTTTGGTGGTATTGACCCTCAGAGTGGA
GCAGCTGTTGTTCATGAAATTGTGAGGAGCTTTGGAACACTGAAAAAGGAAGGGTGGAGACCTAG
AAGAACAATTTTGTTTGCAAGCTGGGATGCAGAAGAATTTGGTCTTCTTGGTTCTACTGAGTGGG
CAGAGGAGAATTCAAGACTCCTTCAAGAGCGTGGCGTGGCTTATATTAATGCTGACTCATCTATA
GAAGGAAACTACACTCTGAGAGTTGATTGTACACCGCTGATGTACAGCTTGGTACACAACCTAAC
AAAAGAGCTGAAAAGCCCTGATGAAGGCTTTGAAGGCAAATCTCTTTATGAAAGTTGGACTAAAA
AAAGTCCTTCCCCAGAGTTCAGTGGCATGCCCAGGATAAGCAAATTGGGATCTGGAAATGATTTT
GAGGTGTTCTTCCAACGACTTGGAATTGCTTCAGGCAGAGCACGGTATACTAAAAATTGGGAAAC
AAACAAATTCAGCGGCTATCCACTGTATCACAGTGTCTATGAAACATATGAGTTGGTGGAAAAGT
TTTATGATCCAATGTTTAAATATCACCTCACTGTGGCCCAGGTTCGAGGAGGGATGGTGTTTGAG
CTAGCCAATTCCATAGTGCTCCCTTTTGATTGTCGAGATTATGCTGTAGTTTTAAGAAAGTATGC
TGACAAAATCTACAGTATTTCTATGAAACATCCACAGGAAATGAAGACATACAGTGTATCATTTG
ATTCACTCTTCTCTGCAGTAAAGAATTTTACAGAAATTGCTTCCAAGTTCAGTGAGAGACTCCAG
GACTTTGACAAAAGCAACCCAATAGTATTAAGAATGATGAATGATCAACTCATGTTTCTGGAAAG
AGCATTTATTGATCCATTAGGGTTACCAGACAGGCCATTCTATAGGCATGTCATCTATGCTCCAA
GCAGCCACAACAAGTATGCAGGGGAGTCATTCCCAGGAATTTATGATGCTCTGTTTGATATTGAA
AGCAAAGTGGACCCTTCCAAGGCCTGGGGAGAAGTGAAGAGACAGATTTATGTTGCAGCCTTCAC
AGTGCAGGCAGCTGCAGAGACTTTGAGTGAAGTAGCC
2A
SEQ ID NO: 4
GCGCCAGTAAAGCAGACATTAAACTTTGATTTGCTGAAACTTGCAGGTGATGTAGAGTCAAATCC
AGGTCCA
TcE-v2
SEQ ID NO: 5
GGACAGAAAGAGCAAATCCACACACTGCAGAAGAACAGCGAGAGGATGAGCAAACAGCTTACCAG
GTCATCCCAAGCTGTT
hSTEAP1 DNA
SEQ ID NO: 6
GAAAGCAGAAAAGACATCACAAACCAAGAAGAACTTTGGAAAATGAAGCCTAGGAGAAATTTAGA
AGAAGACGATTATTTGCATAAGGACACGGGAGAGACCAGCATGCTAAAAAGACCTGTGCTTTTGC
ATTTGCACCAAACAGCCCATGCTGATGAATTTGACTGCCCTTCAGAACTTCAGCACACACAGGAA
CTCTTTCCACAGTGGCACTTGCCAATTAAAATAGCTGCTATTATAGCATCTCTGACTTTTCTTTA
CACTCTTCTGAGGGAAGTAATTCACCCTTTAGCAACTTCCCATCAGCAATACTTCTATAAGATTC
CAATCCTGGTCATCAACAAAGTCTTGCCAATGGTTTCCATCACTCTCTTGGCATTGGTTTACCTG
CCAGGTGTGATAGCAGCAATTGTCCAACTTCATAATGGAACCAAGTATAAGAAGTTTCCACATTG
GTTGGATAAGTGGATGTTAACAAGAAAGCAGTTTGGGCTTCTCAGTTTCTTCTTTGCTGTACTGC
ATGCAATTTATAGTCTGTCTTACCCAATGAGGCGATCCTACAGATACAAGTTGCTAAACTGGGCA
TATCAACAGGTCCAACAAAATAAAGAAGATGCCTGGATTGAGCATGATGTTTGGAGAATGGAGAT
TTATGTGTCTCTGGGAATTGTGGGATTGGCAATACTGGCTCTGTTGGCTGTGACATCTATTCCAT
CTGTGAGTGACTCTTTGACATGGAGAGAATTTCACTATATTCAGAGCAAGCTAGGAATTGTTTCC
CTTCTACTGGGCACAATACACGCATTGATTTTTGCCTGGAATAAGTGGATAGATATAAAACAATT
TGTATGGTATACACCTCCAACTTTTATGATAGCTGTTTTCCTTCCAATTGTTGTCCTGATATTTA
AAAGCATACTATTCCTGCCATGCTTGAGGAAGAAGATACTGAAGATTAGACATGGTTGGGAAGAC
GTCACCAAAATTAACAAAACTGAGATATGTTCCCAGTTG
TcE-v1 protein
SEQ ID NO: 13
MGQKEQIHTLQKNSERMSKQLTRSSQAV
TcE-v2 protein
SEQ ID NO: 7
GQKEQIHTLQKNSERMSKQLTRSSQAV
hPSMA PROTEIN
SEQ ID NO: 8
WNLLHETDSAVATARRPRWLCAGALVLAGGFFLLGFLFGWFIKSSNEATNITPKHNMKAFLDELK
AENIKKFLYNFTQIPHLAGTEQNFQLAKQIQSQWKEFGLDSVELAHYDVLLSYPNKTHPNYISII
NEDGNEIFNTSLFEPPPPGYENVSDIVPPFSAFSPQGMPEGDLVYVNYARTEDFFKLERDMKINC
SGKIVIARYGKVFRGNKVKNAQLAGAKGVILYSDPADYFAPGVKSYPDGWNLPGGGVQRGNILNL
NGAGDPLTPGYPANEYAYRRGIAEAVGLPSIPVHPIGYYDAQKLLEKMGGSAPPDSSWRGSLKVP
YNVGPGFTGNFSTQKVKMHIHSTNEVTRIYNVIGTLRGAVEPDRYVILGGHRDSWVFGGIDPQSG
AAVVHEIVRSFGTLKKEGWRPRRTILFASWDAEEFGLLGSTEWAEENSRLLQERGVAYINADSSI
EGNYTLRVDCTPLMYSLVHNLTKELKSPDEGFEGKSLYESWTKKSPSPEFSGMPRISKLGSGNDF
EVFFQRLGIASGRARYTKNWETNKFSGYPLYHSVYETYELVEKFYDPMFKYHLTVAQVRGGMVFE
LANSIVLPFDCRDYAVVLRKYADKIYSISMKHPQEMKTYSVSFDSLFSAVKNFTEIASKFSERLQ
DFDKSNPIVLRMMNDQLMFLERAFIDPLGLPDRPFYRHVIYAPSSHNKYAGESFPGIYDALFDIE
SKVDPSKAWGEVKRQIYVAAFTVQAAAETLSEVA
2A PROTEIN (F2A)
SEQ ID NO: 9
APVKQTLNFDLLKLAGDVESNPGP
hSTEAP1 PROTEIN
SEQ ID NO: 10
ESRKDITNQEELWKMKPRRNLEEDDYLHKDTGETSMLKRPVLLHLHQTAHADEFDCPSELQHTQE
LFPQWHLPIKIAAIIASLTFLYTLLREVIHPLATSHQQYFYKIPILVINKVLPMVSITLLALVYL
PGVIAAIVQLHNGTKYKKFPHWLDKWMLTRKQFGLLSFFFAVLHAIYSLSYPMRRSYRYKLLNWA
YQQVQQNKEDAWIEHDVWRMEIYVSLGIVGLAILALLAVTSIPSVSDSLTWREFHYIQSKLGIVS
LLLGTTHALIFAWNKWIDIKQFVWYTPPTFMIAVFLPIVVLIFKSILFLPCLRKKILKIRHGWED
VTKINKTEICSQL
Full Insert (promoter excluded from the sequence)
SEQ ID NO: 11
ATGGGCCAGAAGGAACAGATTCATACGCTTCAGAAAAATTCTGAACGAATGTCAAAGCAATTGAC
ACGAAGTTCTCAGGCAGTATGGAATCTCCTTCACGAAACCGACTCGGCTGTGGCTACCGCACGCA
GACCTAGGTGGCTGTGTGCTGGAGCTCTGGTGCTGGCGGGTGGCTTCTTTCTCCTCGGCTTCCTC
TTCGGGTGGTTTATAAAATCCTCCAATGAAGCTACTAACATTACTCCAAAGCATAATATGAAAGC
ATTTTTGGATGAATTGAAAGCTGAGAACATCAAGAAGTTCTTATATAATTTTACACAGATACCAC
ATTTAGCAGGAACAGAACAAAACTTTCAGCTTGCAAAGCAAATTCAATCCCAGTGGAAAGAATTT
GGCCTGGATTCTGTTGAGCTAGCACATTATGATGTCCTGTTGTCCTACCCAAATAAGACTCATCC
CAACTACATCTCAATAATTAATGAAGATGGAAATGAGATTTTCAACACATCATTATTTGAACCAC
CTCCTCCAGGATATGAAAATGTTTCGGATATTGTACCACCTTTCAGTGCTTTCTCTCCTCAAGGA
ATGCCAGAGGGCGATCTAGTGTATGTTAACTATGCACGAACTGAAGACTTCTTTAAATTGGAACG
GGACATGAAAATCAATTGCTCTGGGAAAATTGTAATTGCCAGATATGGGAAAGTTTTCAGAGGAA
ATAAGGTTAAAAATGCCCAGCTGGCAGGGGCCAAAGGAGTCATTCTCTACTCCGACCCTGCTGAC
TACTTTGCTCCTGGGGTGAAGTCCTATCCAGATGGTTGGAATCTTCCTGGAGGTGGTGTCCAGCG
TGGAAATATCCTAAATCTGAATGGTGCAGGAGACCCTCTCACACCAGGTTACCCAGCAAATGAAT
ATGCTTATAGGCGTGGAATTGCAGAGGCTGTTGGTCTTCCAAGTATTCCTGTTCATCCAATTGGA
TACTATGATGCACAGAAGCTCCTAGAAAAAATGGGTGGCTCAGCACCACCAGATAGCAGCTGGAG
AGGAAGTCTCAAAGTGCCCTACAATGTTGGACCTGGCTTTACTGGAAACTTTTCTACACAAAAAG
TCAAGATGCACATCCACTCTACCAATGAAGTGACAAGAATTTACAATGTGATAGGTACTCTCAGA
GGAGCAGTGGAACCAGACAGATATGTCATTCTGGGAGGTCACCGGGACTCATGGGTGTTTGGTGG
TATTGACCCTCAGAGTGGAGCAGCTGTTGTTCATGAAATTGTGAGGAGCTTTGGAACACTGAAAA
AGGAAGGGTGGAGACCTAGAAGAACAATTTTGTTTGCAAGCTGGGATGCAGAAGAATTTGGTCTT
CTTGGTTCTACTGAGTGGGCAGAGGAGAATTCAAGACTCCTTCAAGAGCGTGGCGTGGCTTATAT
TAATGCTGACTCATCTATAGAAGGAAACTACACTCTGAGAGTTGATTGTACACCGCTGATGTACA
GCTTGGTACACAACCTAACAAAAGAGCTGAAAAGCCCTGATGAAGGCTTTGAAGGCAAATCTCTT
TATGAAAGTTGGACTAAAAAAAGTCCTTCCCCAGAGTTCAGTGGCATGCCCAGGATAAGCAAATT
GGGATCTGGAAATGATTTTGAGGTGTTCTTCCAACGACTTGGAATTGCTTCAGGCAGAGCACGGT
ATACTAAAAATTGGGAAACAAACAAATTCAGCGGCTATCCACTGTATCACAGTGTCTATGAAACA
TATGAGTTGGTGGAAAAGTTTTATGATCCAATGTTTAAATATCACCTCACTGTGGCCCAGGTTCG
AGGAGGGATGGTGTTTGAGCTAGCCAATTCCATAGTGCTCCCTTTTGATTGTCGAGATTATGCTG
TAGTTTTAAGAAAGTATGCTGACAAAATCTACAGTATTTCTATGAAACATCCACAGGAAATGAAG
ACATACAGTGTATCATTTGATTCACTCTTCTCTGCAGTAAAGAATTTTACAGAAATTGCTTCCAA
GTTCAGTGAGAGACTCCAGGACTTTGACAAAAGCAACCCAATAGTATTAAGAATGATGAATGATC
AACTCATGTTTCTGGAAAGAGCATTTATTGATCCATTAGGGTTACCAGACAGGCCATTCTATAGG
CATGTCATCTATGCTCCAAGCAGCCACAACAAGTATGCAGGGGAGTCATTCCCAGGAATTTATGA
TGCTCTGTTTGATATTGAAAGCAAAGTGGACCCTTCCAAGGCCTGGGGAGAAGTGAAGAGACAGA
TTTATGTTGCAGCCTTCACAGTGCAGGCAGCTGCAGAGACTTTGAGTGAAGTAGCCGcgccagta
aagcagacattaaactttgatttgctgaaacttgcaggtgatgtagagtcaaatccaggtccaGG
ACAGAAAGAGCAAATCCACACACTGCAGAAGAACAGCGAGAGGATGAGCAAACAGCTTACCAGGT
CATCCCAAGCTGTTGAAAGCAGAAAAGACATCACAAACCAAGAAGAACTTTGGAAAATGAAGCCT
AGGAGAAATTTAGAAGAAGACGATTATTTGCATAAGGACACGGGAGAGACCAGCATGCTAAAAAG
ACCTGTGCTTTTGCATTTGCACCAAACAGCCCATGCTGATGAATTTGACTGCCCTTCAGAACTTC
AGCACACACAGGAACTCTTTCCACAGTGGCACTTGCCAATTAAAATAGCTGCTATTATAGCATCT
CTGACTTTTCTTTACACTCTTCTGAGGGAAGTAATTCACCCTTTAGCAACTTCCCATCAGCAATA
CTTCTATAAGATTCCAATCCTGGTCATCAACAAAGTCTTGCCAATGGTTTCCATCACTCTCTTGG
CATTGGTTTACCTGCCAGGTGTGATAGCAGCAATTGTCCAACTTCATAATGGAACCAAGTATAAG
AAGTTTCCACATTGGTTGGATAAGTGGATGTTAACAAGAAAGCAGTTTGGGCTTCTCAGTTTCTT
CTTTGCTGTACTGCATGCAATTTATAGTCTGTCTTACCCAATGAGGCGATCCTACAGATACAAGT
TGCTAAACTGGGCATATCAACAGGTCCAACAAAATAAAGAAGATGCCTGGATTGAGCATGATGTT
TGGAGAATGGAGATTTATGTGTCTCTGGGAATTGTGGGATTGGCAATACTGGCTCTGTTGGCTGT
GACATCTATTCCATCTGTGAGTGACTCTTTGACATGGAGAGAATTTCACTATATTCAGAGCAAGC
TAGGAATTGTTTCCCTTCTACTGGGCACAATACACGCATTGATTTTTGCCTGGAATAAGTGGATA
GATATAAAACAATTTGTATGGTATACACCTCCAACTTTTATGATAGCTGTTTTCCTTCCAATTGT
TGTCCTGATATTTAAAAGCATACTATTCCTGCCATGCTTGAGGAAGAAGATACTGAAGATTAGAC
ATGGTTGGGAAGACGTCACCAAAATTAACAAAACTGAGATATGTTCCCAGTTGTAGTAAA
Full insert protein
SEQ ID NO: 12
(TcE-vl-PSMA-2A-TcE-v2-STEAP1)
MGQKEQIHTLQKNSERMSKQLTRSSQAVWNLLHETDSAVATARRPRWLCAGALVLAGGFFLLGFL
FGWFIKSSNEATNITPKHNMKAFLDELKAENIKKFLYNFTQIPHLAGTEQNFQLAKQIQSQWKEF
GLDSVELAHYDVLLSYPNKTHPNYISIINEDGNEIFNTSLFEPPPPGYENVSDIVPPFSAFSPQG
MPEGDLVYVNYARTEDFFKLERDMKINCSGKIVIARYGKVFRGNKVKNAQLAGAKGVILYSDPAD
YFAPGVKSYPDGWNLPGGGVQRGNILNLNGAGDPLTPGYPANEYAYRRGIAEAVGLPSIPVHPIG
YYDAQKLLEKMGGSAPPDSSWRGSLKVPYNVGPGFTGNFSTQKVKMHIHSTNEVTRIYNVIGTLR
GAVEPDRYVILGGHRDSWVFGGIDPQSGAAVVHEIVRSFGTLKKEGWRPRRTILFASWDAEEFGL
LGSTEWAEENSRLLQERGVAYINADSSIEGNYTLRVDCTPLMYSLVHNLTKELKSPDEGFEGKSL
YESWTKKSPSPEFSGMPRISKLGSGNDFEVFFQRLGIASGRARYTKNWETNKFSGYPLYHSVYET
YELVEKFYDPMFKYHLTVAQVRGGMVFELANSIVLPFDCRDYAVVLRKYADKIYSISMKHPQEMK
TYSVSFDSLFSAVKNFTEIASKFSERLQDFDKSNPIVLRMMNDQLMFLERAFIDPLGLPDRPFYR
HVIYAPSSHNKYAGESFPGIYDALFDIESKVDPSKAWGEVKRQIYVAAFTVQAAAETLSEVAAPV
KQTLNFDLLKLAGDVESNPGPGQKEQIHTLQKNSERMSKQLTRSSQAVESRKDITNQEELWKMKP
RRNLEEDDYLHKDTGETSMLKRPVLLHLHQTAHADEFDCPSELQHTQELFPQWHLPIKIAAIIAS
LTFLYTLLREVIHPLATSHQQYFYKIPILVINKVLPMVSITLLALVYLPGVIAAIVQLHNGTKYK
KFPHWLDKWMLTRKQFGLLSFFFAVLHAIYSLSYPMRRSYRYKLLNWAYQQVQQNKEDAWIEHDV
WRMEIYVSLGIVGLAILALLAVTSIPSVSDSLTWREFHYIQSKLGIVSLLLGTIHALIFAWNKWI
DIKQFVWYTPPTFMIAVFLPIVVLIFKSILFLPCLRKKILKIRHGWEDVTKINKTEICSQL

### Example 2 Construction of Recombinant Ad26.hPSMA and Ad26.hSTEAP1

DNA encoding hPSMA or hSTEAP1 under the control of a CMV.TetO promoter was individually inserted into a replication incompetent Ad26 vector with deleted E1 (ΔE1A/E1B). Portion of the E3 region was also removed from the vector (4E3) to create sufficient space in the viral genome for insertion of the transgene. TetO operon in the vector comprises a 54-bp sequence containing two 19-bp tetracyline operator (TetO) sequences. CMV.TetO is repressible by the tetracycline repressor (TetR) protein to inhibit transgene expression during viral propagation. The vector also contains a SV40 polyA site.

The polynucleotide sequence of hPSMA in Ad26.hPSMA (SEQ ID NO: 14) encodes for hPSMA of SEQ ID NO: 15

SEQ ID NO: 14
ATGTGGAACCTGCTGCACGAGACAGACAGCGCCGTGGCCACAGCCCGGCGGCCCAGgTGGCTGTG
CGCAGGCGCCCTGGTGCTGGCAGGAGGCTTCTTTCTGCTGGGCTTCCTGTTTGGCTGGTTTATCA
AGAGCAGCAACGAGGCCACCAATATCACACCTAAGCACAATATGAAGGCCTTCCTGGAcGAGCTG
AAGGCCGAGAATATCAAGAAGTTCCTGTACAACTTTACCCAGATCCCACACCTGGCCGGCACAGA
GCAGAACTTTCAGCTGGCCAAGCAGATCCAGAGCCAGTGGAAGGAGTTCGGCCTGGACTCCGTGG
AGCTGGCCCACTACGAcGTGCTGCTGTCTTATCCAAATAAGACCCACCCCAACTATATCAGCATC
ATCAACGAGGACGGCAAcGAGATTTTCAACACATCTCTGTTTGAGCCCCCTCCACCCGGCTACGA
GAAcGTGAGCGACATCGTGCCTCCATTCTCTGCCTTTAGCCCACAGGGAATGCCTGAGGGCGATC
TGGTGTACGTGAATTAcGCCAGGACCGAGGACTTCTTTAAGCTGGAGCGCGATATGAAGATCAAC
TGTAGCGGCAAGATCGTGATCGCCCGGTACGGCAAGGTGTTTAGAGGCAATAAGGTGAAGAACGC
ACAGCTGGCAGGAGCAAAGGGCGTGATCCTGTACAGCGACCCCGCCGATTATTTCGCCCCTGGCG
TGAAGTCCTATCCAGACGGCTGGAATCTGCCAGGAGGAGGAGTGCAGAGGGGAAACATCCTGAAC
CTGAAcGGAGCAGGCGATCCTCTGACCCCAGGCTACCCCGCCAACGAGTACGCCTATAGGAGGGG
AATCGCAGAGGCAGTGGGCCTGCCTTCCATCCCAGTGCACCCCATCGGCTACTAcGACGCCCAGA
AGCTGCTGGAGAAGATGGGAGGCTCTGCCCCACCTGATTCTAGCTGGAGAGGCAGCCTGAAGGTG
CCTTACAAcGTGGGCCCAGGCTTCACCGGCAACTTTTCCACACAGAAGGTGAAGATGCACATCCA
CTCTACCAAcGAGGTGACAAGGATCTATAACGTGATCGGCACCCTGAGGGGAGCAGTGGAGCCTG
ACAGATACGTGATCCTGGGAGGACACAGGGACAGCTGGGTGTTTGGAGGAATCGATCCACAGTCC
GGAGCCGCCGTGGTGCACGAGATCGTGCGGTCCTTCGGCACCCTGAAGAAGGAGGGgTGGCGGCC
CCGGAGAACAATCCTGTTTGCCTCTTGGGAcGCCGAGGAGTTCGGCCTGCTGGGCTCCACAGAGT
GGGCAGAGGAGAACAGCCGGCTGCTCCAGGAGAGGGGAGTGGCCTACATCAAcGCCGACTCCTCT
ATCGAGGGCAACTATACCCTGCGGGTGGATTGCACACCCCTGATGTACTCCCTGGTGCACAACCT
GACCAAGGAGCTGAAGTCTCCTGACGAGGGCTTCGAGGGCAAGTCTCTGTAcGAGAGCTGGACAA
AGAAGTCTCCAAGCCCCGAGTTTAGCGGCATGCCTCGGATCTCCAAGCTGGGCTCTGGCAAcGAT
TTCGAGGTGTTCTTTCAGAGACTGGGAATCGCATCCGGCAGGGCCCGCTACACCAAGAATTGGGA
GACAAACAAGTTCTCTGGCTACCCACTGTATCACAGCGTGTACGAGACATACGAGCTGGTGGAGA
AGTTCTACGACCCCATGTTTAAGTATCACCTGACAGTGGCACAGGTGAGGGGAGGAATGGTGTTT
GAGCTGGCCAATAGCATCGTGCTGCCATTCGACTGTCGGGATTAcGCCGTGGTGCTGAGAAAGTA
CGCCGACAAAATCTACTCCATCTCTATGAAGCACCCCCAGGAGATGAAGACCTACAGCGTGTCCT
TCGATTCCCTGTTTTCTGCCGTGAAGAACTTCACAGAGATCGCCAGCAAGTTTTCCGAGCGGCTC
CAGGACTTCGATAAGTCCAATCCCATCGTGCTGAGGATGATGAACGACCAGCTGATGTTCCTGGA
GCGCGCCTTTATCGACCCTCTGGGCCTGCCTGATCGGCCCTTCTACAGACACGTGATCTAcGCCC
CTAGCTCCCACAACAAGTACGCCGGCGAGTCTTTTCCAGGCATCTAcGACGCCCTGTTCGATATC
GAGAGCAAGGTGGACCCCTCCAAGGCCTGGGGAGAGGTGAAGAGACAAATCTACGTGGCAGCCTT
CACCGTGCAGGCTGCAGCCGAGACACTGTCCGAGGTGGCC
SEQ ID NO: 15
(Ad26.hPSMA)
MWNLLHETDSAVATARRPRWLCAGALVLAGGFFLLGFLFGWFIKSSNEATNITPKHNMKAFLDEL
KAENIKKFLYNFTQIPHLAGTEQNFQLAKQIQSQWKEFGLDSVELAHYDVLLSYPNKTHPNYISI
INEDGNEIFNTSLFEPPPPGYENVSDIVPPFSAFSPQGMPEGDLVYVNYARTEDFFKLERDMKIN
CSGKIVIARYGKVFRGNKVKNAQLAGAKGVILYSDPADYFAPGVKSYPDGWNLPGGGVQRGNILN
LNGAGDPLTPGYPANEYAYRRGIAEAVGLPSIPVHPIGYYDAQKLLEKMGGSAPPDSSWRGSLKV
PYNVGPGFTGNFSTQKVKMHIHSTNEVTRIYNVIGTLRGAVEPDRYVILGGHRDSWVFGGIDPQS
GAAVVHEIVRSFGTLKKEGWRPRRTILFASWDAEEFGLLGSTEWAEENSRLLQERGVAYINADSS
IEGNYTLRVDCTPLMYSLVHNLTKELKSPDEGFEGKSLYESWTKKSPSPEFSGMPRISKLGSGND
FEVFFQRLGIASGRARYTKNWETNKFSGYPLYHSVYETYELVEKFYDPMFKYHLTVAQVRGGMVF
ELANSIVLPFDCRDYAVVLRKYADKIYSISMKHPQEMKTYSVSFDSLFSAVKNFTEIASKFSERL
QDFDKSNPIVLRMMNDQLMFLERAFIDPLGLPDRPFYRHVIYAPSSHNKYAGESFPGIYDALFDI
ESKVDPSKAWGEVKRQIYVAAFTVQAAAETLSEVA

The polynucleotide sequence of the transgene expression cassette in Ad26.hPSMA comprises a polynucleotide of SEQ ID NO: 16 encoding a polypeptide of SEQ ID NO: 14.

SEQ ID NO: 16
(full transgene expression cassette Ad26.hPSMA)
TCAATATTGGCCATTAGCCATATTATTCATTGGTTATATAGCATAAATCAATATTGGCTATTGGC
CATTGCATACGTTGTATCCATATCATAATATGTACATTTATATTGGCTCATGTCCAACATTACCG
CCATGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATAT
GCCAAGTACGCCCCCTATTGACGTCAATGACGGTAAATGGCCCGCCTGGCATTATGCCCAGTACA
TGACCTTATGGGACTTTCCTACTTGGCAGTACATCTACGTATTAGTCATCGCTATTACCATGGTG
ATGCGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGC
AGAGCTCTCCCTATCAGTGATAGAGATCTCCCTATCAGTGATAGAGATCGTCGACGAGCTCGTTT
AGTGAACCGTCAGATCGCCTGGAGACGCCATCCACGCTGTTTTGACCTCCATAGAAGACACCGGG
ACCGATCCAGCCTCCGCGGCCGGGAACGGTGCATTGGATCTAGAGCCACCATGTGGAACCTGCTG
CACGAGACAGACAGCGCCGTGGCCACAGCCCGGCGGCCCAGgTGGCTGTGCGCAGGCGCCCTGGT
GCTGGCAGGAGGCTTCTTTCTGCTGGGCTTCCTGTTTGGCTGGTTTATCAAGAGCAGCAACGAGG
CCACCAATATCACACCTAAGCACAATATGAAGGCCTTCCTGGAcGAGCTGAAGGCCGAGAATATC
AAGAAGTTCCTGTACAACTTTACCCAGATCCCACACCTGGCCGGCACAGAGCAGAACTTTCAGCT
GGCCAAGCAGATCCAGAGCCAGTGGAAGGAGTTCGGCCTGGACTCCGTGGAGCTGGCCCACTACG
AcGTGCTGCTGTCTTATCCAAATAAGACCCACCCCAACTATATCAGCATCATCAACGAGGACGGC
AAcGAGATTTTCAACACATCTCTGTTTGAGCCCCCTCCACCCGGCTACGAGAAcGTGAGCGACAT
CGTGCCTCCATTCTCTGCCTTTAGCCCACAGGGAATGCCTGAGGGCGATCTGGTGTACGTGAATT
AcGCCAGGACCGAGGACTTCTTTAAGCTGGAGCGCGATATGAAGATCAACTGTAGCGGCAAGATC
GTGATCGCCCGGTACGGCAAGGTGTTTAGAGGCAATAAGGTGAAGAACGCACAGCTGGCAGGAGC
AAAGGGCGTGATCCTGTACAGCGACCCCGCCGATTATTTCGCCCCTGGCGTGAAGTCCTATCCAG
ACGGCTGGAATCTGCCAGGAGGAGGAGTGCAGAGGGGAAACATCCTGAACCTGAAcGGAGCAGGC
GATCCTCTGACCCCAGGCTACCCCGCCAACGAGTACGCCTATAGGAGGGGAATCGCAGAGGCAGT
GGGCCTGCCTTCCATCCCAGTGCACCCCATCGGCTACTAcGACGCCCAGAAGCTGCTGGAGAAGA
TGGGAGGCTCTGCCCCACCTGATTCTAGCTGGAGAGGCAGCCTGAAGGTGCCTTACAAcGTGGGC
CCAGGCTTCACCGGCAACTTTTCCACACAGAAGGTGAAGATGCACATCCACTCTACCAAcGAGGT
GACAAGGATCTATAACGTGATCGGCACCCTGAGGGGAGCAGTGGAGCCTGACAGATACGTGATCC
TGGGAGGACACAGGGACAGCTGGGTGTTTGGAGGAATCGATCCACAGTCCGGAGCCGCCGTGGTG
CACGAGATCGTGCGGTCCTTCGGCACCCTGAAGAAGGAGGGgTGGCGGCCCCGGAGAACAATCCT
GTTTGCCTCTTGGGAcGCCGAGGAGTTCGGCCTGCTGGGCTCCACAGAGTGGGCAGAGGAGAACA
GCCGGCTGCTCCAGGAGAGGGGAGTGGCCTACATCAAcGCCGACTCCTCTATCGAGGGCAACTAT
ACCCTGCGGGTGGATTGCACACCCCTGATGTACTCCCTGGTGCACAACCTGACCAAGGAGCTGAA
GTCTCCTGACGAGGGCTTCGAGGGCAAGTCTCTGTAcGAGAGCTGGACAAAGAAGTCTCCAAGCC
CCGAGTTTAGCGGCATGCCTCGGATCTCCAAGCTGGGCTCTGGCAAcGATTTCGAGGTGTTCTTT
CAGAGACTGGGAATCGCATCCGGCAGGGCCCGCTACACCAAGAATTGGGAGACAAACAAGTTCTC
TGGCTACCCACTGTATCACAGCGTGTACGAGACATACGAGCTGGTGGAGAAGTTCTACGACCCCA
TGTTTAAGTATCACCTGACAGTGGCACAGGTGAGGGGAGGAATGGTGTTTGAGCTGGCCAATAGC
ATCGTGCTGCCATTCGACTGTCGGGATTAcGCCGTGGTGCTGAGAAAGTACGCCGACAAAATCTA
CTCCATCTCTATGAAGCACCCCCAGGAGATGAAGACCTACAGCGTGTCCTTCGATTCCCTGTTTT
CTGCCGTGAAGAACTTCACAGAGATCGCCAGCAAGTTTTCCGAGCGGCTCCAGGACTTCGATAAG
TCCAATCCCATCGTGCTGAGGATGATGAACGACCAGCTGATGTTCCTGGAGCGCGCCTTTATCGA
CCCTCTGGGCCTGCCTGATCGGCCCTTCTACAGACACGTGATCTAcGCCCCTAGCTCCCACAACA
AGTACGCCGGCGAGTCTTTTCCAGGCATCTAcGACGCCCTGTTCGATATCGAGAGCAAGGTGGAC
CCCTCCAAGGCCTGGGGAGAGGTGAAGAGACAAATCTACGTGGCAGCCTTCACCGTGCAGGCTGC
AGCCGAGACACTGTCCGAGGTGGCCtgaTAAGGTACCATCCGAACTTGTTTATTGCAGCTTATAA
TGGTTACAAATAAAGCAATAGCATCACAAATTTCACAAATAAAGCATTTTTTTCACTGCATTCTA
GTTGTGGTTTGTCCAAACTCATCAATGTATCTTATCATGTCT

The polynucleotide sequence of hSTEAP1 in Ad26.hSTEAP1 (SEQ ID NO: 17) encodes for hSTEAP1 of SEQ ID NO: 18

SEQ ID NO: 17
hSTEAP1 in Ad26.hSTEAP1
ATGGAGTCTCGGAAGGACATCACCAACCAGGAGGAGCTGTGGAAGATGAA
GCCACGGAGAAATCTGGAGGAGGACGATTACCTGCACAAGGATACCGGCG
AGACATCCATGCTGAAGCGGCCCGTGCTGCTGCACCTGCACCAGACCGCA
CACGCCGACGAGTTTGATTGCCCCTCTGAGCTGCAACACACACAGGAGCT
GTTCCCACAGTGGCACCTGCCCATCAAGATCGCCGCCATCATCGCCAGCC
TGACCTTTCTGTATACACTGCTGAGAGAAGTGATCCACCCTCTGGCCACC
TCCCACCAGCAGTACTTCTATAAGATCCCTATCCTGGTCATCAACAAGGT
GCTGCCAATGGTGAGCATCACACTGCTGGCCCTGGTGTACCTGCCTGGCG
TGATCGCCGCCATCGTGCAGCTGCACAAcGGCACCAAGTATAAGAAGTTT
CCACACTGGCTGGACAAGTGGATGCTGACACGCAAGCAGTTCGGCCTGCT
GTCTTTCTTTTTCGCCGTGCTGCACGCCATCTACAGCCTGTCCTATCCCA
TGAGGCGCAGCTACAGGTATAAGCTGCTGAACTGGGCCTACCAGCAGGTG
CAGCAGAATAAGGAGGACGCCTGGATCGAGCACGACGTGTGGCGCATGGA
AATCTACGTGAGCCTGGGAATCGTGGGCCTGGCAATCCTGGCCCTGCTGG
CAGTGACCTCTATCCCTTCTGTGAGCGACTCCCTGACcTGGCGGGAGTTT
CACTACATCCAGTCTAAGCTGGGCATCGTGAGCCTGCTGCTGGGCACCAT
CCACGCCCTGATCTTTGCCTGGAACAAGTGGATCGATATCAAGCAGTTCG
TGTGGTATACCCCCCCCACCTTCATGATCGCCGTGTTCCTGCCCATCGTG
GTGCTGATCTTTAAGAGCATCCTGTTCCTGCCTTGCCTGCGGAAGAAGAT
CCTGAAGATCAGACACGGCTGGGAGGAcGTGACCAAGATCAATAAGACAG
AGATTTGCAGCCAATTG
SEQ ID NO: 18
(initiator Met present)
MESRKDITNQEELWKMKPRRNLEEDDYLHKDTGETSMLKRPVLLHLHQTA
HADEFDCPSELQHTQELFPQWHLPIKIAAIIASLTFLYTLLREVIHPLAT
SHQQYFYKIPILVINKVLPMVSITLLALVYLPGVIAAIVQLHNGTKYKKF
PHWLDKWMLTRKQFGLLSFFFAVLHAIYSLSYPMRRSYRYKLLNWAYQQV
QQNKEDAWIEHDVWRMEIYVSLGIVGLAILALLAVTSIPSVSDSLTWREF
HYIQSKLGIVSLLLGTIHALIFAWNKWIDIKQFVWYTPPTFMIAVFLPIV
VLIFKSILFLPCLRKKILKIRHGWEDVTKINKTEICSQL

The polynucleotide sequence of the transgene expression cassette in Ad26.hSTEAP1 comprises a polynucleotide of SEQ ID NO: 19 encoding a polypeptide of SEQ ID NO: 18.

SEQ ID NO: 19
TCAATATTGGCCATTAGCCATATTATTCATTGGTTATATAGCATAAATCAATATTGGCTATTGGC
CATTGCATACGTTGTATCCATATCATAATATGTACATTTATATTGGCTCATGTCCAACATTACCG
CCATGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATAT
GCCAAGTACGCCCCCTATTGACGTCAATGACGGTAAATGGCCCGCCTGGCATTATGCCCAGTACA
TGACCTTATGGGACTTTCCTACTTGGCAGTACATCTACGTATTAGTCATCGCTATTACCATGGTG
ATGCGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGC
AGAGCTCTCCCTATCAGTGATAGAGATCTCCCTATCAGTGATAGAGATCGTCGACGAGCTCGTTT
AGTGAACCGTCAGATCGCCTGGAGACGCCATCCACGCTGTTTTGACCTCCATAGAAGACACCGGG
ACCGATCCAGCCTCCGCGGCCGGGAACGGTGCATTGGAGGATCCGCCACCATGGAGTCTCGGAAG
GACATCACCAACCAGGAGGAGCTGTGGAAGATGAAGCCACGGAGAAATCTGGAGGAGGACGATTA
CCTGCACAAGGATACCGGCGAGACATCCATGCTGAAGCGGCCCGTGCTGCTGCACCTGCACCAGA
CCGCACACGCCGACGAGTTTGATTGCCCCTCTGAGCTGCAACACACACAGGAGCTGTTCCCACAG
TGGCACCTGCCCATCAAGATCGCCGCCATCATCGCCAGCCTGACCTTTCTGTATACACTGCTGAG
AGAAGTGATCCACCCTCTGGCCACCTCCCACCAGCAGTACTTCTATAAGATCCCTATCCTGGTCA
TCAACAAGGTGCTGCCAATGGTGAGCATCACACTGCTGGCCCTGGTGTACCTGCCTGGCGTGATC
GCCGCCATCGTGCAGCTGCACAAcGGCACCAAGTATAAGAAGTTTCCACACTGGCTGGACAAGTG
GATGCTGACACGCAAGCAGTTCGGCCTGCTGTCTTTCTTTTTCGCCGTGCTGCACGCCATCTACA
GCCTGTCCTATCCCATGAGGCGCAGCTACAGGTATAAGCTGCTGAACTGGGCCTACCAGCAGGTG
CAGCAGAATAAGGAGGACGCCTGGATCGAGCACGACGTGTGGCGCATGGAAATCTACGTGAGCCT
GGGAATCGTGGGCCTGGCAATCCTGGCCCTGCTGGCAGTGACCTCTATCCCTTCTGTGAGCGACT
CCCTGACcTGGCGGGAGTTTCACTACATCCAGTCTAAGCTGGGCATCGTGAGCCTGCTGCTGGGC
ACCATCCACGCCCTGATCTTTGCCTGGAACAAGTGGATCGATATCAAGCAGTTCGTGTGGTATAC
CCCCCCCACCTTCATGATCGCCGTGTTCCTGCCCATCGTGGTGCTGATCTTTAAGAGCATCCTGT
TCCTGCCTTGCCTGCGGAAGAAGATCCTGAAGATCAGACACGGCTGGGAGGAcGTGACCAAGATC
AATAAGACAGAGATTTGCAGCCAATTGtgaTAACTCGAGATCCGAACTTGTTTATTGCAGCTTAT
AATGGTTACAAATAAAGCAATAGCATCACAAATTTCACAAATAAAGCATTTTTTTCACTGCATTC
TAGTTGTGGTTTGTCCAAACTCATCAATGTATCTTATCATGTCT
SEQ ID NO: 20
(CMV TetO promoter)
TCAATATTGGCCATTAGCCATATTATTCATTGGTTATATAGCATAAATCAATATTGGCTATTGGC
CATTGCATACGTTGTATCCATATCATAATATGTACATTTATATTGGCTCATGTCCAACATTACCG
CCATGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATCATAT
GCCAAGTACGCCCCCTATTGACGTCAATGACGGTAAATGGCCCGCCTGGCATTATGCCCAGTACA
TGACCTTATGGGACTTTCCTACTTGGCAGTACATCTACGTATTAGTCATCGCTATTACCATGGTG
ATGCGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGC
AGAGCTCTCCCTATCAGTGATAGAGATCTCCCTATCAGTGATAGAGATCGTCGACGAGCTCGTTT
AGTGAACCGTCAGATCGCCTGGAGACGCCATCCACGCTGTTTTGACCTCCATAGAAGACACCGGG
ACCGATCCAGCCTCCGCGGCCGGGAACGGTGCATTGGA
SEQ ID NO: 21
SV40pA
ATCCGAACTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTATCA
TGTCT

Ad26.PSMA and Ad26.STEAP1 vectors were generated on suspension PER.C6 TetR (sPER.C6 TetR) cells using standard operation procedures. PER.C6 TetR cells, which express TetR under control of an Aotine Herpesvirus 1 major immediate early promoter (AoHV-1 promoter), were generated by stable transfection of PER.C6 cells with plasmid pC_AoHV TetR as described in WO2018/146205. For preMVS production, PER.C6 TetR cells were transfected with the pAd26.PSMA or pAd26.STEAP1 single genome plasmid using Lipofectamine according to the instructions provided by the manufacturer (Life Technologies). Two plaque purification and amplification rounds were performed, in which plaques were isolated, selected and used to infect sPER.C6 TetR cells. After the 2^{nd} plaque purification and amplification round, the virus stocks were expanded for production. The virus was purified using a two-step cesium chloride centrifugation purification method. Finally, the virus was stored in aliquots at −85° C.

### Example 3 Ad26.hPSMA and Ad26.hSTEAP1 Induce T Cell Responses In Vitro Materials and Methods

Donor CD1c+ Dendritic Cells (DCs) were thawed, resuspended in complete media (IMDM containing 5% pooled human serum and 1% Pen/Strep; Invitrogen) plus GM-CSF (80 ng/mL; Peprotech) and IL-4 (80 ng/mL; Peprotech), and then seeded at 5×10⁴ cells per well. After resting overnight at 37° C., DCs were transduced with 75,000 virus particles of either Ad26.PSMA or Ad26.STEAP1 for a further 24 hours at 37° C. Cells were washed with media then 5×10⁵ autologous donor T cells were added to the DCs in media containing IL-2 (100 u/mL; R&D systems) and IL-15 (long/mL; Peprotech). A media exchange was performed every other day until day 13. On day 13, T cells were restimulated with 1 μg/mL of hSTEAP1 or hPSMA peptide pools (JPT Peptide Technologies) containing a protein transport inhibitor cocktail (ebioscience) for 16 hours. Cells were washed and stained for intracellular cytokine staining (ICS) analysis with a phenotyping and intracellular cytokine panel containing CD3, CD4, CD8, CD137, TNFα, IFNγ, and IL-2 (Biolegend). Stained cells were analyzed on an LSR Fortessa II (BD Biosciences).

### Results

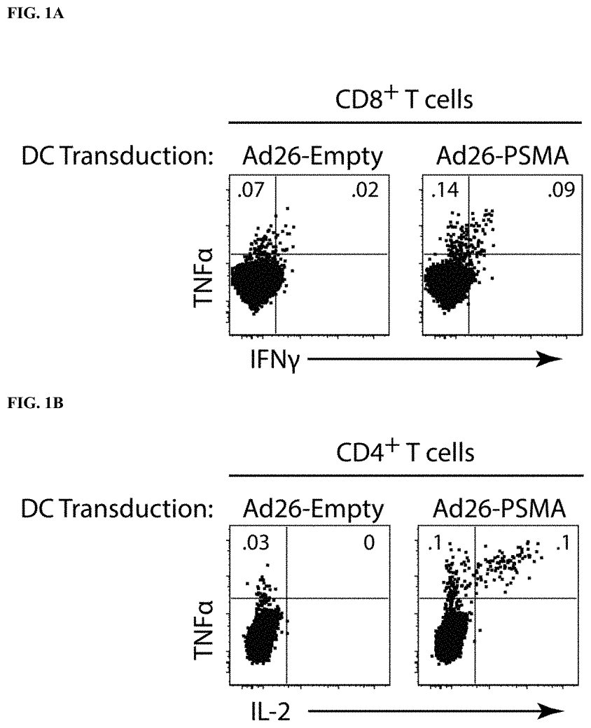

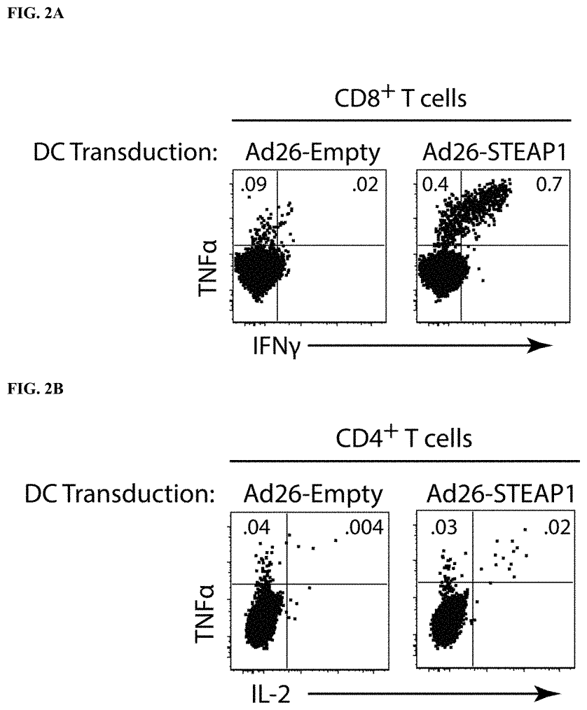

The ability of Ad26.hPSMA and Ad26.hSTEAP1 to induce T cell responses against their inserts (hPSMA and hSTEAP1) was assessed in vitro. Normal human donor CD1c+ dendritic cells were transduced with either Ad26.hPSMA or Ad26.hSTEAP1 and their ability to present vector-derived antigens to prime autologous donor CD8⁺ and CD4⁺ T cells was assessed. Transduced dendritic cells were combined with autologous T cells for 12 days then antigen-specific T cell responses were assayed using overlapping peptide pools of hPSMA or hSTEAP1. Antigen-specific T cell responses were evaluated by intracellular cytokine production of effector cytokines TNFα, IFNγ, and IL-2. Positive T cell recall responses were determined to be those with at least 0.05% of cells in a double positive cytokine-producing gate and at least a three-fold increase over averaged empty-vector control. Ten of the twelve screened normal male donors generated positive T cell responses to either hPSMA or hSTEAP1. FIG. 1 and FIG. 2 show a representative flow cytometry plots of ICS (TNFα, IFNγ, and IL-2) from a single donor showing antigen-specific CD8+ and CD4+ T cell recall responses 12 days following antigen priming by APCs transduced with either Ad26.hPSMA (FIG. 1) or Ad26.hSTEAP1 (FIG. 2). Numbers in the gates indicate the percentages of total CD8+ or CD4+ T cells staining positive for the respective cytokines. No statistical analyses were performed applicable to the displayed data. Table 1 shows the CD8⁺ and CD4⁺ T cell responses specific for hSTEAP1 or hPSMA for each of 12 normal male donors. Fold over averaged empty-vector control is indicated for positive responses. Importantly, these assays demonstrated that human donors contain precursor T cells that can be primed against hPSMA and hSTEAP1 by the Ad26.hPSMA and Ad26.hSTEAP1 vaccine vectors.

TABLE 1

| | STEAP1 | | PSMA | |
|---|---|---|---|---|
| Donor | CD8 | CD4 | CD8 | CD4 |
| 1 | 29.5x | 7.2x | 4.6x | 30.3x |
| 2 | 3.8x | 21.1x | — | 1886x |
| 3 | 3.2x | — | 4.7x | — |
| 4 | 694x | — | 24.4x | 24.5x |
| 5 | 5.7x | — | — | — |
| 6 | 42.4x | — | — | — |
| 7 | — | 23x | — | — |
| 8 | — | 8.8x | — | — |
| 9 | — | — | 17.9x | 22.3x |
| 10 | — | — | 154.6x | — |
| 11 | — | — | — | — |
| 12 | — | — | — | — |

“—” Indicates no positive response

### Example 4. Ad26.hPSMA and Ad26.hSTEAP1 Induce Immune Responses in Mice

### Materials and Methods

Immunization schedule. At day 0, groups of six mice were immunized with 10⁹ or 10¹⁰ vp of Ad26.hPSMA or Ad26.STEAP1. A control group of 10 mice received an Ad26 vector expressing no transgene (‘empty’). Two weeks after the immunization the animals were sacrificed and splenocytes were analyzed for induction of hPSMA or hSTEAP-1 specific cytokine-producing cells by IFNγ ELISpot or ICS. Splenocytes were stimulated overnight with hPSMA-specific or hSTEAP-1 specific peptide pools. For the ELISpot assay, the number of IFNγ spot forming units (SFU) per 10⁶ splenocytes was determined. The geometric mean response per group is indicated with a horizontal line. The dotted lines indicate the background of the assay defined as the 95% percentile of SFU observed in non-stimulated splenocytes. For statistical analysis using Wilcoxon Rank Sum test with Bonferroni correction, values below 18.5 SFU/10⁶ cells were set to this cut-off. Cytokine secreting cells were measured in CD4 or CD8⁺ gated CD3+ cells by ICS and FACS analysis; the dotted lines indicate the background of the assay defined as average response in non-stimulated splenocytes plus 3×standard deviations, and were calculated for each cytokine producing cell population, values below this value was set at this cut-off.

### Results

The ability of Ad26.hPSMA or Ad26.hSTEAP1 generated in Example 2 to induce cellular immune responses against vector-encoded antigen in C57BL/6 mice after intramuscular immunization was evaluated. Ad26.hPSMA and Ad26.hSTEAP1 were tested at two doses 10⁹ virus particles (vp) or 10¹⁰ vp, control mice received 10¹⁰ of an Ad26 vector not enclosing a transgene (Ad26-empty). Immune responses against the respective antigens were measured using known immunological assays, such as enzyme-linked immunospot assay (ELISPOT), or intracellular cytokine staining (ICS).

Animals were sacrificed two weeks post immunization and splenocytes were isolated. Different immune parameters were assessed as described below.

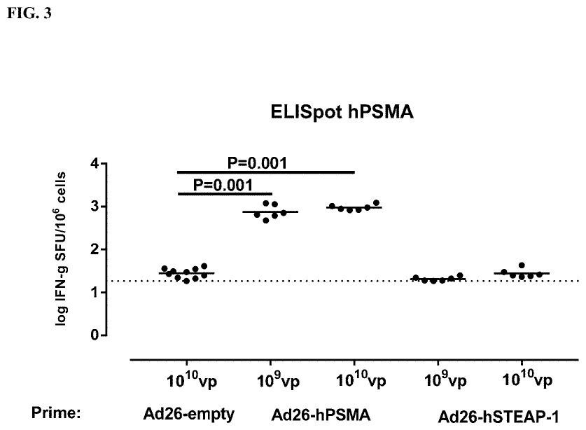

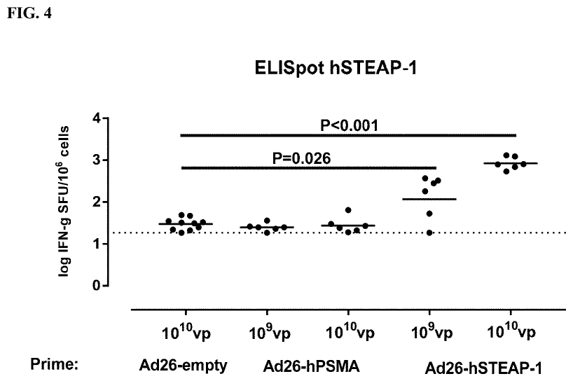

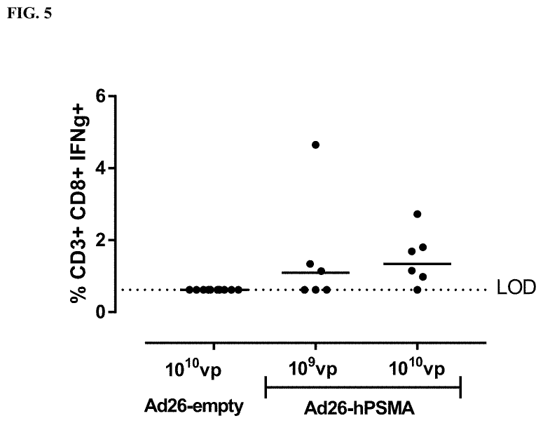

Cellular immune responses against the vector-encoded antigen was evaluated by hPSMA or hSTEAP-1 specific-IFNγ ELISPOT assay or ICS. To this end, splenocytes were stimulated overnight with a 15mer overlapping peptides spanning the hPSMA or hSTEAP1 wildtype antigen. The antigen specific immune responses were determined by measuring the relative number of IFNγ-secreting cells. The IFNγ ELISpot results showed that the cellular immune responses induced by Ad6.hPSMA were significantly higher than that induced by the Ad26-empty at both dosages tested. Similarly, the hSTEAP1 response was higher than that induced by the Ad26-empty vector at both dosages tested. A clear dose response was seen with Ad26.hSTEAP1 (FIG. 4), whereas there was minor difference in the magnitude of the response induced by 10⁹ vp or 10¹⁰ vp of Ad26.hPSMA (FIG. 4). As expected, no hPSMA or hSTEAP-1 specific responses were detected against the adenovectors lacking these antigens. The ICS results showed that mainly IFNγ producing CD8+ T-cells were induced, whereas the level of TNFα was overall low, there were no detectable induction of CD4+ specific cells producing cytokine. FIG. 3 shows the log of the number of IFNγ spot forming units (SFU) per 10⁶ splenocytes from splenocytes isolated from mice immunized with 10⁹ or 10¹⁰ virus particles (vp) Ad26.hPSMA1, Ad.26.STEAP1 or an empty vector (Ad26-empty) as indicated after stimulation overnight with hPSMA peptide pools. The geometric mean response per group is indicated with a horizontal line. The dotted lines indicate the background of the assay defined as the 95% percentile of SFU observed in non-stimulated splenocytes. IFNγ was measured using ELISpot. FIG. 4 shows the log of the number of IFNγ spot forming units (SFU) per 10⁶ splenocytes from splenocytes isolated from mice immunized with 10⁹ or 10¹⁰ virus particles (vp) Ad26.hPSMA1, Ad.26.STEAP1 or an empty vector (Ad26-empty) as indicated after stimulation overnight with hSTEAP1 peptide pools. The geometric mean response per group is indicated with a horizontal line. The dotted lines indicate the background of the assay defined as the 95% percentile of SFU observed in non-stimulated splenocytes. IFNγ was measured using ELISpot. FIG. 5 shows the percentage (%) of CD8+ spelenocytes producing IFNγ (CD3⁺CD8⁺IFNγ⁺ cells) isolated from mice immunized with 10⁹ or 10¹⁰ virus particles (vp) Ad26.hPSMA or an empty vector (Ad26-empty) as indicated after stimulation overnight with hPSMA peptide pools. The geometric mean response per group is indicated with a horizontal line. The dotted line shows the background of the assay defined as the mean plus 3× the standard deviation of the background staining, values below this value was set at this cut-off. IFNγ was measured using intracellular cytokine staining (ICS). FIG. 6 shows the percentage (%) of CD8+ spelenocytes producing IFNγ (CD3⁺CD8⁺IFNγ⁺ cells) isolated from mice immunized with 10⁹ or 10¹⁰ virus particles (vp) Ad26.hSTEAP1 or an empty vector (Ad26-empty) as indicated after stimulation overnight with hSTEAP1 peptide pools. The geometric mean response per group is indicated with a horizontal line. The dotted line shows the background of the assay defined as the mean plus 3× the standard deviation of the background staining, values below this value was set at this cut-off. IFNγ was measured using intracellular cytokine staining (ICS).

Overall, the cellular immune responses induced by Ad26.hPSMA and Ad26.hSTEAP1 clearly indicated potent immunogenicity of these constructs in mice.

### Example 5. Co-Injection of Ad26.hPSMA or Ad26.hSTEAP1 has Minor Impact on the Magnitude of the Cellular Immune Response Compared to Single Injected Vaccines

### Materials and Methods

At day 0, mice (n=9 per group) were immunized with 10⁸ or 10⁹ vp of Ad26.hPSMA alone or in combination with 10¹⁰ vp Ad26.hSTEAP1. Two weeks after the immunization the animals were sacrificed and splenocytes were analyzed for induction of hPSMA or hSTEAP1 specific cytokine producing cells by IFNγ ELISpot.

Splenocytes were stimulated overnight with hPSMA-specific or hSTEAP1-specific peptide pools. The number of IFNγ SFU per 10⁶ splenocytes was determined by ELISpot. The geometric mean response per group was measured. The background of the assay was defined as the 95% percentile of SFU observed in non-stimulated splenocytes. Values below 17.9 SFU/10⁶ cells were set to this cut-off. To test the difference between co-injection (co-ad) versus bedside mixing, an ANOVA analysis was conducted. Non-inferiority analysis was carried out comparing single injected vectors (10⁹ VP Ad26.hPSMA or 10¹⁰ Ad26.hSTEAP1) versus co-ad or bedside mixing, using a pre-specified margin of 0.5 log₁₀.

### Results

Ad26.hPSMA and Ad26.hSTEAP1 were co-injected into mice to evaluate their simultaneous impact on vaccine induced immunogenicity. In this experiment, the vectors were assessed for their ability to induce cellular immune responses against vector-encoded antigen in mice after intramuscular immunization. Ad26.hPSMA vector was tested at two doses 10⁸ vp or 10⁹ vp, whereas Ad26.hSTEAP1 was tested at 10¹⁰ vp. Immune responses against the respective antigens were measured using enzyme-linked immunospot assay (ELISPOT).

For the co-ad, mice received Ad26.hPSMA in one leg (10⁹ vp) and Ad26.hSTEAP1 in the other leg (10¹⁰ vp), and for the bedside mixing Ad26.hPSMA (10⁹ vp) and Ad26.hSTEAP1 (10¹⁰ vp) were mixed prior to injection and injected into one leg (bedside mixing). Animals were sacrificed two weeks post immunization and splenocytes were isolated.

Splenocytes were stimulated overnight with a 15mer overlapping peptides spanning the hPSMA or hSTEAP1 wild type antigen. The antigen specific immune responses were determined by measuring the relative number of IFNγ-secreting cells. The IFNγ ELISpot results showed that the hPSMA-specific cellular immune responses induced by injecting both Ad26.hPSMA and Ad26.hSTEAP1 either as co-administration or bedside mixing were non-inferior to the response induced by Ad26.PSMA only. Moreover, there was no significant difference in the magnitude of the response induced by co-administration or bed-side mixing. Similarly, for the hSTEAP1 response there were no significant difference in the magnitude of the immune response induced when comparing co-administration and bed-side mixing. The hSTEAP1 immune response induced by co-administration was non-inferior to that induced by the Ad26.hSTEAP1 alone, in contrast non-inferiority could not be shown for the bed-side mixing compared to the immune response induced by Ad26.hSTEAP1 alone.

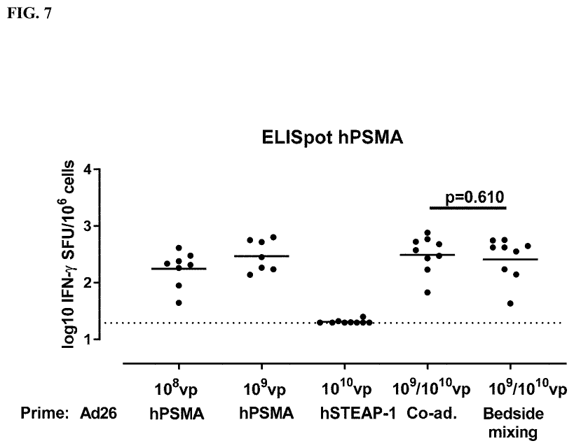

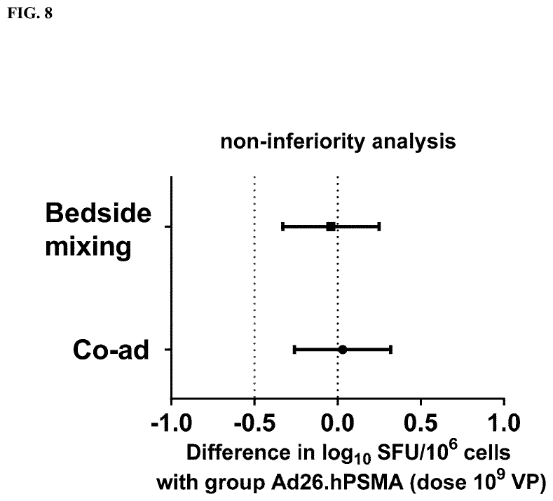

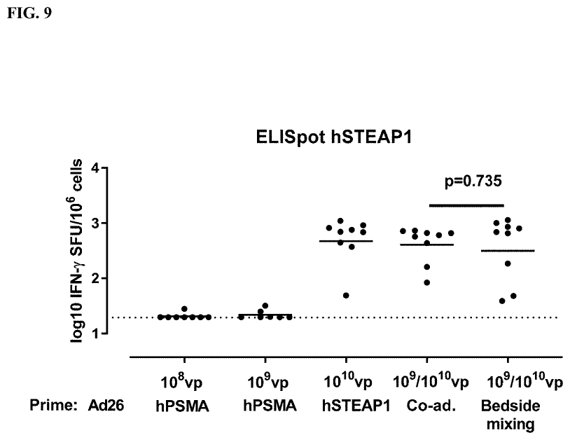

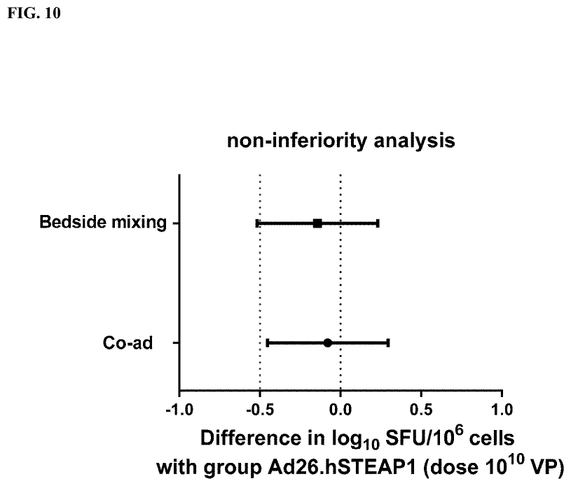

FIG. 7 shows the log of the number of IFNγ spot forming units (SFU) per 10⁶ splenocytes from splenocytes isolated from mice immunized with Ad26.hPSMA1, Ad.26.STEAP1, Ad26.hPSMA1 and Ad.26.STEAP1 each injected to separate legs, (co-ad) or Ad26.hPSMA1 and Ad.26.STEAP1 mixed prior to injection and injected into one leg (bedside mixing) as indicated after stimulation overnight with hPSMA peptide pools. The geometric mean response per group is indicated with a horizontal line. The dotted lines indicate the background of the assay defined as the 95% percentile of SFU observed in non-stimulated splenocytes. IFNγ was measured using ELISpot. No statistically significant difference was observed between the co-ad and bedside mixing groups. FIG. 8 shows the non-inferiority analyses demonstrating that the induced hPSMA-specific immune response by bedside mixing and co-ad was non-inferior to that induced by Ad26.hPSMA. FIG. 9 shows the log of the number of IFNγ spot forming units (SFU) per 10⁶ splenocytes from splenocytes isolated from mice immunized with Ad26.hPSMA1, Ad.26.STEAP1, Ad26.hPSMA1 and Ad.26.STEAP1 each injected to separate legs, (co-ad) or Ad26.hPSMA1 and Ad.26.STEAP1 mixed prior to injection and injected into one leg (bedside mixing) as indicated after stimulation overnight with hSTEAP1 peptide pools. The geometric mean response per group is indicated with a horizontal line. The dotted lines indicate the background of the assay defined as the 95% percentile of SFU observed in non-stimulated splenocytes. IFNγ was measured using ELISpot. No statistically significant difference was observed between the co-ad and bedside mixing groups. FIG. 10 shows the non-inferiority analyses demonstrating that hSTEAP1-specific immune response by bedside mixing was non-inferior to that induced by Ad26.hSTEAP1, whereas non-inferiority could not be shown for bedside mixing compared to Ad26.hSTEAP1.

Overall, the cellular immune responses induced by co-injection of Ad26.hPSMA and Ad26.hSTEAP-1 had minor impact on the overall magnitude of the response.

### Example 6. Ad26.hPSMA Delays Tumor Growth of CT26-PSMA Tumors In Vivo

### Materials and Methods

A murine CT26 colorectal cancer cell line expressing hPSMA (CT26-PSMA cells) was generated by transducing CT26 cells with lentivirus encoding the full length hPSMA sequence (GenBank: M99487.1) driven by an EF1a promoter (Genecopoeia). Transduced cells were single cell cloned and clonal populations were screened by flow cytometry using an antibody detecting hPSMA (Biolegend) compared to the CT26 parental cell line. A clonal population of cells expressing low levels of hPSMA was selected and growth kinetics were compared to the CT26 parental cell line by implanting 5×10⁵ cells in Balb/c mice and tracking tumor growth over time.

To test the efficacy of Ad26.hPSMA constructs at inducing effective T cell responses that could control tumor growth, mice were subcutaneously implanted with 5×10⁵ CT26-PSMA cells on the lateral flank then on day 14 post-implant treated with either 10¹⁰ vp Ad26-Empty vector, 10¹⁰ vp Ad26-Empty vector plus anti-CTLA-4 antibody (5 mg/kg), 10¹⁰ vp Ad26.hPSMA, and 10¹⁰ vp Ad26.hPSMA plus anti-CTLA-4 antibody (5 mg/kg) then tumor growth was measured over time. On day 10 post treatment, PBMCs were isolated and antigen-specific recall responses were evaluated by stimulating cells with overlapping peptide pool to hPSMA (JPT Peptide Technologies) for 5 hours and then evaluating IFNγ intracellular cytokine production by flow cytometry.

### Results

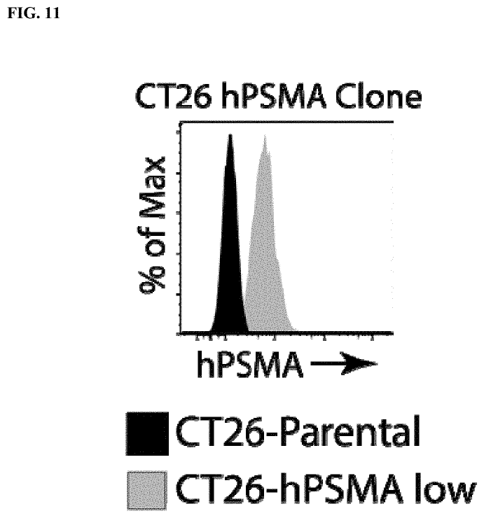

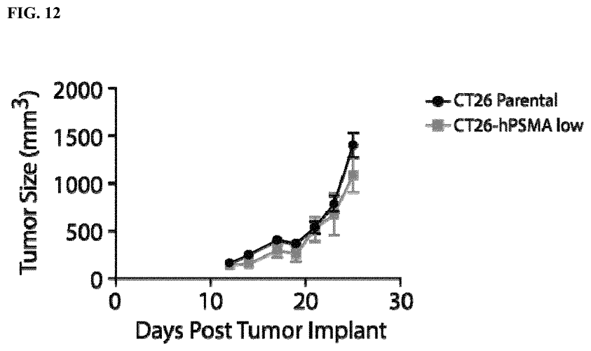

To assess the quality of immune response generated by Ad26.hPSMA, a syngeneic murine tumor model was designed to express the vaccine target PSMA. The CT26 syngeneic tumor model was used as it is an easily measured subcutaneous tumor commonly employed to study immune-oncology agents. CT26 cells were transduced with lentivirus encoding hPSMA and single cell clones were isolated to ensure uniform hPSMA expression. FIG. 11 shows flow cytometry measuring hPSMA expression on a selected clonally expanded CT26 tumor cell transduced with hPSMA. Tumor growth kinetics of CT26-PSMA cells in vivo were compared to parental CT26 cells to confirm that the addition of hPSMA did not alter tumor growth (FIG. 12).

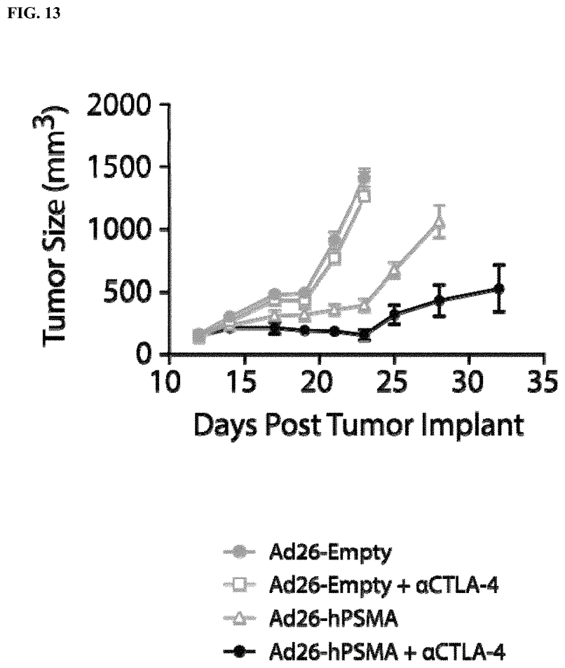

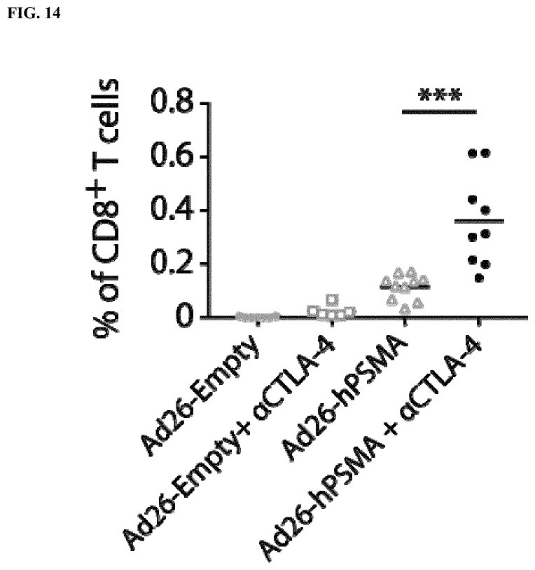

Using the CT26-PSMA tumor model, the ability of T cell responses generated by Ad26.hPSMA treatment to help control tumor growth was assessed as a single agent or in combination with immune checkpoint blockade anti-CTLA-4 antibody. Compared to treatment with an Ad26.Empty vector control or Ad26.Empty vector in combination with anti-CTLA-4 antibody, mice treated with Ad26.hPSMA showed delayed tumor growth, and the combination of Ad26.hPSMA and anti-CTLA-4 antibody further enhanced tumor control as shown in FIG. 13. Antigen-specific CD8⁺ T cell responses were also assessed in the blood of mice treated with each of the agents by peptide restimulation using an overlapping peptide pool to hPSMA. Restimulation responses showed hPSMA-specific T cells were successfully detected in mice treated with Ad26.hPSMA and the combination of Ad26.hPSMA and anti-CTLA-4 antibody showed the highest level of antigen-specific CD8⁺ T cells that was significantly higher than either single agent alone. FIG. 14 shows the percentage (%) of IFNγ⁺CD8⁺ T cells of total CD8⁺ T cells after indicated treatments.

### Example 7. Prime-Boost Regiments of Ad26.hPSMA, Ad26.hSTEAP1 and MVA.hPSMA.hSTEAP1 Enhance Immune Responses in Mice Over Prime with Ad26.hPSMA and Ad26.hSTEAP1

### Materials and Methods

Animals were immunized as follows: Group1 (n=5) were primed with MVA.hPSMA.hSTEAP1 (10⁷ IU) at week 3; Group 3 (n=10) and Group 4 (n=10) were prime immunized i.m. with a mixture of Ad26.hPSMA (10⁸ vp)+Ad26.hSTEAP-1 (10⁹ vp) on week 0, followed by a boost at week 3 with MVA.hPSMA.hSTEAP (10⁷ IU); group 10 served as the assay negative control group (n=3) was prime immunized i.m. with Ad26.Empty at 10¹⁰ vp on week 0. All animals were sacrificed in week 4 e.g. 7 days post the MVA immunization.

Splenocytes were analyzed for induction of hPSMA or hSTEAP1 specific cytokine producing cells by IFNγ ELISpot. The number of IFNγ SFU per 10⁶ splenocytes was determined by ELISpot. The geometric mean response per group was determined and the background of the assay defined as the 95% percentile of SFU observed in non-stimulated splenocytes. For difference testing comparing Ad26.hPSMA+Ad26.hSTEAP1 prime-only with Ad26.hPSMA+Ad26.hSTEAP1 prime immunization and boost with MVA.hPSMA.hSTEAP1, an ANOVA was performed on log₁₀-transformed ELISpot data. Values below 22 SFU/10⁶ cells were set as cut-off (corresponding to 1.342 log 10).

### Results

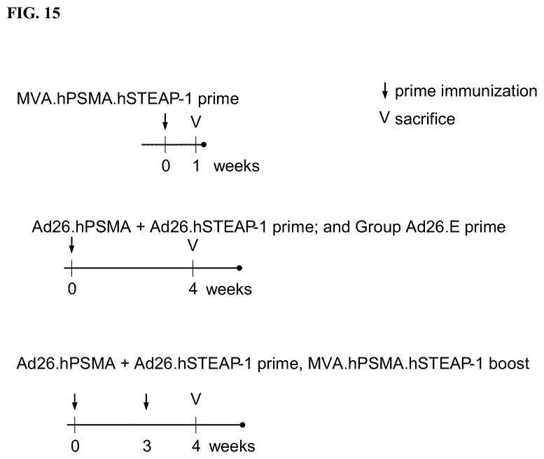

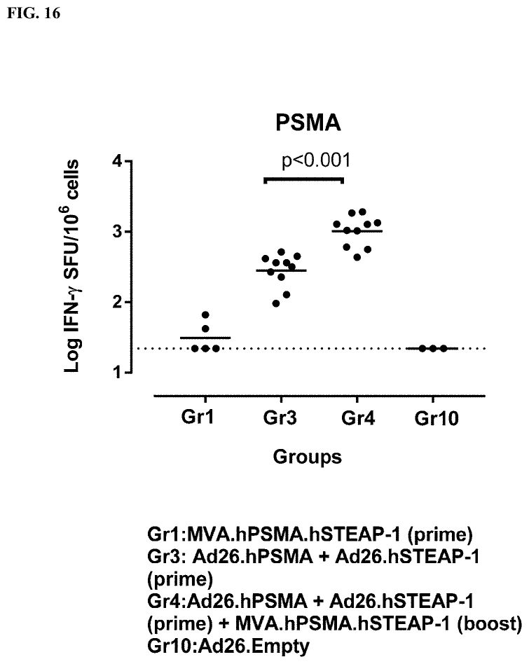

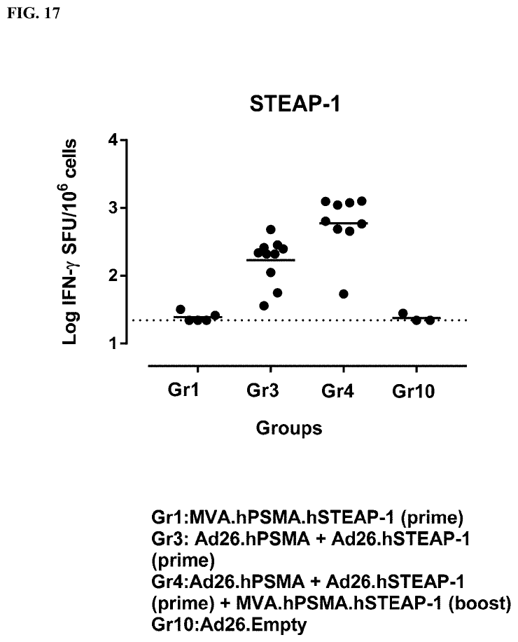

As a priming immunization, mice were vaccinated by intramuscular injection with Ad26.hPSMA (10⁸vp) and Ad26. hSTEAP1 (10⁹ vp), or as a control with adenovectors not encoding a transgene (E) (empty adenovirus). Three weeks after the prime immunization animals were boost-immunized with MVA expressing the same antigens as during prime immunization (MVA.hPSMA.hSTEAP-1 at a dose of 1×10⁷ IU/mouse), while another group of mice was not boosted. Control animals were immunized at week 3 with MVA.hPSMA.hSTEAP-1 (at a dose of 1×10⁷ IU/mouse). Immune responses were measured at week 4 post prime immunization. Cells were stimulated overnight with peptide pools spanning a 15mer overlapping peptides spanning the hPSMA or hSTEAP-1 wild type antigen. The antigen specific immune responses were determined by measuring the relative number of IFNγ-secreting cells. The data showed that immunization of mice with either Ad26.hPSMA and Ad26.hSTEAP1 alone or Ad26.hPSMA, Ad26.hSTEAP1 and MVA.hPSMA.hSTEAP1 resulted in cellular immune responses against both proteins. In contrast, the induced immune response after a prime immunization with MVA.hPSMA.hSTEAP-1 was at the same level as that seen with the negative vaccine control Ad26.E. The overall response was highest in animals boost-immunized with MVA.hPSMA.hSTEAP1. FIG. 15 shows the study design of mice utilizing prime-boost vaccination of mice with Ad26.hPSMA, Ad26.hSTEAP1 and MVA.hPSMA.hSTEAP1. FIG. 16 shows the log of the number of IFNγ spot forming units (SFU) per 10⁶ splenocytes from splenocytes isolated from mice immunized with MVA.hPSMA.hSTEAP1 as a prime (Group1; Gr1), Ad26.hPSMA, Ad26.hSTEAP1 as a prime (Group 3, Gr3), Ad26.hPSMA, Ad26.hSTEAP1 as a prime and MVA.hPSMA.hSTEAP1 as a boost (Group 4, Gr4) or with an empty Ad26 vector (Ad26.Empty, Group 10, Gr10) and stimulated overnight with PSMA peptide pool. Group 5 prime-boost regimen significantly potentiated immune responses as measured by increased IFNγ production. FIG. 17 shows the log of the number of IFNγ spot forming units (SFU) per 10⁶ splenocytes from splenocytes isolated from mice immunized with MVA.hPSMA.hSTEAP1 as a prime (Group1; Gr1), Ad26.hPSMA, Ad26.hSTEAP1 as a prime (Group 3, Gr3), Ad26.hPSMA, Ad26.hSTEAP1 as a prime and MVA.hPSMA.hSTEAP1 as a boost (Group 4, Gr4) or with an empty Ad26 vector (Ad26.Empty, Group 10, Gr10) and stimulated overnight with STEAP1 peptide pool.

### Example 8. Prime-Boost Regiments of Ad26.hPSMA, Ad26.hSTEAP1 and MVA.hPSMA.hSTEAP1 in Combination with Anti-CTLA4 Antibodies Enhance Immune Responses in Non-Human Primates

The ability of prime-boost regimens optionally in combination with checkpoint inhibitors to potentiate in magnitude and duration of T cell responses vs. prime only was assessed in non-human primate models.

### Materials and Methods

Animals were prime immunized at week 0 with Ad26.hPSMA and Ad26.hSTEAP-1 using 5×10¹⁰ vp per adenovector. Vaccines were administered via intramuscular injection, into the quadriceps muscle (into one leg), alone or in combination with 1) 10 mg/kg Ipi IV, or 2) 3 mg/kg Ipi SC. Four weeks and eight weeks later animals were boost immunized intramuscularly, into the quadriceps muscle with MVA.hPSMA.hASTEAP-1, using 1×10⁸ TCID50/animals (into one leg) alone or in combination with 1) 10 mg/kg Ipi IV, or 2) 3 mg/kg Ipi SC. The group size of the study was six, eight or nine animals.

Induction of total (hPSMA+hSTEAP1) specific T-cell responses per 10⁶ PBMCs was measured over time. The total response was calculated per animal as follows: (PSMA response minus medium response) plus (STEAP1 response minus medium response). Values below 100 SFU/10⁶ cells were adjusted to 100 SFU/10⁶ cells. An ANOVA Tobit model with adjustment for potentially censored values was applied on log₁₀-transformed total SFU responses with group as explanatory factor. Statistical analysis was done per time point over the total response comparing Group 1 versus Group 2 (primary analysis, significance is shown by *, corresponding to p<0.005) or comparing Group 1 versus Group 3 or Group 4 (secondary analysis, significance is shown by # for Group 1 versus Group 3, corresponding to p=0.032) at the indicated time points.

### Results

Cynomolgus macaques were immunized IM with a combination of Ad26.hPSMA and Ad26.hSTEAP1 at a dose of 5×10¹⁰ VP of each vector alone or in combination with ipilimumab (abbreviated as Ipi in the Figures)(10 mg/kg intravenously [IV] or 3 mg/kg SC). Four weeks later, animals received a boost immunization with MVA.hPSMA.hSTEAP1 (10⁸ IU), alone or in combination with ipilimumab at 10 mg/kg IV or 3 mg/kg SC. At Week 8 animals received a second boost with the same material that was given at Week 4. Control animals were primed with Ad26.hPSMA and Ad26.hSTEAP1 but did not receive any boost immunizations or ipilimumab. The induction of immune responses to hPSMA or hSTEAP1 was evaluated in peripheral blood mononuclear cells (PBMCs; blood) at various time points during the study by IFN-γ Elispot.

Ad26.hPSMA and Ad26.hSTEAP1 prime immunization induced cellular immune responses at week 2 and week 4. Intravenously injection of Ipilimumab (group3) resulted in a 1.7-2.4-fold increase in the magnitude of total response compared to the response induced by Ad26.hPSMA and Ad26.hSTEAP1 only (group 1 and group 2), though it did not reach statistical significance. Minor effect was seen with subcutaneous injected ipilimumab.

A boost with MVA.hPSMA.hSTEAP1 at week 4 and at week 8 enhanced the total cellular immune response compared to that induced by a single prime immunization with Ad26, 2-6-3.9-fold (Tobit LRT, Bonferroni, week 6: p=0.005; week 8: p=0.004; week 10: p<0.001).

In animals that did not receive the ipilimumab A (group 1) a contraction of the immune response was seen at week 8 compared to week 6. In contrast, intravenous injection of Ipilimumab (group2) resulted in maintaining the magnitude of the response seen at week 6, which increased further after a 2^{nd} boost at week 8.

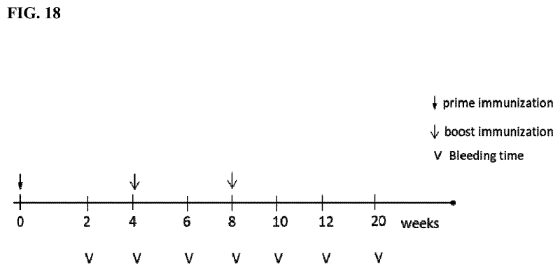

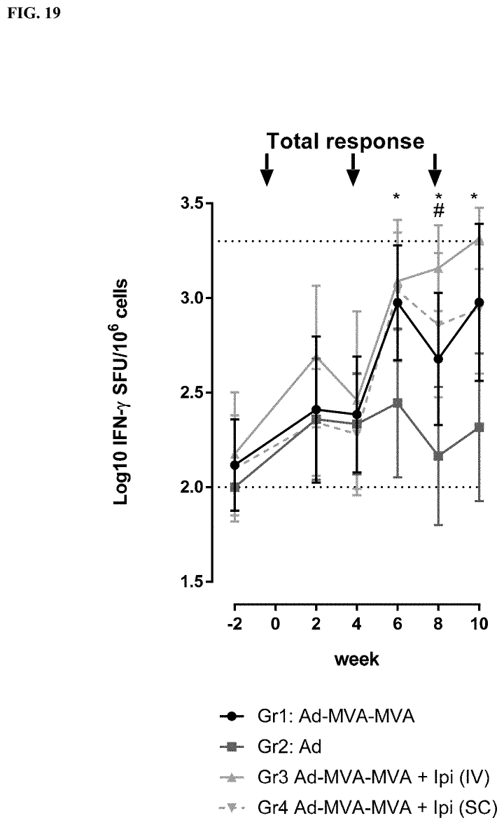

FIG. 18 shows the non-human primate prime-boost study dosing. FIG. 19 shows log of the number of IFNγ spot forming units (SFU) per 10⁶ splenocytes from splenocytes isolated over time as indicated in the figure from Cynomolgous macaques primed with Ad26.hPSMA and Ad26.hSTEAP1 and boosted at 4 weeks and at 8 weeks with MVA.hPSMA.hSTEAP1 (Group 1, Gr1), primed with Ad26.hPSMA and Ad26.hSTEAP1 without receiving boost (Group 2, Gr2), primed with Ad26.hPSMA and Ad26.hSTEAP1 and boosted at 4 weeks and at 8 weeks with MVA.hPSMA.hSTEAP1 and administered ipilimumab IV at both 4 weeks and 8 weeks (Group 3, Gr3), and primed with Ad26.hPSMA and Ad26.hSTEAP1 and boosted at 4 weeks and at 8 weeks with MVA.hPSMA.hSTEAP1 and administered ipilimumab SC at both 4 weeks and 8 weeks (Group 4, Gr4) stimulated overnight with hPSMA and hSTEAP1 peptide pools. The lower dotted line corresponds to the cut-off value of 100 SFU/10⁶ cells, whereas the upper dotted line corresponds to the upper limit of quantification (ULoQ). The error bars indicate standard deviation. The arrows refer to the time of immunization. An ANOVA Tobit model with adjustment for potentially censored values was applied on log₁₀-transformed total SFU responses with group as explanatory factor. Statistical analysis was done per time point over the total response comparing Group 1 versus Group 2 (primary analysis, significance is shown by *, corresponding to p<0.005) or comparing Group 1 versus Group 3 or Group 4 (secondary analysis, significance is shown by # for Group 1 versus Group 3, corresponding to p=0.032) at the indicated time points.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### EMBODIMENTS

The following list of embodiments is intended to complement, rather than displace or supersede, the previous descriptions.

Embodiment 1. A vaccine combination, comprising:

a) a first polynucleotide encoding PSMA;

b) a second polynucleotide encoding STEAP1; and

c) a third polynucleotide encoding PSMA and STEAP1.

Embodiment 2. The vaccine combination of embodiment 1, wherein a recombinant adenovirus (rAd), a Great Ape adenovirus 20 (GAd20), a modified vaccinia Ankara (rMVA), or a self-replicating RNA comprise the first, second, or third polynucleotides.
Embodiment 3. The vaccine combination of embodiment 1 or 2, wherein the rAd is a recombinant adenovirus serotype 26 (rAd26).
Embodiment 4. The vaccine combination of any one of the previous embodiments, wherein:
a) a rAd26 comprises the first polynucleotide;
b) a rAd26 comprises the second polynucleotide; and
c) a rMVA comprises the third polynucleotide.
   Embodiment 5. The vaccine combination of any one of the previous embodiments, wherein the first polynucleotide and the second polynucleotide further comprise an operator-containing promoter operably linked to the polynucleotide.
   Embodiment 6. The vaccine combination of embodiment 5, wherein the operator-containing promoter comprises a CMV promoter and a tetracyclin operon operator (TetO).
   Embodiment 7. The vaccine combination of embodiment 5 or 6, wherein the operator-containing promoter comprises the polynucleotide of SEQ ID NO: 20.
   Embodiment 8. The vaccine combination of any one of the previous embodiments, wherein the first polynucleotide and the second polynucleotide further comprise a SV40 polyadenylation signal (SV40 pA).
   Embodiment 9. The vaccine combination of embodiment 8, wherein the SV40 pA comprises the polynucleotide of SEQ ID NO: 21.
   Embodiment 10. The vaccine combination of any one of the previous embodiments, wherein:
a) the polynucleotide encoding PSMA encodes the polypeptide of SEQ ID NO: 15; and/or
b) the polynucleotide encoding PSMA comprises the polynucleotide of SEQ ID NO: 14.
   Embodiment 11. The vaccine combination of any one of the previous embodiments, wherein the first polynucleotide comprises:
a) the polynucleotide encoding the polypeptide of SEQ ID NO: 15; and/or
b) the polynucleotide of SEQ ID NO: 16.
   Embodiment 12. The vaccine combination of any one of the previous embodiments, wherein:
a) the polynucleotide encoding STEAP1 encodes a polypeptide of SEQ ID NO: 18; and/or
b) the polynucleotide encoding STEAP1 comprises the polynucleotide of SEQ ID NO: 17.
   Embodiment 13. The vaccine combination of any one of the previous embodiments, wherein the second polynucleotide comprises:
a) the polynucleotide encoding the polypeptide of SEQ ID NO: 18, and/or
b) the polynucleotide of SEQ ID NO: 19.
   Embodiment 14. The vaccine combination of any one of the previous embodiments, wherein the first polynucleotide and the second polynucleotide are inserted into rAd26 E1 deletion site.
   Embodiment 15. The vaccine combination of any one of the previous embodiments, wherein the third polynucleotide further comprises a poxvirus promoter operably linked to the polynucleotide.
   Embodiment 16. The vaccine combination of embodiment 15, wherein the poxvirus promoter comprises a vaccinia virus promoter p7.5 of SEQ ID NO: 1.
   Embodiment 17. The vaccine combination of any one of the previous embodiments, wherein the third polynucleotide further comprises a polynucleotide encoding a first T cell enhancer (TCE) and a polynucleotide encoding a second TCE.
   Embodiment 18. The vaccine combination of embodiment 17, wherein the polynucleotide encoding the first TCE encodes the polypeptide of SEQ ID NO: 13 and the polynucleotide encoding the second TCE encodes the polypeptide of SEQ ID NO: 7.
   Embodiment 19. The vaccine combination of embodiment 18, wherein the polynucleotide encoding the first TCE comprises the polynucleotide of SEQ ID NO: 2 and/or the polynucleotide encoding the second TCE comprises the polynucleotide of SEQ ID NO: 5.
   Embodiment 20. The vaccine combination of any one of the previous embodiments, wherein the third polynucleotide further comprises a polynucleotide encoding a 2A self-cleaving peptide.
   Embodiment 21. The vaccine combination of embodiment 20, wherein the polynucleotide encoding the 2A self-cleaving peptide encodes the polypeptide of SEQ ID NO: 9.
   Embodiment 22. The vaccine combination of embodiment 21, wherein the polynucleotide encoding the 2A self-cleaving peptide comprises the polynucleotide of SEQ ID NO: 4.
   Embodiment 23. The vaccine combination of any one of the previous embodiments, wherein in the third polynucleotide:
a) the polynucleotide encoding PSMA encodes the polypeptide of SEQ ID NO: 8;
b) the polynucleotide encoding PSMA comprises the polynucleotide of SEQ ID NO: 3;
c) the polynucleotide encoding STEAP1 encodes the polypeptide of SEQ ID NO: 10; and/or
d) the polynucleotide encoding STEAP1 comprises the polynucleotide of SEQ ID NO: 6.
   Embodiment 24. The vaccine combination any one of the previous embodiments, wherein in the third polynucleotide:
a) the polynucleotide encoding PSMA is located 5′ to the polynucleotide encoding STEAP1;
b) a poxvirus promoter is located 5′ to the polynucleotide encoding PSMA;
c) a polynucleotide encoding a first TCE is located 5′ to the polynucleotide encoding PSMA;
d) a polynucleotide encoding a second TCE is located 3′ to the polynucleotide encoding PSMA; and/or
e) a polynucleotide encoding a 2A self-cleaving peptide is located 3′ to the polynucleotide encoding PSMA and 5′ to the polynucleotide encoding the second TCE.
   Embodiment 25. The vaccine combination of any one of the previous embodiments, wherein the third polynucleotide comprises:
a) the polynucleotide encoding the polypeptide of SEQ ID NO: 12; and/or
b) the polynucleotide of SEQ ID NO: 11.
   Embodiment 26. The vaccine combination of any one of the previous embodiments, wherein the rMVA is derived from MVA-476 MG/14/78, MVA-572, MVA-574 or MVA-575 or MVA-BN.
   Embodiment 27. The vaccine combination of any one of the previous embodiments, wherein the third polynucleotide is inserted into a rMVA deletion site III.
   Embodiment 28. A recombinant adenovirus comprising a polynucleotide encoding PSMA.
   Embodiment 29. The recombinant adenovirus of embodiment 28, wherein the polynucleotide further comprises an operator-containing promoter operably linked to the polynucleotide encoding PSMA.
   Embodiment 30. The recombinant adenovirus of embodiment 29, wherein the operator-containing promoter comprises a CMV promoter and a tetracyclin operon operator (TetO).
   Embodiment 31. The recombinant adenovirus of embodiment 30, wherein the operator-containing promoter comprises the polynucleotide of SEQ ID NO: 20.
   Embodiment 32. The recombinant adenovirus of any one of embodiments 28-31, wherein the polynucleotide further comprises a SV40 pA signal.
   Embodiment 33. The recombinant adenovirus of embodiment 32, wherein the SV40 pA comprises the polynucleotide of SEQ ID NO: 21.
   Embodiment 34. The recombinant adenovirus of any one of embodiments 28-33, wherein:
a) the polynucleotide encoding PSMA encodes the polypeptide of SEQ ID NO: 15; and/or
b) the polynucleotide encoding PSMA comprises the polynucleotide of SEQ ID NO: 14.
   Embodiment 35. The recombinant adenovirus of any one of embodiments 28-34, wherein:
a) the polynucleotide encodes the polypeptide of SEQ ID NO: 15; and/or
b) the polynucleotide comprises the sequence of SEQ ID NO: 16.
   Embodiment 36. The recombinant adenovirus of any one of embodiments 28-35, wherein the recombinant adenovirus is derived from a human adenovirus serotype 26 (Ad26).
   Embodiment 37. The recombinant adenovirus of any one of embodiments 28-36, wherein the polynucleotide is inserted into an E1 deletion site or into an E3 deletion site.
   Embodiment 38. A polynucleotide comprising the sequence of SEQ ID NO: 16.
   Embodiment 39. A vector comprising the polynucleotide of embodiment 38.
   Embodiment 40. A cell comprising the vector of embodiment 39.
   Embodiment 41. A cell comprising the recombinant adenovirus of any one of embodiments 28-37.
   Embodiment 42. A recombinant adenovirus (rAd) comprising a polynucleotide encoding STEAP1.
   Embodiment 43. The recombinant adenovirus of embodiment 42, wherein the polynucleotide further comprises an operator-containing promoter operably linked to the polynucleotide.
   Embodiment 44. The recombinant adenovirus of embodiment 43, wherein the operator-containing promoter comprises a CMV promoter and a tetracyclin operon operator (TetO).
   Embodiment 45. The recombinant adenovirus of embodiment 44, wherein the operator-containing promoter comprises the polynucleotide of SEQ ID NO: 20.
   Embodiment 46. The recombinant adenovirus of any one of embodiments 42-45, wherein the polynucleotide further comprises a SV40 pA signal.
   Embodiment 47. The recombinant adenovirus of embodiment 46, wherein the SV40 pA comprises the polynucleotide of SEQ ID NO: 21.
   Embodiment 48. The recombinant adenovirus of any one of embodiments 42-47, wherein:
a) the polynucleotide encoding STEAP1 encodes the polypeptide of SEQ ID NO: 18; and/or
b) the polynucleotide encoding STEAP1 comprises the polynucleotide of SEQ ID NO: 17.
   Embodiment 49. The recombinant adenovirus of any one of embodiments 42-48, wherein:
a) the polynucleotide encodes the polypeptide of SEQ ID NO: 18; and/or
b) the polynucleotide comprises the sequence of SEQ ID NO: 19.
   Embodiment 50. The recombinant adenovirus of any one of embodiments 42-49, wherein the recombinant adenovirus is derived from a human adenovirus serotype 26 (Ad26).
   Embodiment 51. The recombinant adenovirus of any one of embodiments 42-50, wherein the polynucleotide is inserted into an E1 deletion site or into an E3 deletion site.
   Embodiment 52. A polynucleotide comprising the sequence of SEQ ID NO: 19.
   Embodiment 53. A vector comprising the polynucleotide of embodiment 52.
   Embodiment 54. A cell comprising the vector of embodiment 53.
   Embodiment 55. A cell comprising the recombinant adenovirus of any one of embodiments 42-51.
   Embodiment 56. A recombinant modified vaccinia Ankara (rMVA) virus comprising a polynucleotide encoding PSMA and STEAP1.
   Embodiment 57. The recombinant modified vaccinia Ankara virus of embodiment 56, wherein the polynucleotide further comprises a poxvirus promoter operably linked to the polynucleotide.
   Embodiment 58. The recombinant modified vaccinia Ankara virus of embodiment 57, wherein the poxvirus promoter is a vaccinia virus promoter p7.5 comprising the polynucleotide of SEQ ID NO: 1.
   Embodiment 59. The recombinant modified vaccinia Ankara virus of any one of embodiments 56-58, wherein the polynucleotide further comprises a polynucleotide encoding a first T cell enhancer (TCE) and a polynucleotide encoding a second TCE.
   Embodiment 60. The recombinant modified vaccinia Ankara virus of embodiment 59, wherein the polynucleotide encoding the first TCE encodes the polypeptide of SEQ ID NO: 13 and the polynucleotide encoding the second TCE encodes the polypeptide of SEQ ID NO: 7.
   Embodiment 61. The recombinant modified vaccinia Ankara virus of embodiment 60, wherein the polynucleotide encoding the first TCE comprises the polynucleotide of SEQ ID NO: 2 and/or the polynucleotide encoding the second TCE comprises the polynucleotide of SEQ ID NO: 5.
   Embodiment 62. The recombinant modified vaccinia Ankara virus of any one of embodiments 56-61, wherein the polynucleotide further comprises a polynucleotide encoding a 2A self-cleaving peptide.
   Embodiment 63. The recombinant modified vaccinia Ankara virus of embodiment 62, wherein the polynucleotide encoding the 2A self-cleaving peptide encodes the polypeptide of SEQ ID NO: 9.
   Embodiment 64. The recombinant modified vaccinia Ankara virus of embodiment 63, wherein the polynucleotide encoding the 2A self-cleaving peptide comprises the polynucleotide of SEQ ID NO: 4.
   Embodiment 65. The recombinant modified vaccinia Ankara virus of any one of embodiments 56-64, wherein:
a) the polynucleotide encoding PSMA encodes the polypeptide of SEQ ID NO: 8;
b) the polynucleotide encoding PSMA comprises the polynucleotide of SEQ ID NO: 3;
c) the polynucleotide encoding STEAP1 encodes the polypeptide of SEQ ID NO: 10; and/or
d) the polynucleotide encoding STEAP1 comprises the polynucleotide of SEQ ID NO: 6.
   Embodiment 66. The recombinant modified vaccinia Ankara virus of any one of embodiments 56-65, wherein:
a) the polynucleotide encoding PSMA is located 5′ to the polynucleotide encoding STEAP1;
b) a poxvirus is located 5′ to the polynucleotide encoding PSMA;
c) a polynucleotide encoding a first TCE is located 5′ to the polynucleotide encoding PSMA;
d) a polynucleotide encoding a second TCE is located 3′ to the polynucleotide encoding PSMA; and/or
e) a polynucleotide encoding a 2A self-cleaving peptide is located 3′ to the polynucleotide encoding PSMA and 5′ to the polynucleotide encoding the second TCE.
   Embodiment 67. The recombinant modified vaccinia Ankara virus of any one of any one of embodiments 56-66, wherein:
a) the polynucleotide encodes the polypeptide of SEQ ID NO: 12; and/or
b) the polynucleotide comprises the sequence of SEQ ID NO: 11.
   Embodiment 68. The recombinant modified vaccinia Ankara virus of any one of embodiments 56-67, wherein the recombinant modified vaccinia Ankara is derived from MVA-476 MG/14/78, MVA-572, MVA-574 or MVA-575 or MVA-BN.
   Embodiment 69. The recombinant modified vaccinia Ankara virus of any one of embodiments 56-68, wherein the polynucleotide is inserted into a deletion site III.
   Embodiment 70. A polynucleotide comprising the sequence of SEQ ID NO: 12.
   Embodiment 71. A polynucleotide comprising the sequence of SEQ ID NO: 11.
   Embodiment 72. A vector comprising the polynucleotide of embodiment 70 or 71.
   Embodiment 73. A cell comprising the vector of embodiment 72.
   Embodiment 74. A cell comprising the recombinant MVA of any one of embodiments 56-69.
   Embodiment 75. A method of enhancing an immune response against a prostate cancer in a subject afflicted with the prostate cancer, comprising administering to the subject the vaccine combination of any one of embodiments 1-27.
   Embodiment 76. A method of enhancing an immune response against a prostate cancer in a subject in a subject in need thereof, comprising administering to the subject
a) an immunologically effective amount of a first recombinant adenovirus serotype 26 (Ad26) virus comprising a first polynucleotide encoding PSMA for priming the immune response;
b) an immunologically effective amount of a second recombinant Ad26 virus comprising a second polynucleotide encoding STEAP1 for priming the immune response; and
c) an immunologically effective amount of a recombinant modified vaccinia Ankara (MVA) virus comprising a third polynucleotide encoding PSMA and STEAP1 for boosting the immune response.
   Embodiment 77. A method of treating a subject afflicted with a prostate cancer, comprising administering to the subject:
a) an immunologically effective amount of a first recombinant adenovirus serotype 26 (Ad26) virus comprising a first polynucleotide encoding PSMA for priming the immune response;
b) an immunologically effective amount of a second recombinant Ad26 virus comprising a second polynucleotide encoding STEAP1 for priming the immune response; and
c) an immunologically effective amount of a recombinant modified vaccinia Ankara (MVA) virus comprising a third polynucleotide encoding PSMA and STEAP1 for boosting the immune response.
   Embodiment 78. The method of any one of embodiments 75-77, wherein the first recombinant Ad26, the second recombinant Ad26 and the recombinant MVA are formulated in a pharmaceutical composition.
   Embodiment 79. The method of any one of embodiments 75-78, wherein the immune response is a CD8+ T cell response or a CD4+ T cell response.
   Embodiment 80. The method of any one of embodiments 75-79, wherein the first recombinant Ad26 comprises Ad26.PSMA, the second recombinant Ad26 comprises Ad26.STEAP1, and the recombinant MVA comprises MVA.PSMA.STEAP1.
   Embodiment 81. The method of any one of embodiments 75-80, further comprising administering one or more additional cancer therapeutics.
   Embodiment 82. The method of embodiment 81, wherein the one or more additional cancer therapeutics is a surgery, a chemotherapy, an androgen deprivation therapy, radiation therapy, targeted therapy or a checkpoint inhibitor, or any combination thereof
   Embodiment 83. The method of embodiment 82, wherein the checkpoint inhibitor is an inhibitor of CTLA-4, an inhibitor of PD-1, or an inhibitor of PD-L1.
   Embodiment 84. A pharmaceutical composition comprising the rAd, the rMVA, the vaccine combination, the polynucleotide, the polypeptide, the vector, or the cell of any one of embodiments 1-74.

What is claimed:

1. A vaccine combination, comprising:
a) a first polynucleotide encoding PSMA;
b) a second polynucleotide encoding STEAP1; and
c) a third polynucleotide encoding PSMA and STEAP1.

2. The vaccine combination of claim 1, wherein a recombinant adenovirus (rAd), Great Ape adenovirus 20 (GAd20), a modified vaccinia Ankara (rMVA), or a self-replicating RNA comprise the first, second, or third polynucleotides.

3. The vaccine combination of claim 2, wherein the rAd is a recombinant adenovirus serotype 26 (rAd26).

4. The vaccine combination of claim 1, wherein:
a) a rAd26 comprises the first polynucleotide;
b) a rAd26 comprises the second polynucleotide; and
c) a rMVA comprises the third polynucleotide.

5. The vaccine combination of claim 1, wherein:
a) the polynucleotide encoding PSMA encodes the polypeptide of SEQ ID NO: 15; and/or
b) the polynucleotide encoding PSMA comprises the polynucleotide of SEQ ID NO: 14.

6. The vaccine combination of claim 1, wherein the first polynucleotide comprises:
a) the polynucleotide encoding the polypeptide of SEQ ID NO: 15; and/or
b) the polynucleotide of SEQ ID NO: 16.

7. The vaccine combination of claim 1, wherein:
a) the polynucleotide encoding STEAP1 encodes a polypeptide of SEQ ID NO: 18; and/or
b) the polynucleotide encoding STEAP1 comprises the polynucleotide of SEQ ID NO: 17.

8. The vaccine combination of claim 1, wherein the second polynucleotide comprises:
a) the polynucleotide encoding the polypeptide of SEQ ID NO: 18, and/or
b) the polynucleotide of SEQ ID NO: 19.

9. The vaccine combination of claim 1, wherein in the third polynucleotide:
a) the polynucleotide encoding PSMA encodes the polypeptide of SEQ ID NO: 8;
b) the polynucleotide encoding PSMA comprises the polynucleotide of SEQ ID NO: 3;
c) the polynucleotide encoding STEAP1 encodes the polypeptide of SEQ ID NO: 10; and/or
d) the polynucleotide encoding STEAP1 comprises the polynucleotide of SEQ ID NO: 6.

10. The vaccine combination of claim 1, wherein in the third polynucleotide:
a) the polynucleotide encoding PSMA is located 5′ to the polynucleotide encoding STEAP1;
b) a poxvirus promoter is located 5′ to the polynucleotide encoding PSMA;
c) a polynucleotide encoding a first TCE is located 5′ to the polynucleotide encoding PSMA;
d) a polynucleotide encoding a second TCE is located 3′ to the polynucleotide encoding PSMA; and/or
e) a polynucleotide encoding a 2A self-cleaving peptide is located 3′ to the polynucleotide encoding PSMA and 5′ to the polynucleotide encoding the second TCE.

11. The vaccine combination of claim 1, wherein the third polynucleotide comprises:
a) the polynucleotide encoding the polypeptide of SEQ ID NO: 12; and/or
b) the polynucleotide of SEQ ID NO: 11.

12. The vaccine combination of claim 2, wherein the rMVA is derived from MVA-476 MG/14/78, MVA-572, MVA-574 or MVA-575 or MVA-BN.

13. A polynucleotide encoding PSMA, wherein:
a) the polynucleotide encodes the polypeptide of SEQ ID NO: 15; and/or
b) the polynucleotide comprises the polynucleotide of SEQ ID NO: 14.

14. The polynucleotide of claim 13, wherein:
a) the polynucleotide encodes the polypeptide of SEQ ID NO: 15; and/or
b) the polynucleotide comprises the sequence of SEQ ID NO: 16.

15. A vector comprising the polynucleotide of claim 13.

16. The vector of claim 15, wherein the vector comprises a recombinant adenovirus (rAd), Great Ape adenovirus 20 (GAd20), a modified vaccinia Ankara (rMVA), or a self-replicating RNA.

17. The vector of claim 16, wherein the vector comprises a recombinant adenovirus derived from a human adenovirus serotype 26 (Ad26).

18. A cell comprising the vector of claim 16.

19. A polynucleotide encoding STEAP1, wherein:
a) the polynucleotide encodes the polypeptide of SEQ ID NO: 18; and/or
b) the polynucleotide comprises the polynucleotide of SEQ ID NO: 17.

20. The polynucleotide of claim 19, wherein:
a) the polynucleotide encodes the polypeptide of SEQ ID NO: 18; and/or
b) the polynucleotide comprises the sequence of SEQ ID NO: 19.

21. A vector comprising the polynucleotide of claim 19.

22. The vector of claim 21, wherein the vector comprises a recombinant adenovirus (rAd), Great Ape adenovirus 20 (GAd20), a modified vaccinia Ankara (rMVA), or a self-replicating RNA.

23. The vector of claim 22, wherein the vector comprises a recombinant adenovirus derived from a human adenovirus serotype 26 (Ad26).

24. A cell comprising the vector of claim 22.

25. A polynucleotide encoding PSMA and STEAP1, wherein:
a) the portion of the polynucleotide encoding PSMA encodes the polypeptide of SEQ ID NO: 8;
b) the portion of the polynucleotide encoding PSMA comprises the polynucleotide of SEQ ID NO: 3;
c) the portion of the polynucleotide encoding STEAP1 encodes the polypeptide of SEQ ID NO: 10; and/or
d) the portion of the polynucleotide encoding STEAP1 comprises the polynucleotide of SEQ ID NO: 6.

26. The polynucleotide of claim 25, wherein:
a) the portion of the polynucleotide encoding PSMA is located 5′ to the portion of the polynucleotide encoding STEAP1;
b) a poxvirus promoter is located 5′ to the portion of the polynucleotide encoding PSMA;
c) a polynucleotide encoding a first TCE is located 5′ to the portion of the polynucleotide encoding PSMA;
d) a polynucleotide encoding a second TCE is located 3′ to the portion of the polynucleotide encoding PSMA; and/or
e) a polynucleotide encoding a 2A self-cleaving peptide is located 3′ to the portion of the polynucleotide encoding PSMA and 5′ to the polynucleotide encoding the second TCE.

27. The polynucleotide of claim 25, wherein:
a) the polynucleotide encodes the polypeptide of SEQ ID NO: 12; and/or
b) the polynucleotide comprises the sequence of SEQ ID NO: 11.

28. A vector comprising the polynucleotide of claim 25.

29. The vector of claim 28, wherein the vector comprises a recombinant adenovirus (rAd), Great Ape adenovirus 20 (GAd20), a modified vaccinia Ankara (rMVA), or a self-replicating RNA.

30. The vector of claim 29, wherein the vector comprises a recombinant modified vaccinia Ankara (rMVA).

31. A cell comprising the vector of claim 29.

32. A method of enhancing an immune response against a prostate cancer in a subject afflicted with the prostate cancer, comprising administering to the subject the vaccine combination of claim 1.

33. A method of enhancing an immune response against a prostate cancer in a subject in need thereof, comprising administering to the subject
a) an immunologically effective amount of a first recombinant adenovirus serotype 26 (Ad26) virus comprising a first polynucleotide encoding PSMA for priming the immune response;
b) an immunologically effective amount of a second recombinant Ad26 virus comprising a second polynucleotide encoding STEAP1 for priming the immune response; and
c) an immunologically effective amount of a recombinant modified vaccinia Ankara (MVA) virus comprising a third polynucleotide encoding PSMA and STEAP1 for boosting the immune response.

34. A method of treating a subject afflicted with a prostate cancer, comprising administering to the subject:
a) an immunologically effective amount of a first recombinant adenovirus serotype 26 (Ad26) virus comprising a first polynucleotide encoding PSMA for priming the immune response;
b) an immunologically effective amount of a second recombinant Ad26 virus comprising a second polynucleotide encoding STEAP1 for priming the immune response; and
c) an immunologically effective amount of a recombinant modified vaccinia Ankara (MVA) virus comprising a third polynucleotide encoding PSMA and STEAP1 for boosting the immune response.

35. The method of claim 32, further comprising administering one or more additional cancer therapeutics.

36. The method of claim 35, wherein the one or more additional cancer therapeutics is a surgery, a chemotherapy, an androgen deprivation therapy, radiation therapy, targeted therapy or a checkpoint inhibitor, or any combination thereof.

37. The method of claim 36, wherein the checkpoint inhibitor is an inhibitor of CTLA-4, an inhibitor of PD-1, or an inhibitor of PD-L1.

38. A pharmaceutical composition comprising the vaccine combination of claim 1.


SEQUENCE LISTING (34 sequences)

SEQ ID NO: 1 - DNA, 140 bp, Vaccinia virus
gatcactaattccaaacccacccgctttttatagtaagtttttcacccataaataataaatacaataattaatttctcgtaaaagtagaaaatatattctaatttattgcacggtaaggaagtagaatcataaagaacag

SEQ ID NO: 2 - DNA, 84 bp, Artificial Sequence
  Description of Artificial Sequence Synthetic oligonucleotide
atgggccagaaggaacagattcatacgcttcagaaaaattctgaacgaatgtcaaagcaattgacacgaagttctcaggcagta

SEQ ID NO: 3 - DNA, 2247 bp, Homo sapiens
tggaatctccttcacgaaaccgactcggctgtggctaccgcacgcagacctaggtggctgtgtgctggagctctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcactcttctctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggccattctataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcc

SEQ ID NO: 4 - DNA, 72 bp, Artificial Sequence
  Description of Artificial Sequence Synthetic oligonucleotide
gcgccagtaaagcagacattaaactttgatttgctgaaacttgcaggtgatgtagagtcaaatccaggtcca

SEQ ID NO: 5 - DNA, 81 bp, Artificial Sequence
  Description of Artificial Sequence Synthetic oligonucleotide
ggacagaaagagcaaatccacacactgcagaagaacagcgagaggatgagcaaacagcttaccaggtcatcccaagctgtt

SEQ ID NO: 6 - DNA, 1014 bp, Homo sapiens
gaaagcagaaaagacatcacaaaccaagaagaactttggaaaatgaagcctaggagaaatttagaagaagacgattatttgcataaggacacgggagagaccagcatgctaaaaagacctgtgcttttgcatttgcaccaaacagcccatgctgatgaatttgactgcccttcagaacttcagcacacacaggaactctttccacagtggcacttgccaattaaaatagctgctattatagcatctctgacttttctttacactcttctgagggaagtaattcaccctttagcaacttcccatcagcaatacttctataagattccaatcctggtcatcaacaaagtcttgccaatggtttccatcactctcttggcattggtttacctgccaggtgtgatagcagcaattgtccaacttcataatggaaccaagtataagaagtttccacattggttggataagtggatgttaacaagaaagcagtttgggcttctcagtttcttctttgctgtactgcatgcaatttatagtctgtcttacccaatgaggcgatcctacagatacaagttgctaaactgggcatatcaacaggtccaacaaaataaagaagatgcctggattgagcatgatgtttggagaatggagatttatgtgtctctgggaattgtgggattggcaatactggctctgttggctgtgacatctattccatctgtgagtgactctttgacatggagagaatttcactatattcagagcaagctaggaattgtttcccttctactgggcacaatacacgcattgatttttgcctggaataagtggatagatataaaacaatttgtatggtatacacctccaacttttatgatagctgttttccttccaattgttgtcctgatatttaaaagcatactattcctgccatgcttgaggaagaagatactgaagattagacatggttgggaagacgtcaccaaaattaacaaaactgagatatgttcccagttg

SEQ ID NO: 7 - protein, 27 aa, Siniperca chuatsi
GQKEQIHTLQKNSERMSKQLTRSSQAV

SEQ ID NO: 8 - protein, 749 aa, Homo sapiens
WNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 9 - protein, 24 aa, Foot-and-mouth disease virus
APVKQTLNFDLLKLAGDVESNPGP

SEQ ID NO: 10 - protein, 338 aa, Homo sapiens
ESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 11 - DNA, 3505 bp, Artificial Sequence
  Description of Artificial Sequence Synthetic polynucleotide
atgggccagaaggaacagattcatacgcttcagaaaaattctgaacgaatgtcaaagcaattgacacgaagttctcaggcagtatggaatctccttcacgaaaccgactcggctgtggctaccgcacgcagacctaggtggctgtgtgctggagctctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcactcttctctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggccattctataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagccgcgccagtaaagcagacattaaactttgatttgctgaaacttgcaggtgatgtagagtcaaatccaggtccaggacagaaagagcaaatccacacactgcagaagaacagcgagaggatgagcaaacagcttaccaggtcatcccaagctgttgaaagcagaaaagacatcacaaaccaagaagaactttggaaaatgaagcctaggagaaatttagaagaagacgattatttgcataaggacacgggagagaccagcatgctaaaaagacctgtgcttttgcatttgcaccaaacagcccatgctgatgaatttgactgcccttcagaacttcagcacacacaggaactctttccacagtggcacttgccaattaaaatagctgctattatagcatctctgacttttctttacactcttctgagggaagtaattcaccctttagcaacttcccatcagcaatacttctataagattccaatcctggtcatcaacaaagtcttgccaatggtttccatcactctcttggcattggtttacctgccaggtgtgatagcagcaattgtccaacttcataatggaaccaagtataagaagtttccacattggttggataagtggatgttaacaagaaagcagtttgggcttctcagtttcttctttgctgtactgcatgcaatttatagtctgtcttacccaatgaggcgatcctacagatacaagttgctaaactgggcatatcaacaggtccaacaaaataaagaagatgcctggattgagcatgatgtttggagaatggagatttatgtgtctctgggaattgtgggattggcaatactggctctgttggctgtgacatctattccatctgtgagtgactctttgacatggagagaatttcactatattcagagcaagctaggaattgtttcccttctactgggcacaatacacgcattgatttttgcctggaataagtggatagatataaaacaatttgtatggtatacacctccaacttttatgatagctgttttccttccaattgttgtcctgatatttaaaagcatactattcctgccatgcttgaggaagaagatactgaagattagacatggttgggaagacgtcaccaaaattaacaaaactgagatatgttcccagttgtagtaaa

SEQ ID NO: 12 - protein, 1166 aa, Artificial Sequence
  Description of Artificial Sequence Synthetic polypeptide
MGQKEQIHTLQKNSERMSKQLTRSSQAVWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVAAPVKQTLNFDLLKLAGDVESNPGPGQKEQIHTLQKNSERMSKQLTRSSQAVESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 13 - protein, 28 aa, Siniperca chuatsi
MGQKEQIHTLQKNSERMSKQLTRSSQAV

SEQ ID NO: 14 - DNA, 2250 bp, Artificial Sequence
  Description of Artificial Sequence Synthetic polynucleotide
atgtggaacctgctgcacgagacagacagcgccgtggccacagcccggcggcccaggtggctgtgcgcaggcgccctggtgctggcaggaggcttctttctgctgggcttcctgtttggctggtttatcaagagcagcaacgaggccaccaatatcacacctaagcacaatatgaaggccttcctggacgagctgaaggccgagaatatcaagaagttcctgtacaactttacccagatcccacacctggccggcacagagcagaactttcagctggccaagcagatccagagccagtggaaggagttcggcctggactccgtggagctggcccactacgacgtgctgctgtcttatccaaataagacccaccccaactatatcagcatcatcaacgaggacggcaacgagattttcaacacatctctgtttgagccccctccacccggctacgagaacgtgagcgacatcgtgcctccattctctgcctttagcccacagggaatgcctgagggcgatctggtgtacgtgaattacgccaggaccgaggacttctttaagctggagcgcgatatgaagatcaactgtagcggcaagatcgtgatcgcccggtacggcaaggtgtttagaggcaataaggtgaagaacgcacagctggcaggagcaaagggcgtgatcctgtacagcgaccccgccgattatttcgcccctggcgtgaagtcctatccagacggctggaatctgccaggaggaggagtgcagaggggaaacatcctgaacctgaacggagcaggcgatcctctgaccccaggctaccccgccaacgagtacgcctataggaggggaatcgcagaggcagtgggcctgccttccatcccagtgcaccccatcggctactacgacgcccagaagctgctggagaagatgggaggctctgccccacctgattctagctggagaggcagcctgaaggtgccttacaacgtgggcccaggcttcaccggcaacttttccacacagaaggtgaagatgcacatccactctaccaacgaggtgacaaggatctataacgtgatcggcaccctgaggggagcagtggagcctgacagatacgtgatcctgggaggacacagggacagctgggtgtttggaggaatcgatccacagtccggagccgccgtggtgcacgagatcgtgcggtccttcggcaccctgaagaaggaggggtggcggccccggagaacaatcctgtttgcctcttgggacgccgaggagttcggcctgctgggctccacagagtgggcagaggagaacagccggctgctccaggagaggggagtggcctacatcaacgccgactcctctatcgagggcaactataccctgcgggtggattgcacacccctgatgtactccctggtgcacaacctgaccaaggagctgaagtctcctgacgagggcttcgagggcaagtctctgtacgagagctggacaaagaagtctccaagccccgagtttagcggcatgcctcggatctccaagctgggctctggcaacgatttcgaggtgttctttcagagactgggaatcgcatccggcagggcccgctacaccaagaattgggagacaaacaagttctctggctacccactgtatcacagcgtgtacgagacatacgagctggtggagaagttctacgaccccatgtttaagtatcacctgacagtggcacaggtgaggggaggaatggtgtttgagctggccaatagcatcgtgctgccattcgactgtcgggattacgccgtggtgctgagaaagtacgccgacaaaatctactccatctctatgaagcacccccaggagatgaagacctacagcgtgtccttcgattccctgttttctgccgtgaagaacttcacagagatcgccagcaagttttccgagcggctccaggacttcgataagtccaatcccatcgtgctgaggatgatgaacgaccagctgatgttcctggagcgcgcctttatcgaccctctgggcctgcctgatcggcccttctacagacacgtgatctacgcccctagctcccacaacaagtacgccggcgagtcttttccaggcatctacgacgccctgttcgatatcgagagcaaggtggacccctccaaggcctggggagaggtgaagagacaaatctacgtggcagccttcaccgtgcaggctgcagccgagacactgtccgaggtggcc

SEQ ID NO: 15 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 16 - DNA, 3292 bp, Artificial Sequence
  Description of Artificial Sequence Synthetic polynucleotide
tcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggatctagagccaccatgtggaacctgctgcacgagacagacagcgccgtggccacagcccggcggcccaggtggctgtgcgcaggcgccctggtgctggcaggaggcttctttctgctgggcttcctgtttggctggtttatcaagagcagcaacgaggccaccaatatcacacctaagcacaatatgaaggccttcctggacgagctgaaggccgagaatatcaagaagttcctgtacaactttacccagatcccacacctggccggcacagagcagaactttcagctggccaagcagatccagagccagtggaaggagttcggcctggactccgtggagctggcccactacgacgtgctgctgtcttatccaaataagacccaccccaactatatcagcatcatcaacgaggacggcaacgagattttcaacacatctctgtttgagccccctccacccggctacgagaacgtgagcgacatcgtgcctccattctctgcctttagcccacagggaatgcctgagggcgatctggtgtacgtgaattacgccaggaccgaggacttctttaagctggagcgcgatatgaagatcaactgtagcggcaagatcgtgatcgcccggtacggcaaggtgtttagaggcaataaggtgaagaacgcacagctggcaggagcaaagggcgtgatcctgtacagcgaccccgccgattatttcgcccctggcgtgaagtcctatccagacggctggaatctgccaggaggaggagtgcagaggggaaacatcctgaacctgaacggagcaggcgatcctctgaccccaggctaccccgccaacgagtacgcctataggaggggaatcgcagaggcagtgggcctgccttccatcccagtgcaccccatcggctactacgacgcccagaagctgctggagaagatgggaggctctgccccacctgattctagctggagaggcagcctgaaggtgccttacaacgtgggcccaggcttcaccggcaacttttccacacagaaggtgaagatgcacatccactctaccaacgaggtgacaaggatctataacgtgatcggcaccctgaggggagcagtggagcctgacagatacgtgatcctgggaggacacagggacagctgggtgtttggaggaatcgatccacagtccggagccgccgtggtgcacgagatcgtgcggtccttcggcaccctgaagaaggaggggtggcggccccggagaacaatcctgtttgcctcttgggacgccgaggagttcggcctgctgggctccacagagtgggcagaggagaacagccggctgctccaggagaggggagtggcctacatcaacgccgactcctctatcgagggcaactataccctgcgggtggattgcacacccctgatgtactccctggtgcacaacctgaccaaggagctgaagtctcctgacgagggcttcgagggcaagtctctgtacgagagctggacaaagaagtctccaagccccgagtttagcggcatgcctcggatctccaagctgggctctggcaacgatttcgaggtgttctttcagagactgggaatcgcatccggcagggcccgctacaccaagaattgggagacaaacaagttctctggctacccactgtatcacagcgtgtacgagacatacgagctggtggagaagttctacgaccccatgtttaagtatcacctgacagtggcacaggtgaggggaggaatggtgtttgagctggccaatagcatcgtgctgccattcgactgtcgggattacgccgtggtgctgagaaagtacgccgacaaaatctactccatctctatgaagcacccccaggagatgaagacctacagcgtgtccttcgattccctgttttctgccgtgaagaacttcacagagatcgccagcaagttttccgagcggctccaggacttcgataagtccaatcccatcgtgctgaggatgatgaacgaccagctgatgttcctggagcgcgcctttatcgaccctctgggcctgcctgatcggcccttctacagacacgtgatctacgcccctagctcccacaacaagtacgccggcgagtcttttccaggcatctacgacgccctgttcgatatcgagagcaaggtggacccctccaaggcctggggagaggtgaagagacaaatctacgtggcagccttcaccgtgcaggctgcagccgagacactgtccgaggtggcctgataaggtaccatccgaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 17 - DNA, 1017 bp, Artificial Sequence
  Description of Artificial Sequence Synthetic polynucleotide
atggagtctcggaaggacatcaccaaccaggaggagctgtggaagatgaagccacggagaaatctggaggaggacgattacctgcacaaggataccggcgagacatccatgctgaagcggcccgtgctgctgcacctgcaccagaccgcacacgccgacgagtttgattgcccctctgagctgcaacacacacaggagctgttcccacagtggcacctgcccatcaagatcgccgccatcatcgccagcctgacctttctgtatacactgctgagagaagtgatccaccctctggccacctcccaccagcagtacttctataagatccctatcctggtcatcaacaaggtgctgccaatggtgagcatcacactgctggccctggtgtacctgcctggcgtgatcgccgccatcgtgcagctgcacaacggcaccaagtataagaagtttccacactggctggacaagtggatgctgacacgcaagcagttcggcctgctgtctttctttttcgccgtgctgcacgccatctacagcctgtcctatcccatgaggcgcagctacaggtataagctgctgaactgggcctaccagcaggtgcagcagaataaggaggacgcctggatcgagcacgacgtgtggcgcatggaaatctacgtgagcctgggaatcgtgggcctggcaatcctggccctgctggcagtgacctctatcccttctgtgagcgactccctgacctggcgggagtttcactacatccagtctaagctgggcatcgtgagcctgctgctgggcaccatccacgccctgatctttgcctggaacaagtggatcgatatcaagcagttcgtgtggtataccccccccaccttcatgatcgccgtgttcctgcccatcgtggtgctgatctttaagagcatcctgttcctgccttgcctgcggaagaagatcctgaagatcagacacggctgggaggacgtgaccaagatcaataagacagagatttgcagccaattg

SEQ ID NO: 18 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 19 - DNA, 2059 bp, Artificial Sequence
  Description of Artificial Sequence Synthetic polynucleotide
tcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaggatccgccaccatggagtctcggaaggacatcaccaaccaggaggagctgtggaagatgaagccacggagaaatctggaggaggacgattacctgcacaaggataccggcgagacatccatgctgaagcggcccgtgctgctgcacctgcaccagaccgcacacgccgacgagtttgattgcccctctgagctgcaacacacacaggagctgttcccacagtggcacctgcccatcaagatcgccgccatcatcgccagcctgacctttctgtatacactgctgagagaagtgatccaccctctggccacctcccaccagcagtacttctataagatccctatcctggtcatcaacaaggtgctgccaatggtgagcatcacactgctggccctggtgtacctgcctggcgtgatcgccgccatcgtgcagctgcacaacggcaccaagtataagaagtttccacactggctggacaagtggatgctgacacgcaagcagttcggcctgctgtctttctttttcgccgtgctgcacgccatctacagcctgtcctatcccatgaggcgcagctacaggtataagctgctgaactgggcctaccagcaggtgcagcagaataaggaggacgcctggatcgagcacgacgtgtggcgcatggaaatctacgtgagcctgggaatcgtgggcctggcaatcctggccctgctggcagtgacctctatcccttctgtgagcgactccctgacctggcgggagtttcactacatccagtctaagctgggcatcgtgagcctgctgctgggcaccatccacgccctgatctttgcctggaacaagtggatcgatatcaagcagttcgtgtggtataccccccccaccttcatgatcgccgtgttcctgcccatcgtggtgctgatctttaagagcatcctgttcctgccttgcctgcggaagaagatcctgaagatcagacacggctgggaggacgtgaccaagatcaataagacagagatttgcagccaattgtgataactcgagatccgaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 20 - DNA, 883 bp, Artificial Sequence
  Description of Artificial Sequence Synthetic polynucleotide
tcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattgga

SEQ ID NO: 21 - DNA, 135 bp, Artificial Sequence
  Description of Artificial Sequence Synthetic polynucleotide
atccgaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 22 - DNA, 54 bp, Artificial Sequence
  Description of Artificial Sequence Synthetic oligonucleotide
gagctctccctatcagtgatagagatctccctatcagtgatagagatcgtcgac

SEQ ID NO: 23 - DNA, 28 bp, Artificial Sequence
  Description of Artificial Sequence Synthetic oligonucleotide
aacaaacagacaatctggtctgtttgta

SEQ ID NO: 24 - DNA, 829 bp, Human betaherpesvirus 5
tcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattgga

SEQ ID NO: 25 - protein, 232 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 26 - protein, 215 aa, Mus musculus
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTL

SEQ ID NO: 27 - protein, 281 aa, Siniperca chuatsi
MADSAEDAPMARGSLAGSDEALILPAGPTGGSNSRALKVAGLTTLTCLLLASQVFTAYMVFGQKEQIHTLQKNSERMSKQLTRSSQAVAPMKMHMPMNSLPLLMDFTPNEDSKTPLTKLQDTAVVSVEKQLKDLMQDSQLPQFNETFLANLQGLKQQMNESEWKSFESWMRYWLIFQMAQQKPVPPTADPASLIKTKCQMESAPGVSKIGSYKPQCDEQGRYKPMQCWHATGFCWCVDETGAVIEGTTMRGRPDCQRRALAPRRMAFAPSLMQKTISIDDQ

SEQ ID NO: 28 - protein, 16 aa, Siniperca chuatsi
QIHTLQKNSERMSKQL

SEQ ID NO: 29 - protein, 29 aa, Artificial Sequence
  Description of Artificial Sequence Synthetic peptide
MDAMKRGLCCVLLLCGAVFVSPSQEIHAR

SEQ ID NO: 30 - protein, 22 aa, Artificial Sequence
  Description of Artificial Sequence Synthetic peptide
GSGATNFSLLKQAGDVEENPGP

SEQ ID NO: 31 - protein, 21 aa, Artificial Sequence
  Description of Artificial Sequence Synthetic peptide
GSGEGRGSLLTCGDVEENPGP

SEQ ID NO: 32 - protein, 23 aa, Artificial Sequence
  Description of Artificial Sequence Synthetic peptide
GSGQCTNYALLKLAGDVESNPGP

SEQ ID NO: 33 - protein, 9 aa, Artificial Sequence
  Description of Artificial Sequence Synthetic peptide
  MOD_RES (5)..(5): Any amino acid
GDVEXNPGP

SEQ ID NO: 34 - DNA, 6 bp, Artificial Sequence
  Description of Artificial Sequence Synthetic oligonucleotide
tagtaa